(19) United States
(12) Patent Application Publication
(10) Pub. No.: US 2023/0055572 A1
(43) Pub. Date: Feb. 23, 2023

(54) BIOMARKERS FOR DIAGNOSING OVARIAN CANCER

(71) Applicant: Venn Biosciences Corporation, South San Francisco, CA (US)

(72) Inventors: Daniel SERIE, San Mateo, CA (US); Chad Eagle PICKERING, San Mateo, CA (US); Prasanna RAMACHANDRAN, Menlo Park, CA (US); Gege XU, Redwood City, CA (US)

(73) Assignee: Venn Biosciences Corporation, South San Francisco, CA (US)

(21) Appl. No.: 17/747,851

(22) Filed: May 18, 2022

Related U.S. Application Data
(60) Provisional application No. 63/190,141, filed on May 18, 2021.
(60) Provisional application No. 63/307,009, filed on Feb. 4, 2022.

(51) Int. Cl. G16B 25/10; G16B 40/20; G16H 50/20

(52) U.S. Cl. CPC G16B 25/10; G16B 40/20; G16H 50/20

(57) ABSTRACT

Set forth herein are glycopeptide biomarkers useful for diagnosing diseases and conditions, such as ovarian cancer. Also set forth herein are methods of generating glycopeptide biomarkers and methods of analyzing glycopeptides using mass spectroscopy. Also set forth herein are methods of analyzing glycopeptides using machine learning systems.

### CROSS-REFERENCE TO RELATED APPLICATION

The present application claims the benefit of and priority to U.S. Provisional Patent Application No. 63/190,141, filed May 18, 2021, and to U.S. Provisional Patent Application No. 63/307,009, filed Feb. 4, 2022, each which is incorporated herein by reference in its entirety.

### SEQUENCE LISTING PARAGRAPH

The content of the following submission on ASCII text file is incorporated herein by reference in its entirety: a computer readable form (CRF) of the Sequence Listing (file name: 166532002000SEQLIST.TXT, date recorded: May 16, 2022, size: 168,290 bytes).

### FIELD

The instant disclosure is directed to uses and treatments of glycoproteomic biomarkers relating to ovarian cancer. More specifically, the disclosure relates to glycans, peptides, and glycopeptides, as well as to methods of using these biomarkers with mass spectroscopy and in clinical applications to determine the presence, progression or treatment of ovarian cancer in a patient.

### BACKGROUND

Changes in glycosylation have been described in relationship to disease states such as cancer. See, e.g., Dube, D. H.; Bertozzi, C. R. Glycans in Cancer and Inflammation—Potential for Therapeutics and Diagnostics. Nature Rev. Drug Disc. 2005, 4, 477-88, the entire contents of which are herein incorporated by reference in its entirety for all purposes. Conventional clinical assays for diagnosing ovarian cancer, for example, include measuring the amount of the protein CA 125 (cancer antigen 125) in a patient's blood by an enzyme-linked immunosorbent assay (ELIS A).

However, ELISA has limited sensitivity and precision. ELISA, for example, only measures CA 125 at concentrations in the ng/mL range. This narrow measurement range limits the relevance of this assay by failing to measure biomarkers at concentrations substantially above or below this concentration range. Also, the CA 125 ELISA assay is limited with respect to the types of samples which can be assayed. As a consequence of the lack of more precise and sensitive tests, patients who might otherwise be diagnosed with ovarian cancer are not and thereby fail to receive proper follow-up medical attention.

### SUMMARY

Machine learning presents a new technological advancement in the diagnosis and treatment of disease, wherein novel common biomarkers are identified from tissues displaying similar etiologies. This represents a promising advance due, at least in part, to the potential for specifically targeting diseased or damaged cells and identifying cancerous and precancerous tissues using powerful and complex spectrometry-based assays. One promising approach is the identification of glycans, peptides, and glycopeptides, as well as fragments thereof, in some instances using mass spectroscopy to diagnose ovarian cancer.

In one embodiment, set forth herein is a glyopeptide or peptide consisting of an amino acid sequence selected from SEQ ID Nos: 1-38, and combinations thereof.

In another embodiment, set forth herein is a glycopeptide or peptide consisting essentially of an amino acid sequence selected from SEQ ID NOs: 1-38, and combinations thereof.

In another embodiment, set forth herein is a method for detecting one or more MRM transitions, comprising: obtaining a biological sample from a patient; digesting and/or fragmenting a glycopeptide in the sample; and detecting a multiple-reaction-monitoring (MRM) transition selected from the group consisting of transitions 1-38 described herein, particularly with reference to Table 1. In one embodiment, the method includes analyzing a subset of the transitions found in Table 1 to determine if the biological sample is indicative of ovarian cancer. For example, a subset of 10, 15, 16, 18, 20, 25, or 30, or any number of such transitions found in the biological sample may be indicative of ovarian cancer in the patient.

In another embodiment, set forth herein is a method for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of SEQ ID NOs: 1-38, and combinations thereof; and inputting the quantification into a trained model to generate an output probability; determining if the output probability is above or below a threshold for a classification; and identifying a classification for the sample based on whether the output probability is above or below a threshold for a classification.

In yet another embodiment, set forth herein is a method for classifying a biological sample, comprising: obtaining a biological sample from a patient; digesting and/or fragmenting glycopeptides in the sample; detecting a MRM transition selected from the group consisting of transitions 1-38; and quantifying the glycopeptides; inputting the quantification into a trained model to generate a output probability; determining if the output probability is above or below a threshold for a classification; and classifying the biological sample based on whether the output probability is above or below a threshold for a classification.

In another embodiment, set forth herein is a method for treating a patient having ovarian cancer; the method comprising: obtaining a biological sample from the patient; digesting and/or fragmenting one or more glycopeptides in the sample; and detecting and quantifying one or more multiple-reaction-monitoring (MRM) transitions selected from the group consisting of transitions 1-38; inputting the quantification into a trained model to generate an output probability; determining if the output probability is above or below a threshold for a classification; and classifying the patient based on whether the output probability is above or below a threshold for a classification, wherein the classification is selected from the group consisting of: (A) a patient in need of a chemotherapeutic agent; (B) a patient in need of a immunotherapeutic agent; (C) a patient in need of hormone therapy; (D) a patient in need of a targeted therapeutic agent; (E) a patient in need of surgery; (F) a patient in need of neoadjuvant therapy; (G) a patient in need of chemotherapeutic agent, immunotherapeutic agent, hormone therapy, targeted therapeutic agent, neoadjuvant therapy, or a combination thereof, before surgery; (H) a patient in need of chemotherapeutic agent, immunotherapeutic agent, hormone therapy, targeted therapeutic agent, neoadjuvant therapy, or a combination thereof, after surgery; (I) or a combination thereof; administering a therapeutically effective amount of a therapeutic agent to the patient: wherein the therapeutic agent is selected from chemotherapy if classification A or I is determined; wherein the therapeutic agent is selected from immunotherapy if classification B or I is determined; or wherein the therapeutic agent is selected from hormone therapy if classification C or I is determined; or wherein the therapeutic agent is selected from targeted therapy if classification D or I is determined wherein the therapeutic agent is selected from neoadjuvant therapy if classification F or I is determined; wherein the therapeutic agent is selected from chemotherapeutic agent, immunotherapeutic agent, hormone therapy, targeted therapeutic agent, neoadjuvant therapy, or a combination thereof if classification G or I is determined; and wherein the therapeutic agent is selected from chemotherapeutic agent, immunotherapeutic agent, hormone therapy, targeted therapeutic agent, neoadjuvant therapy, or a combination thereof if classification H or I is determined.

In another embodiment, set forth herein is a method for training a machine learning system, comprising: providing a first data set of MRM transition signals indicative of a sample comprising a glycopeptide consisting of, or consisting essentially of, an amino acid sequence selected from the group consisting of SEQ ID NOs: 1-38; providing a second data set of MRM transition signals indicative of a control sample; and comparing the first data set with the second data set using a machine learning system.

In another embodiment, set forth herein is a method for diagnosing a patient having ovarian cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect and quantify one or more MRM transitions selected from transitions 1-38; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having ovarian cancer based on the diagnostic classification. In some examples, the method includes performing mass spectroscopy of the biological sample using MRM-MS with a QQQ.

In another embodiment, set forth herein is a method for diagnosing a patient having ovarian cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect and quantify one or more MRM transitions selected from transitions 1-38; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having ovarian cancer based on the diagnostic classification. In some examples, selecting any of 10, 15, 16, 18, 20, 25, or 30, or any number between 10-30 of the glycopeptides or transitions is sufficient to identify the diagnostic classification; and diagnose the patient as having ovarian cancer based on the diagnostic classification. In another embodiment, set forth herein is a kit comprising a glycopeptide standard, a buffer, and one or more glycopeptides consisting of, or consisting essentially of, an amino acid sequence selected from the group consisting of SEQ ID NOs: 1-38.

In another embodiment, set forth herein is a glycopeptide consisting of, or consisting essentially of, an amino acid sequence selected from the group consisting of SEQ ID NOs: 1-38

In one or more embodiments, a method for diagnosing a subject with respect to an ovarian cancer disease state is described according to various embodiments. In various embodiments, the method may comprise receiving peptide structure data corresponding to a biological sample obtained from the subject. In various embodiments, the method may comprise analyzing the peptide structure data using a supervised machine learning model to generate a disease indicator that indicates whether the biological sample evidences an ovarian cancer disease state based on at least three peptide structures selected from one of a first group of peptide structures identified in Table 1A and a second group of peptide structures identified in Table 2A. In various embodiments, the first group of peptide structures and the second group of peptide structures may be associated with the ovarian cancer disease state. In various embodiments, the first group of peptide structures in Table 1A and the second group of peptide structures in Table 2A may be listed in order of relative significance to the disease indicator. In various embodiments, the method may comprise generating a diagnosis output based on the disease indicator.

In one or more embodiments, a method of training a model to diagnose a subject with respect to an ovarian cancer disease state is described according to various embodiments. In various embodiments, the method comprises receiving quantification data for a panel of peptide structures for a plurality of samples for a plurality of subjects. In various embodiments, the plurality of subjects includes a first portion diagnosed with a negative diagnosis of an ovarian cancer disease state and a second portion diagnosed with a positive diagnosis of the ovarian cancer disease state. In various embodiments, the quantification data comprises a plurality of peptide structure profiles for the plurality of subjects. In various embodiments, the method comprises training a machine learning model using the quantification data to diagnose a biological sample with respect to the ovarian cancer disease state using a first group of peptide structures associated with the ovarian cancer disease state or a second group of peptide structures associated with the ovarian cancer disease state. In various embodiments, the first group of peptide structures may be identified in Table 1A and listed in Table 1A with respect to relative significance to diagnosing the biological sample. In various embodiments, the second group of peptide structures is identified in Table 2A and listed in Table 2A with respect to relative significance to diagnosing the biological sample.

In one or more embodiments, a composition comprising at least one of peptide structures PS-1-PS-10 identified in Table 1A is described according to various embodiments.

In one or more embodiments, a composition comprising at least one of peptide structures PS-11-PS-34 and PS-5 identified in Table 2A is described according to various embodiments.

In one or more embodiments, a composition comprising at least one of peptide structures PS-1-PS-10 and PS-11-PS-34 from Table 1A and Table 2A is described according to various embodiments.

In one or more embodiments, a composition comprising a peptide structure or a product ion is described according to various embodiments. In various embodiments, the peptide structure or the product ion comprises an amino acid sequence having at least 90% sequence identity to any one of SEQ ID NOS: 111-119, corresponding to respective ones of peptide structures PS-1 to PS-10 in Table 1A. In various embodiments, the product ion may be selected as one from a group consisting of product ions corresponding to PS-1 to PS-10 identified in Table 4A including product ions falling within an identified m/z range.

In one or more embodiments, a composition comprising a peptide structure or a product ion is described according to various embodiments. In various embodiments, the peptide structure or the product ion comprises an amino acid sequence having at least 90% sequence identity to any one of SEQ ID NOS: 114, 115, and 131-146 corresponding to respective ones of peptide structures PS-5 and PS-11-PS-34 in Table 2A. In various embodiments, the product ion may be selected as one from a group consisting of product ions corresponding to PS-5 and PS-11-PS-34 identified in Table 2A including product ions falling within an identified m/z range.

In one or more embodiments, a composition comprising a peptide structure or a product ion is described according to various embodiments. In various embodiments, the peptide structure or the product ion comprises an amino acid sequence having at least 90% sequence identity to SEQ ID NO: 115, corresponding to peptide structure PS-5 in Tables 1A, 2A, and 3A. In various embodiments, the product ion may be selected as one from a group consisting of product ions corresponding to PS-5 identified in Table 4A including product ions falling within an identified m/z range.

In one or more embodiments, a composition comprising a glycopeptide structure selected as one from a group consisting of peptide structures PS-1 to PS-10 identified in Table 1A is described according to various embodiments. In various embodiments, the composition comprises an amino acid peptide sequence identified in Table 5A as corresponding to the peptide structure. In various embodiments, the composition comprises a glycan structure identified in Table 7A as corresponding to the peptide structure in which the glycan structure is linked to a residue of the amino acid peptide sequence at a corresponding position identified in Table 1A. In various embodiments, the glycan structure may comprise a glycan composition.

In one or more embodiments, a composition comprising a glycopeptide structure selected as one from a group consisting of peptide structures PS-5 and PS-11-PS-34 identified in Table 2A is described according to various embodiments. In various embodiments, the peptide structure comprises an amino acid peptide sequence identified in Table 5A as corresponding to the peptide structure. In various embodiments, the peptide structure comprises a glycan structure identified in Table 7A as corresponding to the peptide structure in which the glycan structure is linked to a residue of the amino acid peptide sequence at a corresponding position identified in Table 2A. In various embodiments, the glycan structure has a glycan composition.

In one or more embodiments, a composition comprising a peptide structure selected as one from a plurality of peptide structures identified in Table 1A or 2A is described according to various embodiments. In various embodiments, the peptide structure has a monoisotopic mass identified as corresponding to the peptide structure in Table 1A. In various embodiments, the peptide structure comprises the amino acid sequence of SEQ ID NOs: 111-119 identified in Table 1A as corresponding to the peptide structure.

In one or more embodiments, a composition comprising a peptide structure selected as one from a plurality of peptide structures identified in Table 2A is described according to various embodiments. In various embodiments, the peptide structure has a monoisotopic mass identified as corresponding to the peptide structure in Table 2A. In various embodiments, the peptide structure comprises the amino acid sequence of SEQ ID NOS: 114, 115, 131-146 identified in Table 2A as corresponding to the peptide structure.

In one or more embodiments, a kit comprising at least one agent for quantifying at least one peptide structure identified in Table 1A to carry out the method of any one of embodiments 1A-40A is described according to various embodiments.

In one or more embodiments, a kit comprising at least one agent for quantifying at least one peptide structure identified in Table 2A to carry out the method of any one of embodiments 1A-40A is described according to various embodiments.

In one or more embodiments, a kit comprising at least one agent for quantifying at least one peptide structure identified in at least one of Table 1A or Table 2A to carry out the method of any one of embodiments 1A-40A is described according to various embodiments.

In one or more embodiments, a kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 111-119, defined in Table 1A and Table 5A is described according to various embodiments.

In one or more embodiments, a kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 114, 115, and 131-146, defined in Table 2A and Table 5A is described according to various embodiments.

In one or more embodiments, a kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 111-119 and 131-146 defined in Tables 1A, 2A, and 5A is described according to various embodiments.

In one or more embodiments, system comprising one or more data processors is described according to various embodiments. In various embodiments, the system comprises a non-transitory computer readable storage medium containing instructions which, when executed on the one or more data processors, cause the one or more data processors to perform part or all of any one of embodiments 1A-40A.

In one or more embodiments, a computer-program product tangibly embodied in a non-transitory machine-readable storage medium, including instructions configured to cause one or more data processors to perform part or all of any one of embodiments 1A-40A is described according to various embodiments.

In one or more embodiments, a method for diagnosing a subject with respect to an ovarian cancer disease state is described according to various embodiments. In various embodiments, the method comprises receiving peptide structure data corresponding to a biological sample obtained from the subject. In various embodiments, the method comprises analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 3A. In various embodiments, the group of peptide structures in Table 3A is listed in order of relative significance to the disease indicator. In various embodiments, the method comprises generating a diagnosis output based on the disease indicator.

In one or more embodiments, a method of training a model to diagnose a subject with respect to an ovarian cancer disease state having a malignant pelvic tumor is described according to various embodiments. In various embodiments, the method comprises receiving quantification data for a panel of peptide structures for a plurality of samples for a plurality of subjects. In various embodiments, the plurality of subjects includes a first portion diagnosed with a negative diagnosis of an ovarian cancer disease state and a second portion diagnosed with a positive diagnosis of the ovarian cancer disease state. In various embodiments, the quantification data comprises a plurality of peptide structure profiles for the plurality of subjects. In various embodiments, the method comprises training a machine learning model using the quantification data to diagnose a biological sample with respect to the ovarian cancer disease state using a group of peptide structures associated with the ovarian cancer disease state. In various embodiments, the group of peptide structures is identified in Table 3A and listed in Table 3A with respect to relative significance to diagnosing the biological sample.

In one or more embodiments, a composition comprising at least one of peptide structures PS-1, PS-5, PS-11, PS-15, PS-20, PS-25, PS-28, PS-29, PS-30, PS-31, PS-32, and PS-35 to PS-61 identified in Table 3A is described according to various embodiments.

In one aspect, a composition comprising at least one of peptide structures PS-1, PS-5, PS-11, PS-15, PS-20, PS-25, PS-28, PS-29, PS-30, PS-31, PS-32, or PS-35 to PS-61 identified in Table 3A and at least one of peptide structures PS-1-PS-34 in Tables 1A and 2A is described according to various embodiments.

In one or more embodiments, a composition comprising a peptide structure or a product ion is described according to various embodiments. In various embodiments, the peptide structure or the product ion comprises an amino acid sequence having at least 90% sequence identity to any one of SEQ ID NOS: 111, 114, 115, 131, 132, 133, 134, 137, 138, 140, 142, 144, 145, 146, 153-165 corresponding to respective ones of peptide structures PS-1, PS-5, PS-11, PS-15, PS-20, PS-25, PS-28, PS-29, PS-30, PS-31, PS-32, and PS-35 to PS-61 in Table 3A. In various embodiments, the product ion is selected as one from a group consisting of product ions corresponding to PS PS-1, PS-5, PS-11, PS-15, PS-20, PS-25, PS-28, PS-29, PS-30, PS-31, PS-32, and PS-35 to PS-61 identified in Table 3A including product ions falling within an identified m/z range.

In one or more embodiments, a composition comprising a glycopeptide structure selected as one from a group consisting of peptide structures PS-1, PS-5, PS-11, PS-15, PS-20, PS-25, PS-28, PS-29, PS-30, PS-31, PS-32, and PS-35 to PS-61 identified in Table 3A is described according to various embodiments. In various embodiments, the peptide structure comprises an amino acid peptide sequence identified in Table 5A as corresponding to the peptide structure. In various embodiments, a glycan structure identified in Table 7A as corresponding to the peptide structure in which the glycan structure is linked to a residue of the amino acid peptide sequence at a corresponding position identified in Table 3A. In various embodiments, the glycan structure has a glycan composition. In various embodiments, a composition comprising a peptide structure selected as one from a plurality of peptide structures identified in Table 3A. In various embodiments, the peptide structure has a monoisotopic mass identified as corresponding to the peptide structure in Table 3A. In various embodiments, the peptide structure comprises the amino acid sequence of SEQ ID NOS: 111, 114, 115, 131, 132, 133, 134, 137, 138, 140, 142, 144, 145, 146, 153-165 identified in Table 3A as corresponding to the peptide structure.

In one or more embodiments, a kit comprising at least one agent for quantifying at least one peptide structure identified in Table 3A to carry out the method of any one of embodiments 76A-110A is described according to various embodiments.

In one or more embodiments, a kit comprising at least one of a glycopeptide standard, a buffer, or a set of peptide sequences to carry out the method of any one of embodiments 76A-110A, a peptide sequence of the set of peptide sequences identified by a corresponding one of SEQ ID NOS: 111, 114, 115, 131, 132, 133, 134, 137, 138, 140, 142, 144, 145, 146, 153-165 identified in Table 3A is described according to various embodiments.

In one or more embodiments, a system comprising one or more data processors is described according to various embodiments. In various embodiments, the system comprises a non-transitory computer readable storage medium containing instructions which, when executed on the one or more data processors, cause the one or more data processors to perform part or all of any one of embodiments 76A-110A.

In one or more embodiments, a computer-program product tangibly embodied in a non-transitory machine-readable storage medium, including instructions configured to cause one or more data processors to perform part or all of any one of embodiments 76A-110A is described according to various embodiments.

In one or more embodiments, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one or more embodiments, a computer-program product tangibly embodied in a non-transitory machine-readable storage medium, including instructions configured to cause one or more data processors to perform part or all of any one or more of the methods described herein.

### BRIEF DESCRIPTION OF THE DRAWINGS

FIG. 1 illustrates glycan chemical structures, using the Symbol Nomenclature for Glycans (SNFG) system. Each glycan structure is associated with a glycan reference code number from 3200-3600.

FIG. 2 illustrates glycan chemical structures, using the Symbol Nomenclature for Glycans (SNFG) system. Each glycan structure is associated with a glycan reference code number from 3610-4301.

FIG. 3 illustrates glycan chemical structures, using the Symbol Nomenclature for Glycans (SNFG) system. Each glycan structure is associated with a glycan reference code number from 4310-4531.

FIG. 4 illustrates glycan chemical structures, using the Symbol Nomenclature for Glycans (SNFG) system. Each glycan structure is associated with a glycan reference code number from 4541-4710.

FIG. 5 illustrates glycan chemical structures, using the Symbol Nomenclature for Glycans (SNFG) system. Each glycan structure is associated with a glycan reference code number from 4711-5400.

FIG. 6 illustrates glycan chemical structures, using the Symbol Nomenclature for Glycans (SNFG) system. Each glycan structure is associated with a glycan reference code number from 5401-5420.

FIG. 7 illustrates glycan chemical structures, using the Symbol Nomenclature for Glycans (SNFG) system. Each glycan structure is associated with a glycan reference code number from 5421-5731.

FIG. 8 illustrates glycan chemical structures, using the Symbol Nomenclature for Glycans (SNFG) system. Each glycan structure is associated with a glycan reference code number from 6200-6402.

FIG. 9 illustrates glycan chemical structures, using the Symbol Nomenclature for Glycans (SNFG) system. Each glycan structure is associated with a glycan reference code number 6410-6511.

FIG. 10 illustrates glycan chemical structures, using the Symbol Nomenclature for Glycans (SNFG) system. Each glycan structure is associated with a glycan reference code number from 6512-6632.

FIG. 11 illustrates glycan chemical structures, using the Symbol Nomenclature for Glycans (SNFG) system. Each glycan structure is associated with a glycan reference code number from 6641-7410.

FIG. 12 illustrates glycan chemical structures, using the Symbol Nomenclature for Glycans (SNFG) system. Each glycan structure is associated with a glycan reference code number from 7411-7601.

FIG. 13 illustrates glycan chemical structures, using the Symbol Nomenclature for Glycans (SNFG) system. Each glycan structure is associated with a glycan reference code number from 7602-7741.

FIG. 14 illustrates glycan chemical structures, using the Symbol Nomenclature for Glycans (SNFG) system. Each glycan structure is associated with a glycan reference code number from 8200-11200.

FIG. 15 shows a workflow for detecting transitions by mass spectroscopy.

FIG. 16 shows a one pot workflow for detecting transitions by mass spectroscopy.

FIG. 17 is a plot of intensity by retention time (RT) obtained by liquid chromatography/mass spectrometry (LC/MS) detection of a biomarker analyte. The top plot shows predicted probabilities from the PB-Net system process, and a final (RT) start and stop prediction for the integrated peak.

FIG. 18 shows LC retention time analysis.

FIG. 19 is a schematic diagram of an exemplary workflow 100 for the detection of peptide structures associated with a disease state for use in diagnosis and/or treatment in accordance with one or more embodiments.

FIG. 20A is a schematic diagram of a preparation workflow in accordance with one or more embodiments.

FIG. 20B is a schematic diagram of data acquisition in accordance with one or more embodiments.

FIG. 21 is a block diagram of an analysis system in accordance with one or more embodiments.

FIG. 22 is a block diagram of a computer system in accordance with various embodiments.

FIG. 23 is a flowchart of a process for diagnosing a subject with respect to an ovarian cancer disease state in accordance with one or more embodiments.

FIG. 24 is a flowchart of a process for training a model to diagnose a subject with respect to ovarian cancer disease state in accordance with one or more embodiments.

FIG. 25 is a flowchart of a process for training a model to diagnose a subject with respect to an ovarian cancer disease state in accordance with one or more embodiments.

FIG. 26 is a table describing the distribution of the samples acquired in this exemplary retrospective analysis in accordance with one or more embodiments.

FIG. 27 is a plot diagram illustrating the results of a principal component analysis performed to assess the segregation between healthy, benign pelvic tumor, and EOC samples across first and second principal components in accordance with one or more embodiments.

FIG. 28 is a plot diagram illustrating the results of a principal component analysis performed to assess segregation between healthy, benign pelvic tumor, early EOC, late EOC, and missing (undocumented) samples).

FIG. 29 is an illustration of a receiver operating characteristic (ROC) diagram corresponding to the multivariable model built to predict malignancy v. benign status of pelvic tumors in accordance with one or more embodiments.

FIG. 30 is an illustration of a diagram showing the probability distributions for the various groups using the multivariable model for predicting malignancy v. benign status of pelvic tumors in accordance with one or more embodiments.

FIG. 31 is an illustration of a receiver operating characteristic (ROC) diagram corresponding to the multivariable model built to predict malignancy v. benign status of pelvic tumors in accordance with one or more embodiments.

FIG. 32 is an illustration of a diagram showing the probability distributions for the various groups using the multivariable model for predicting malignancy v. benign status of pelvic tumors in accordance with one or more embodiments.

FIG. 33 is a visualization of top two principal components in PCA of all 351 subjects included in the analysis (subjects are colored by phenotype, with malignant EOC subjects stratified by stage group on the right).

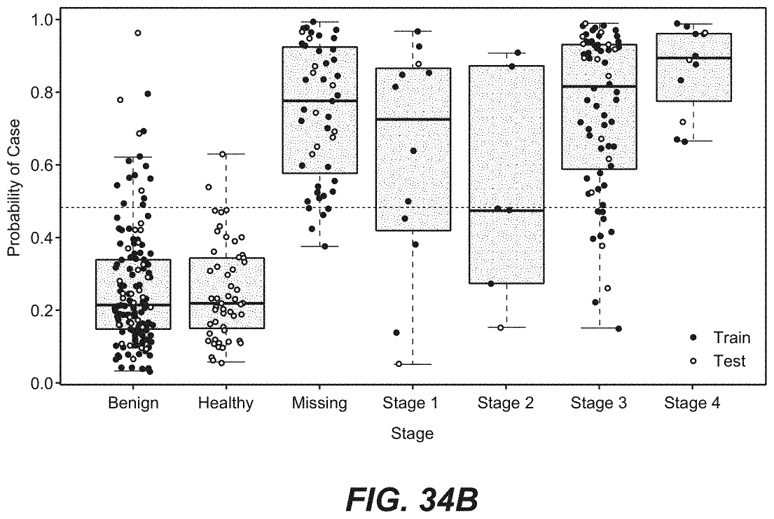

FIG. 34 is a ROC analysis of glycoforms distinguishing EOC from benign masses (34a). The resultant distribution of predicted probabilities indicates a well-trained model (34b), and application to blinded healthy patients and increasing severity with disease progression indicate a link to the biology of disease.

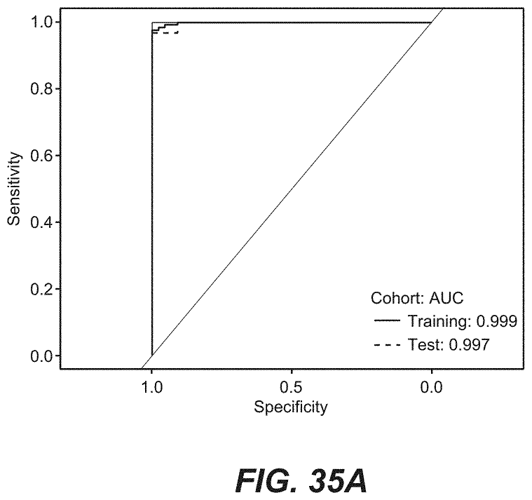

FIG. 35 is a ROC analysis that strongly distinguishes ovarian cancer from a healthy state (35a). The resultant distribution of predicted probabilities indicates a well-trained model (35b). Application to blinded benign mass patients resulted in most above the cutoff, indicating the signature is primarily predictive of the presence of a mass and less of its nature.

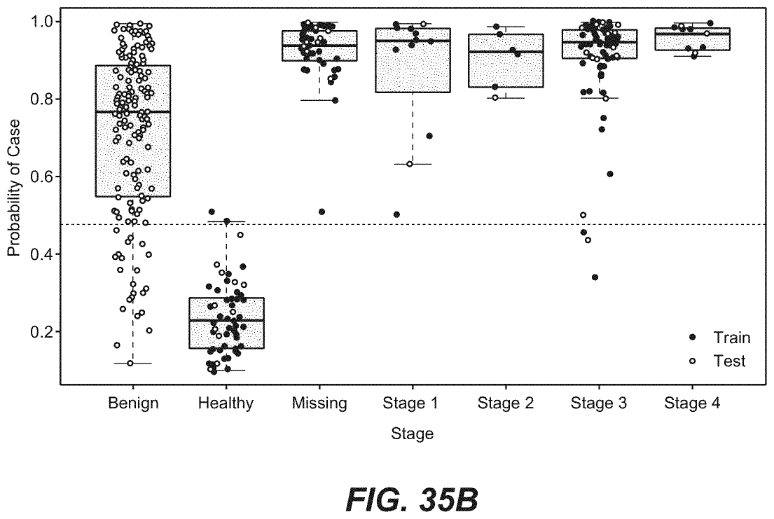

FIG. 36 is a Venn diagram. Among the selected top-ranked differentially expressed glycopeptide features, the Venn diagram shows the overlaps between and among study contrasts. 50, 40, and 36 features were found to differ among benign disease vs. healthy, early disease vs. healthy, and late-stage disease vs. healthy phenotypes, respectively; 22 features were found in both early-stage disease vs. healthy and the late-stage vs. healthy comparisons; 12 features were found in both benign disease vs. healthy and late-stage disease vs. healthy comparisons; 8 features were found in both benign disease vs. healthy and early disease vs. healthy comparisons; and 39 features were found in common across all comparisons.

The patent or application file contains at least one drawing executed in color. Copies of this patent or patent application publication with color drawing(s) will be provided by the Office upon request and payment of the necessary fee.

### DETAILED DESCRIPTION

The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the inventions herein are not intended to be limited to the embodiments presented, but are to be accorded their widest scope consistent with the principles and novel features disclosed herein.

All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

### I. GENERAL

The instant disclosure provides methods and compositions for the profiling, detecting, and/or quantifying of glycans in a biological sample. In some examples, glycan and glycopeptide panels are described for diagnosing and screening patients having ovarian cancer. In some examples, glycan and glycopeptide panels are described for diagnosing and screening patients having cancer, an autoimmune disease, or fibrosis.

Certain techniques for analyzing biological samples using mass spectroscopy are known. See, for example, International PCT Patent Application Publication No. WO2019079639A1, filed Oct. 18, 2018 as International Patent Application No. PCT/US2018/56574, and titled IDENTIFICATION AND USE OF BIOLOGICAL PARAMETERS FOR DIAGNOSIS AND TREATMENT MONITORING, the entire contents of which are herein incorporated by reference in its entirety for all purposes. See, also, US Patent Application Publication No. US20190101544A1, filed Aug. 31, 2018 as U.S. patent application Ser. No. 16/120,016, and titled IDENTIFICATION AND USE OF GLYCOPEPTIDES AS BIOMARKERS FOR DIAGNOSIS AND TREATMENT MONITORING, the entire contents of which are herein incorporated by reference in its entirety for all purposes.

### II. BIOMARKERS

Set forth herein are biomarkers. These biomarkers are useful for a variety of applications, including, but not limited to, diagnosing diseases and conditions. For example, certain biomarkers set forth herein, or combinations thereof, are useful for diagnosing ovarian cancer. In some other examples, certain biomarkers set forth herein, or combinations thereof, are useful for diagnosing and screening patients having cancer, an autoimmune disease, or fibrosis. In some examples, the biomarkers set forth herein, or combinations thereof, are useful for classifying a patient so that the patient receives the appropriate medical treatment. In some other examples, the biomarkers set forth herein, or combinations thereof, are useful for treating or ameliorating a disease or condition in patient by, for example, identifying a therapeutic agent with which to treat a patient. In some other examples, the biomarkers set forth herein, or combinations thereof, are useful for determining a prognosis of treatment for a patient or a likelihood of success or survivability for a treatment regimen.

In some examples, a sample from a patient is analyzed by MS and the results are used to determine the presence, absolute amount, and/or relative amount of a glycopeptide consisting of an amino acid sequence selected from SEQ ID NOs: 1-38 in the sample. In some examples, a sample from a patient is analyzed by MS and the results are used to determine the presence, absolute amount, and/or relative amount of a glycopeptide consisting essentially of an amino acid sequence selected from SEQ ID NOs: 1-38 in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 1-38 in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

Set forth herein are biomarkers selected from glycans, peptides, glycopeptides, fragments thereof, and combinations thereof. In some examples, the glycopeptide consists of an amino acid sequence selected from SEQ ID NOs: 1-38. In some examples, the glycopeptide consists essentially of an amino acid sequence selected from SEQ ID NOs: 1-38.

a. O-Glycosylation

In some examples, the glycopeptides set forth herein include O-glycosylated peptides. These peptides include glycopeptides in which a glycan is bonded to the peptide through an oxygen atom of an amino acid. Typically, the amino acid to which the glycan is bonded is threonine (T) or serine (S). In some examples, the amino acid to which the glycan is bonded is threonine (T). In some examples, the amino acid to which the glycan is bonded is serine (S).

In certain examples, the O-glycosylated peptides include those peptides from the group selected from Apolipoprotein C-III (APOC3), Alpha-2-HS-glycoprotein (FETUA), and combinations thereof. In certain examples, the O-glycosylated peptide, set forth herein, is an Apolipoprotein C-III (APOC3) peptide. In certain examples, the O-glycosylated peptide, set forth herein, is an Alpha-2-HS-glycoprotein (FETUA).

b. N-Glycosylation

In some examples, the glycopeptides set forth herein include N-glycosylated peptides. These peptides include glycopeptides in which a glycan is bonded to the peptide through a nitrogen atom of an amino acid. Typically, the amino acid to which the glycan is bonded is asparagine (N) or arginine (R). In some examples, the amino acid to which the glycan is bonded is asparagine (N). In some examples, the amino acid to which the glycan is bonded is arginine (R).

In certain examples, the N-glycosylated peptides include members selected from the group consisting of Alpha-1-antitrypsin (A1AT), Alpha-1B-glycoprotein (A1BG), Leucine-richAlpha-2-glycoprotein (A2GL), Alpha-2-macroglobulin (A2MG), Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-I (APOA1), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), ComplementFactorH (CFAH), ComplementFactorI (CFAI), Clusterin (CLUS), ComplementC3 (CO3), ComplementC4-A&B (CO4A&CO4B), ComplementcomponentC6 (CO6), ComplementComponentC8AChain (CO8A), Coagulation factor XII (FA12), Haptoglobin (HPT), Histidine-rich Glycoprotein (HRG), Immunoglobulin heavy constant alpha 1&2 (IgA12), Immunoglobulin heavy constant alpha 2 (IgA2), Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1), Kininogen-1 (KNG1), Serum paraoxonase/arylesterase 1 (PON1), Selenoprotein P (SEPP1), Prothrombin (THRB), Serotransferrin (TRFE), Transthyretin (TTR), Protein unc-13HomologA (UN13A), Vitronectin (VTNC), Zinc-alpha-2-glycoprotein (ZA2G), Insulin-like growth factor-II (IGF2), Apolipoprotein C-I (APOC1), and combinations thereof.

c. Peptides and Glycopeptides

In some examples, set forth herein is a glycopeptide consisting of an amino acid sequence selected from the group consisting of SEQ ID NOs: 1-38, and combinations thereof.

In some examples, set forth herein is a glycopeptide consisting essentially of an amino acid sequence selected from the group consisting of SEQ ID NOs: 1-38, and combinations thereof.

In certain examples, the glycopeptide consists essentially of an amino acid sequence selected from SEQ ID NO:1. In some examples, the glycopeptide comprises glycan 6513 at residue 107. In some examples, the glycopeptide is A1AT-GP001_107_6513, or alternatively, A1AT_107_6513. Herein A1 AT refers to Alpha-1- antitrypsin.

In certain examples, the glycopeptide consists essentially of an amino acid sequence selected from SEQ ID NO:2. In some examples, the glycopeptide comprises glycan 5411 at residue 1424. In some examples, the glycopeptide is A2MG-GP004_1424_5411 or alternatively, A2MG_1424_5411. Herein A2MG refers to Alpha-2-macroglobulin.

In certain examples, the glycopeptide consists essentially of an amino acid sequence selected from SEQ ID NO:3. In some examples, the glycopeptide comprises glycan 5411 at residue 55. In some examples, the glycopeptide is A2MG-GP004_1424_5411, or alternatively, A2MG_55_5411.

In certain examples, the glycopeptide consists essentially of an amino acid sequence selected from SEQ ID NO:4. In some examples, the glycopeptide comprises glycan 7614 at residue 106. In some examples, the glycopeptide is AACT-GP005_106_7614, or alternatively, AACT_106_7614. Herein AACT refers to Alpha-1-antichymotrypsin.

In certain examples, the glycopeptide consists essentially of an amino acid sequence selected from SEQ ID NO:5. In some examples, the glycopeptide comprises glycan 6513 at residue 271. In some examples, the glycopeptide is AACT-GP005_271_6513, or alternatively, AACT_271_6513.

In certain examples, the glycopeptide consists essentially of an amino acid sequence selected from SEQ ID NO:6. In some examples, the glycopeptide comprises glycan 7603 at residue 103. In some examples, the glycopeptide is AGP1-GP007_103_7603, or alternatively, AGP1_103_7603. Herein, AGP1 refers to Alpha-1-acid glycoprotein 1.

In certain examples, the glycopeptide consists essentially of an amino acid sequence selected from SEQ ID NO:7. In some examples, the glycopeptide comprises glycan 8704 at residue 103. In some examples, the glycopeptide is AGP1-GP007_103_8704, or alternatively, AGP1_103_8704. Herein, AGP1 refers to Alpha-1-acid glycoprotein 1.

In certain examples, the glycopeptide consists essentially of an amino acid sequence selected from SEQ ID NO:8. In some examples, the glycopeptide comprises glycan 9804 at residue 103. In some examples, the glycopeptide is AGP1-GP007_103_9804, or alternatively, AGP1_103_9804. Herein, AGP1 refers to Alpha-1-acid glycoprotein 1.

In certain examples, the glycopeptide consists essentially of an amino acid sequence selected from SEQ ID NO:9. In some examples, the glycopeptide comprises glycan 7614 at residue 93. In some examples, the glycopeptide is AGP1-GP007_93_7614, or alternatively, AGP1_93_7614. Herein, AGP1 refers to Alpha-1-acid glycoprotein 1.

In certain examples, the glycopeptide consists essentially of an amino acid sequence selected from SEQ ID NO:10. In some examples, the glycopeptide comprises glycan 5411 at residue 98. In some examples, the glycopeptide is APOD-GP014_98_5411, or alternatively, APOD_98_5411. Herein, APOD refers to Apolipoprotein D.

In certain examples, the glycopeptide consists essentially of an amino acid sequence selected from SEQ ID NO:11. In some examples, the glycopeptide comprises glycan 9800 at residue 98. In some examples, the glycopeptide is APOD-GP014_98_9800, or alternatively, APOD_98_9800. Herein, APOD refers to Apolipoprotein D.

In certain examples, the glycopeptide consists essentially of an amino acid sequence selected from SEQ ID NO:12. In some examples, the glycopeptide comprises glycan 5402 at residue 221. In some examples, the glycopeptide is C4BPA-GP076_221_5402, or alternatively, C4BPA_221_5402. Herein, C4BPA refers to C4b-binding protein alpha chain.

In certain examples, the glycopeptide consists essentially of an amino acid sequence selected from SEQ ID NO:13. In some examples, the glycopeptide comprises glycan 6502 at residue 138. In some examples, the glycopeptide is CERU-GP023_138_6521, or alternatively, CERU_138_6502. Herein, CERU refers to Ceruloplasmin.

In certain examples, the glycopeptide consists essentially of an amino acid sequence selected from SEQ ID NO:14. In some examples, the glycopeptide comprises glycan 5200 at residue 621. In some examples, the glycopeptide is CO2_621_5200. Herein, CO2 refers to Complement C2.

In certain examples, the glycopeptide consists essentially of an amino acid sequence selected from SEQ ID NO:15. In some examples, the glycopeptide comprises glycan 5401 at residue 176 In some examples, the glycopeptide is FETUA-GP036_176_5401. Herein, FETUA refers to Alpha-2-HS-glycoprotein.

In certain examples, the glycopeptide consists essentially of an amino acid sequence selected from SEQ ID NO:16. In some examples, the glycopeptide comprises glycan 6513 at residue 176 In some examples, the glycopeptide is FETUA-GP036_176_6513. Herein, FETUA refers to Alpha-2-HS-glycoprotein.

In certain examples, the glycopeptide consists essentially of an amino acid sequence selected from SEQ ID NO:17. In some examples, the glycopeptide comprises glycan 1102 at residue 346 In some examples, the glycopeptide is FETUA-GP036_346_1102. Herein, FETUA refers to Alpha-2-HS-glycoprotein.

In certain examples, the glycopeptide consists essentially of an amino acid sequence selected from SEQ ID NO:18. In some examples, the glycopeptide comprises either glycans 5402 or 5421, or both, wherein the glycan(s) are bonded to residue 453. In some examples, the glycopeptide is HEMO-GP042_453_5402/5421. Herein, HEMO refers to Hemopexin.

In certain examples, the glycopeptide consists essentially of an amino acid sequence selected from SEQ ID NO:19. In some examples, the glycopeptide comprises glycan 3410 at residue 297. In some examples, the glycopeptide is IgG1-GP048_297_3410. Herein, IgG refers to Immunoglobulin Heavy Constant Gamma 1.

In certain examples, the glycopeptide consists essentially of an amino acid sequence selected from SEQ ID NO:20. In some examples, the glycopeptide comprises glycan 5510 at residue 297. In some examples, the glycopeptide is IgG1-GP048_297_5510. Herein, IgG refers to Immunoglobulin Heavy Constant Gamma 1.

In certain examples, the glycopeptide consists essentially of an amino acid sequence selected from SEQ ID NO:21. In some examples, the glycopeptide comprises glycan 4510 at residue 297. In some examples, the glycopeptide is IgG2-GP048_297_4510. Herein, IgG refers to Immunoglobulin Heavy Constant Gamma 2.

In certain examples, the glycopeptide consists essentially of an amino acid sequence selected from SEQ ID NO:22. In some examples, the glycopeptide comprises glycan 5400 at residue 297. In some examples, the glycopeptide is IgG2-GP048_297_5400. Herein, IgG refers to Immunoglobulin Heavy Constant Gamma 2.

In certain examples, the glycopeptide consists essentially of an amino acid sequence selected from SEQ ID NO:23. In some examples, the glycopeptide comprises glycan 5510 at residue 297. In some examples, the glycopeptide is IgG2-GP048_297_5510. Herein, IgG refers to Immunoglobulin Heavy Constant Gamma 2.

In certain examples, the glycopeptide consists essentially of an amino acid sequence selected from SEQ ID NO:24. In some examples, the glycopeptide comprises glycan 6501 at residue 324. In some examples, the glycopeptide is PON1-GP060_324_6501. Herein, PON refers to Serum paraoxonase/arylesterase 1.

In certain examples, the glycopeptide consists essentially of an amino acid sequence selected from SEQ ID NO:25. In some examples, the glycopeptide comprises glycan 6501 at residue 324. In some examples, the glycopeptide is PON1-GP060_324_6501. Herein, PON refers to Serum paraoxonase/arylesterase 1.

In certain examples, the peptide comprises an amino acid sequence selected from SEQ ID NO:26. In some examples, the glycopeptide is QuantPep-A2GL-GP003. Herein A2GL refers to Leucine-richAlpha-2-glycoprotein.

In certain examples, the peptide comprises an amino acid sequence selected from SEQ ID NO:27. In some examples, the glycopeptide is QuantPep-AFAM-GP006. Herein, AFAM refers to Afamin.

In certain examples, the peptide comprises an amino acid sequence selected from SEQ ID NO:33. In some examples, the glycopeptide is QuantPep-CAN3-GP022. Herein, CAN3 refers to Calpain-3.

In certain examples, the glycopeptide consists essentially of an amino acid sequence selected from SEQ ID NO:28. In some examples, the glycopeptide is QuantPep-TTR-GP065. Herein TTR refers to Transthyretin.

In certain examples, the glycopeptide consists essentially of an amino acid sequence selected from SEQ ID NO:29. In some examples, the glycopeptide is QuantPep-UN13A-GP066. Herein UN13A refers to Protein unc-13HomologA.

In certain examples, the glycopeptide consists essentially of an amino acid sequence selected from SEQ ID NO:30. In some examples, the glycopeptide comprises glycan 6501 at residue 432. In some examples, the glycopeptide is TRFE-GP064_432_6501. Herein TRFE refers to Serotransferrin.

In certain examples, the glycopeptide consists essentially of an amino acid sequence selected from SEQ ID NO:31. In some examples, the glycopeptide comprises glycan 6502 at residue 432. In some examples, the glycopeptide is TRFE-GP064_432_6502. Herein TRFE refers to Serotransferrin.

In certain examples, the glycopeptide consists essentially of an amino acid sequence selected from SEQ ID NO:32. In some examples, the glycopeptide comprises glycan 6503 at residue 432. In some examples, the glycopeptide is TRFE-GP064_432_6503. Herein TRFE refers to Serotransferrin.

In certain examples, the glycopeptide consists essentially of an amino acid sequence selected from SEQ ID NO:33. In some examples, the glycopeptide comprises glycan 5400 at residue 630. In some examples, the glycopeptide is TRFE-GP064_630_5400. Herein TRFE refers to Serotransferrin.

In certain examples, the glycopeptide consists essentially of an amino acid sequence selected from SEQ ID NO:34. In some examples, the glycopeptide comprises glycan 5411 at residue 630. In some examples, the glycopeptide is TRFE-GP064_630_5411. Herein TRFE refers to Serotransferrin.

In certain examples, the glycopeptide consists essentially of an amino acid sequence selected from SEQ ID NO:35. In some examples, the glycopeptide comprises glycan 6502 at residue 630. In some examples, the glycopeptide is TRFE-GP064_630_6502. Herein TRFE refers to Serotransferrin.

In certain examples, the glycopeptide consists essentially of an amino acid sequence selected from SEQ ID NO:36. In some examples, the glycopeptide comprises glycan 6513 at residue 630. In some examples, the glycopeptide is TRFE-GP064_630_6513. Herein TRFE refers to Serotransferrin.

In certain examples, the glycopeptide consists essentially of an amino acid sequence selected from SEQ ID NO:37. In some examples, the glycopeptide comprises glycan 5401 at residue 169. In some examples, the glycopeptide is VTNC-GP067_169_5401. Herein TRFE refers to Serotransferrin.

In certain examples, the glycopeptide consists essentially of an amino acid sequence selected from SEQ ID NO:38. In some examples, the glycopeptide comprises glycan 5402 at residue 128. In some examples, the glycopeptide is ZA2G-GP068_128_5402. Herein TRFE refers to Serotransferrin.

In some examples, including any of the foregoing, the glycopeptide is a combination of amino acid sequences selected from SEQ ID NOs:1-38.

### III. METHODS OF USING BIOMARKERS

A. Methods for Detecting Glycopeptides

In some embodiments, set forth herein is a method for detecting one or more a multiple-reaction-monitoring (MRM) transition, comprising: obtaining a biological sample from a patient, wherein the biological sample comprises one or more glycopeptides; digesting and/or fragmenting a glycopeptide in the sample; and detecting a multiple-reaction-monitoring (MRM) transition selected from the group consisting of transitions 1-38. These transitions may include, in various examples, any one or more of the transitions in Tables (1-5). These transitions may be indicative of glycopeptides.

In some examples, set forth herein is a method of detecting one or more glycopeptides, wherein each glycopeptide is individually in each instance selected from a glycopeptide consisting of an amino acid sequence selected from the group consisting of SEQ ID NOs: 1-38, and combinations thereof.

In some examples, set forth herein is a method of detecting one or more glycopeptides, wherein each glycopeptide is individually in each instance selected from a glycopeptide consisting essentially of an amino acid sequence selected from the group consisting of SEQ ID NOs: 1-38, and combinations thereof.

In some examples, set forth herein is a method of detecting one or more glycopeptides. In some examples, set forth herein is a method of detecting one or more glycopeptide fragments. In certain examples, the method includes detecting the glycopeptide group to which the glycopeptide, or fragment thereof, belongs. In some of these examples, the glycopeptide group is selected from Alpha-1-antitrypsin (A1AT), Alpha-1B-glycoprotein (A1BG), Leucine-richAlpha-2-glycoprotein (A2GL), Alpha-2-macroglobulin (A2MG), Alpha-1-antichymotrypsin (AACT), Afamin (AFAM), Alpha-1-acid glycoprotein 1 & 2 (AGP12), Alpha-1-acid glycoprotein 1 (AGP1), Alpha-1-acid glycoprotein 2 (AGP2), Apolipoprotein A-I (APOA1), Apolipoprotein C-III (APOC3), Apolipoprotein B-100 (APOB), Apolipoprotein D (APOD), Beta-2-glycoprotein-1 (APOH), Apolipoprotein M (APOM), Attractin (ATRN), Calpain-3 (CAN3), Ceruloplasmin (CERU), ComplementFactorH (CFAH), ComplementFactorI (CFAI), Clusterin (CLUS), ComplementC3 (CO3), ComplementC4-A&B (CO4A&CO4B), ComplementcomponentC6 (CO6), ComplementComponentC8AChain (CO8A), Coagulation factor XII (FA12), Alpha-2-HS-glycoprotein (FETUA), Haptoglobin (HPT), Histidine-rich Glycoprotein (HRG), Immunoglobulin heavy constant alpha 1&2 (IgA12), Immunoglobulin heavy constant alpha 2 (IgA2), Immunoglobulin heavy constant gamma 2 (IgG2), Immunoglobulin heavy constant mu (IgM), Inter-alpha-trypsin inhibitor heavy chain H1 (ITIH1), Plasma Kallikrein (KLKB1), Kininogen-1 (KNG1), Serum paraoxonase/arylesterase 1 (PON1), Selenoprotein P (SEPP1), Prothrombin (THRB), Serotransferrin (TRFE), Transthyretin (TTR), Protein unc-13HomologA (UN13A), Vitronectin (VTNC), Zinc-alpha-2-glycoprotein (ZA2G), Insulin-like growth factor-II (IGF2), Apolipoprotein C-I (APOC1), and combinations thereof.

In some examples, including any of the foregoing, the method includes detecting a glycopeptide, a glycan on the glycopeptide and the glycosylation site residue where the glycan bonds to the glycopeptide. In certain examples, the method includes detecting a glycan residue. In some examples, the method includes detecting a glycosylation site on a glycopeptide. In some examples, this process is accomplished with mass spectroscopy used in tandem with liquid chromatography.

In some examples, including any of the foregoing, the method includes obtaining a biological sample from a patient. In some examples, the biological sample is synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humour, transudate, or combinations of the foregoing. In certain examples, the biological sample is selected from the group consisting of blood, plasma, saliva, mucus, urine, stool, tissue, sweat, tears, hair, or a combination thereof. In some of these examples, the biological sample is a blood sample. In some of these examples, the biological sample is a plasma sample. In some of these examples, the biological sample is a saliva sample. In some of these examples, the biological sample is a mucus sample. In some of these examples, the biological sample is a urine sample. In some of these examples, the biological sample is a stool sample. In some of these examples, the biological sample is a sweat sample. In some of these examples, the biological sample is a tear sample. In some of these examples, the biological sample is a hair sample.

In some examples, including any of the foregoing, the method also includes digesting and/or fragmenting a glycopeptide in the sample. In certain examples, the method includes digesting a glycopeptide in the sample. In certain examples, the method includes fragmenting a glycopeptide in the sample. In some examples, the digested or fragmented glycopeptide is analyzed using mass spectroscopy. In some examples, the glycopeptide is digested or fragmented in the solution phase using digestive enzymes. In some examples, the glycopeptide is digested or fragmented in the gaseous phase inside a mass spectrometer, or the instrumentation associated with a mass spectrometer. In some examples, the mass spectroscopy results are analyzed using machine learning systems. In some examples, the mass spectroscopy results are the quantification of the glycopeptides, glycans, peptides, and fragments thereof. In some examples, this quantification is used as an input in a trained model to generate an output probability. The output probability is a probability of being within a given category or classification, e.g., the classification of having ovarian cancer or the classification of not having ovarian cancer. In some other examples, the output probability is a probability of being within a given category or classification, e.g., the classification of having cancer or the classification of not having cancer. In some examples, the output probability can be quantified by selecting a minimum of 10, 15, 16, 18, 20, 25, or 30, of the glycopeptide sequences shown in SEQ ID Nos. 1-38. In some other examples, the output probability is a probability of being within a given category or classification, e.g., the classification of having an autoimmune disease or the classification of not having an autoimmune disease. In some other examples, the output probability is a probability of being within a given category or classification, e.g., the classification of having fibrosis or the classification of not having an fibrosis.

In some examples, including any of the foregoing, the method includes introducing the sample, or a portion thereof, into a mass spectrometer.

In some examples, including any of the foregoing, the method includes fragmenting a glycopeptide in the sample after introducing the sample, or a portion thereof, into the mass spectrometer.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay is used in combination with mass spectroscopy. In some examples, the immunoassay measures CA-125 and HE4.

In some examples, including any of the foregoing, the method includes digesting a glycopeptide in the sample occurs before introducing the sample, or a portion thereof, into the mass spectrometer.

In some examples, including any of the foregoing, the method includes fragmenting a glycopeptide in the sample to provide a glycopeptide ion, a peptide ion, a glycan ion, a glycan adduct ion, or a glycan fragment ion.

In some examples, including any of the foregoing, the method includes digesting and/or fragmenting a glycopeptide in the sample to provide a glycopeptide consisting of an amino acid sequence selected from the group consisting of SEQ ID NOs:1-38, and combinations thereof.

In some examples, including any of the foregoing, the method includes digesting and/or fragmenting a glycopeptide in the sample to provide a glycopeptide consisting essentially of an amino acid sequence selected from the group consisting of SEQ ID NOs:1-38, and combinations thereof.

In some examples, including any of the foregoing, the method includes digesting a glycopeptide in the sample to provide a glycopeptide consisting of an amino acid sequence selected from the group consisting of SEQ ID NOs:1-38, and combinations thereof.

In some examples, including any of the foregoing, the method includes digesting a glycopeptide in the sample to provide a glycopeptide consisting essentially of an amino acid sequence selected from the group consisting of SEQ ID NOs:1-38, and combinations thereof.

In some examples, including any of the foregoing, the method includes fragmenting a glycopeptide in the sample to provide a glycopeptide consisting of an amino acid sequence selected from the group consisting of SEQ ID NOs:1-38, and combinations thereof.

In some examples, including any of the foregoing, the method includes fragmenting a glycopeptide in the sample to provide a glycopeptide consisting essentially of an amino acid sequence selected from the group consisting of SEQ ID NOs:1-38, and combinations thereof.

In some examples, including any of the foregoing, the method includes detecting a multiple-reaction-monitoring (MRM) transition selected from the group consisting of transitions 1-38. In some examples, the method includes detecting a MRM transition indicative of a glycopeptide or glycan residue, wherein the glycopeptide consists essentially of an amino acid sequence selected from the group consisting of SEQ ID NOs: 1-38 and combinations thereof. In some examples, the method includes detecting a MRM transition indicative of a glycopeptide consisting essentially of an amino acid sequence selected from the group consisting of SEQ ID NOs: 1-38 and combinations thereof. In some examples, the method includes detecting more than one MRM transition selected from a combination of members from the group consisting of transitions 1-38. In some examples, the method includes detecting more than one MRM transition indicative of a combination of glycopeptides having amino acid sequences selected from a combination of SEQ ID NOs: 1-38.

In some examples, including any of the foregoing, the method includes performing mass spectroscopy on the biological sample using multiple-reaction-monitoring mass spectroscopy (MRM-MS).

In some examples, including any of the foregoing, the method includes digesting a glycopeptide in the sample to provide a glycopeptide consisting essentially of an amino acid sequence selected from the group consisting of SEQ ID NOs:1-38, and combinations thereof. In certain examples, the biological sample is combined with chemical reagents. In certain examples, the biological sample is combined with enzymes. In some examples, the enzymes are lipases. In some examples, the enzymes are proteases. In some examples, the enzymes are serine proteases. In some of these examples, the enzyme is selected from the group consisting of trypsin, chymotrypsin, thrombin, elastase, and subtilisin. In some of these examples, the enzyme is trypsin. In some examples, the methods includes contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, including any of the foregoing, the method includes detecting a multiple-reaction-monitoring (MRM) transition selected from the group consisting of transitions 1-38. In some examples, the method includes detecting a MRM transition indicative of a glycopeptide or glycan residue, wherein the glycopeptide consisting of, or consisting essentially of, an amino acid sequence selected from the group consisting of SEQ ID NOs: 1-38 and combinations thereof. In some examples, the method includes detecting a MRM transition indicative of a glycopeptide or glycan residue, wherein the glycopeptide consists essentially of an amino acid sequence selected from the group consisting of SEQ ID NOs: 1-38 and combinations thereof. In some examples, the method includes detecting a MRM transition indicative of a glycopeptide consisting essentially of an amino acid sequence selected from the group consisting of SEQ ID NOs: 1-38 and combinations thereof. In some examples, the method includes detecting more than one MRM transition selected from a combination of members from the group consisting of transitions 1-38. In some examples, the method includes detecting more than one MRM transition indicative of a combination of glycopeptides having amino acid sequences selected from a combination of SEQ ID NOs: 1-38.

In some examples, including any of the foregoing, the method includes performing mass spectroscopy on the biological sample using multiple-reaction-monitoring mass spectroscopy (MRM-MS).

In some examples, including any of the foregoing, the method includes digesting a glycopeptide in the sample to provide a glycopeptide consisting of an amino acid sequence selected from the group consisting of SEQ ID NOs:1-38, and combinations thereof. In certain examples, the biological sample is contacted with one or more chemical reagents. In certain examples, the biological sample is contacted with one or more enzymes. In some examples, the enzymes are lipases. In some examples, the enzymes are proteases. In some examples, the enzymes are serine proteases. In some of these examples, the enzyme is selected from the group consisting of trypsin, chymotrypsin, thrombin, elastase, and subtilisin. In some of these examples, the enzyme is trypsin. In some examples, the methods includes contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, including any of the foregoing, the MRM transition is selected from the transitions, or any combinations thereof, in any one of Tables 1, 2 or 3.

In some examples, including any of the foregoing, the method includes conducting tandem liquid chromatography-mass spectroscopy on the biological sample.

In some examples, including any of the foregoing, the method includes multiple-reaction-monitoring mass spectroscopy (MRM-MS) mass spectroscopy on the biological sample.

In some examples, including any of the foregoing, the method includes detecting a MRM transition using a triple quadrupole (QQQ) and/or a quadrupole time-of-flight (qTOF) mass spectrometer. In certain examples, the method includes detecting a MRM transition using a QQQ mass spectrometer. In certain other examples, the method includes detecting using a qTOF mass spectrometer. In some examples, a suitable instrument for use with the instant methods is an Agilent 6495B Triple Quadrupole LC/MS, which can be found at www.agilent.com/en/products/mass-spectrometry/lc-ms-instruments/triple-quadrupole-lc-ms/6495b-triple-quadrupole-lc-ms. In certain other examples, the method includes detecting using a QQQ mass spectrometer. In some examples, a suitable instrument for use with the instant methods is an Agilent 6545 LC/Q-TOF, which can be found at https://www.agilent.com/en/products/liquid-chromatography-mass-spectrometry-lc-ms/lc-ms-instruments/quadrupole-time-of-flight-lc-ms/6545-q-tof-lc-ms.

In some examples, including any of the foregoing, the method includes detecting more than one MRM transition using a QQQ and/or qTOF mass spectrometer. In certain examples, the method includes detecting more than one MRM transition using a QQQ mass spectrometer. In certain examples, the method includes detecting more than one MRM transition using a qTOF mass spectrometer. In certain examples, the method includes detecting more than one MRM transition using a QQQ mass spectrometer.

In some examples, including any of the foregoing, the methods herein include quantifying one or more glycomic parameters of the one or more biological samples comprises employing a coupled chromatography procedure. In some examples, these glycomic parameters include the identification of a glycopeptide group, identification of glycans on the glycopeptide, identification of a glycosylation site, identification of part of an amino acid sequence which the glycopeptide includes. In some examples, the coupled chromatography procedure comprises: performing or effectuating a liquid chromatography-mass spectrometry (LC-MS) operation. In some examples, the coupled chromatography procedure comprises: performing or effectuating a multiple reaction monitoring mass spectrometry (MRM-MS) operation. In some examples, the methods herein include a coupled chromatography procedure which comprises: performing or effectuating a liquid chromatography-mass spectrometry (LC-MS) operation; and effectuating a multiple reaction monitoring mass spectrometry (MRM-MS) operation. In some examples, the methods include training a machine learning system using one or more glycomic parameters of the one or more biological samples obtained by one or more of a triple quadrupole (QQQ) mass spectrometry operation and/or a quadrupole time-of-flight (qTOF) mass spectrometry operation. In some examples, the methods include training a machine learning system using one or more glycomic parameters of the one or more biological samples obtained a triple quadrupole (QQQ) mass spectrometry operation. In some examples, the methods include training a machine learning system using one or more glycomic parameters of the one or more biological samples obtained by a quadrupole time-of-flight (qTOF) mass spectrometry operation. In some examples, the methods include quantifying one or more glycomic parameters of the one or more biological samples comprises employing one or more of a triple quadrupole (QQQ) mass spectrometry operation and a quadrupole time-of-flight (qTOF) mass spectrometry operation. In some examples, machine learning systems are used to quantify these glycomic parameters. In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay (e.g., ELISA) is used in combination with mass spectroscopy. In some examples, the immunoassay measures CA-125 and HE4 proteins.

In some examples, including any of the foregoing, the glycopeptide or combination thereof consists of an amino acid sequence selected from the group consisting of SEQ ID NOs:1-38 and combinations thereof.

In some examples, including any of the foregoing, the glycopeptide or combination thereof consists essentially of an amino acid sequence selected from the group consisting of SEQ ID NOs:1-38 and combinations thereof.

In some examples, including any of the foregoing, the method includes digesting and/or fragmenting a glycopeptide in the sample to provide a glycopeptide consisting of an amino acid sequence selected from the group consisting of SEQ ID NOs: 1-38 and combinations thereof.

In some examples, including any of the foregoing, the method includes digesting and/or fragmenting a glycopeptide in the sample to provide a glycopeptide consisting essentially of an amino acid sequence selected from the group consisting of SEQ ID NOs: 1-38 and combinations thereof.

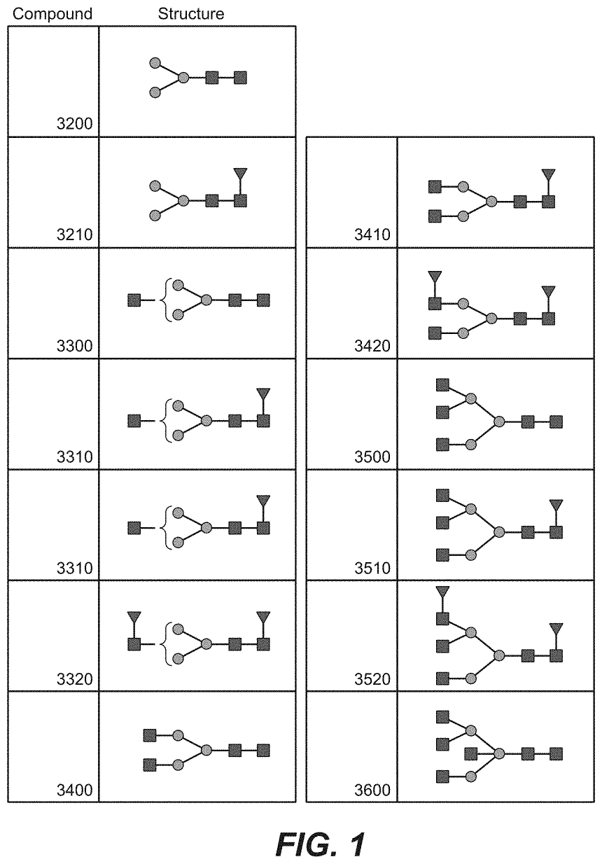

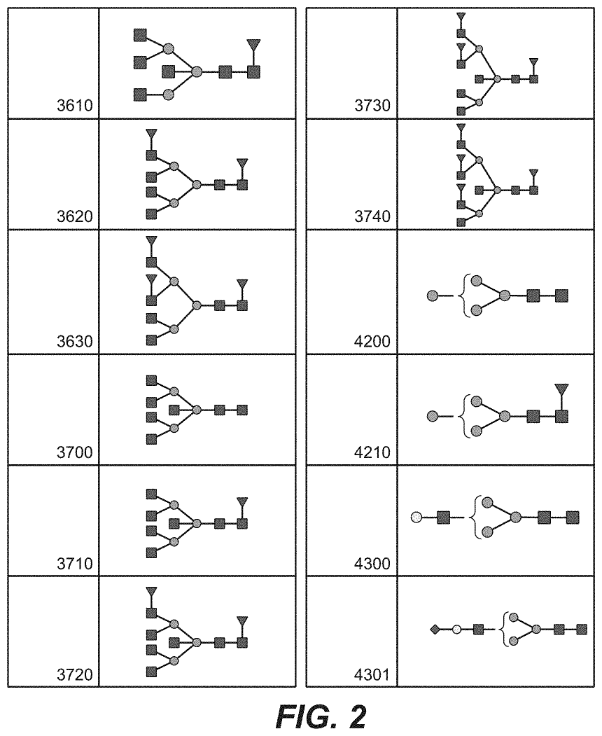

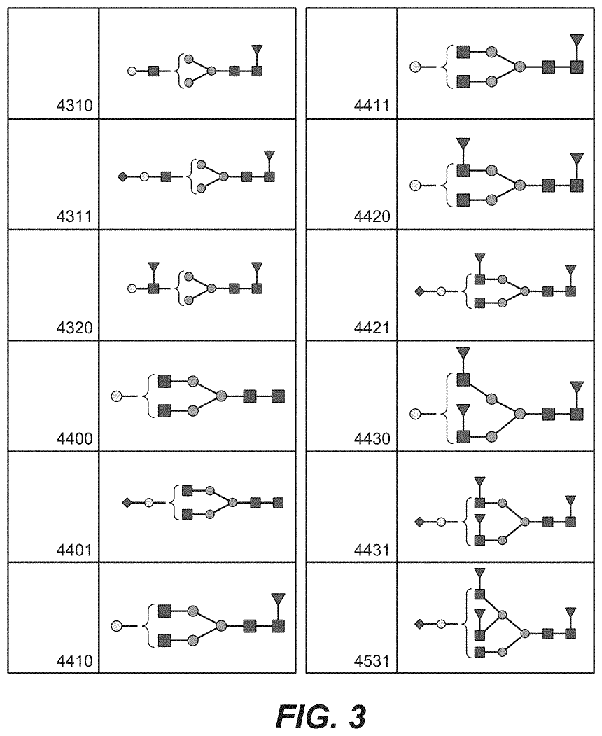

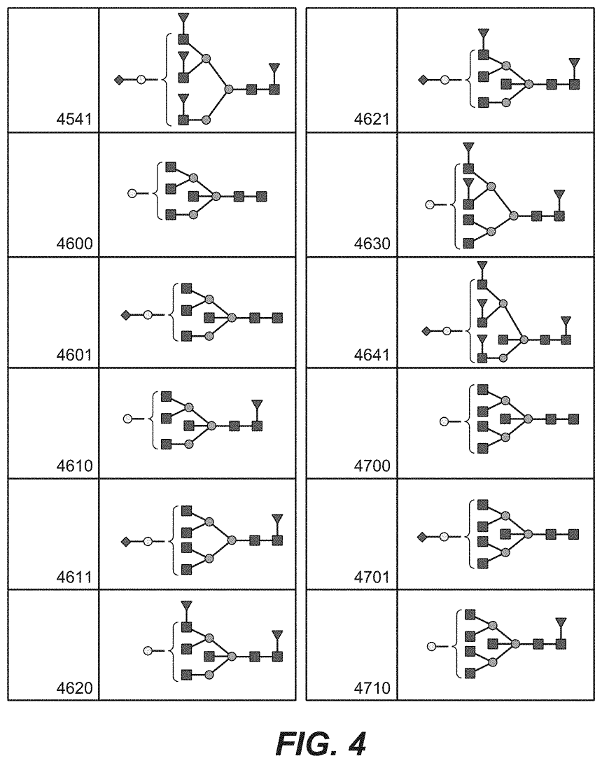

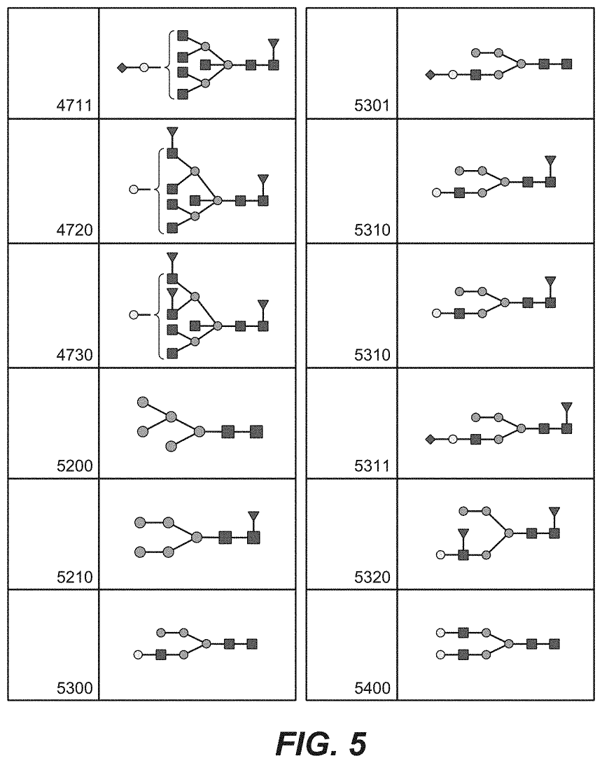

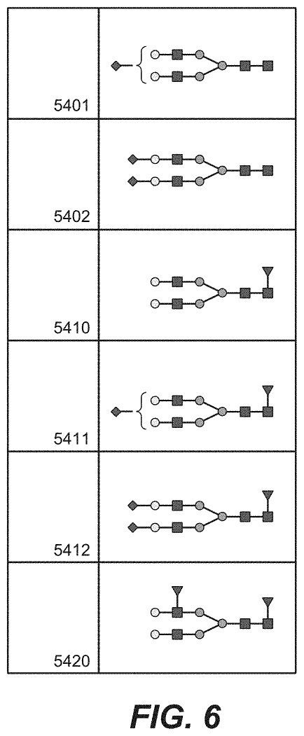

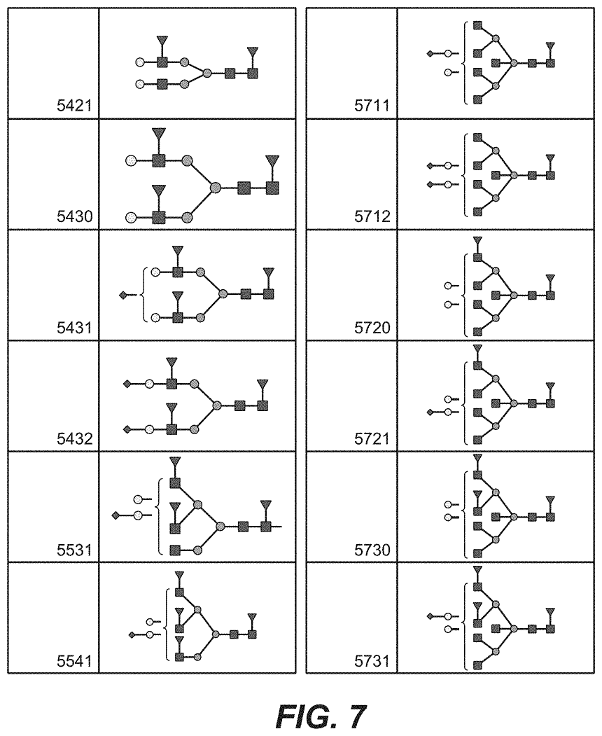

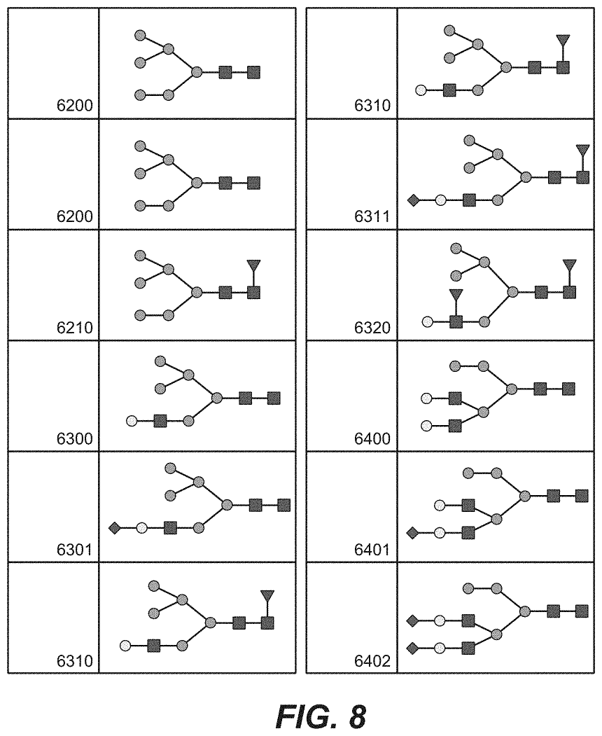

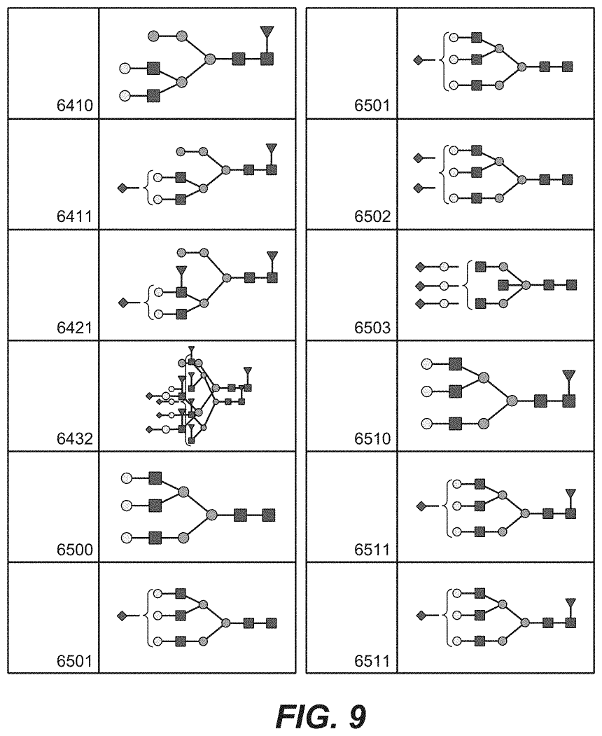

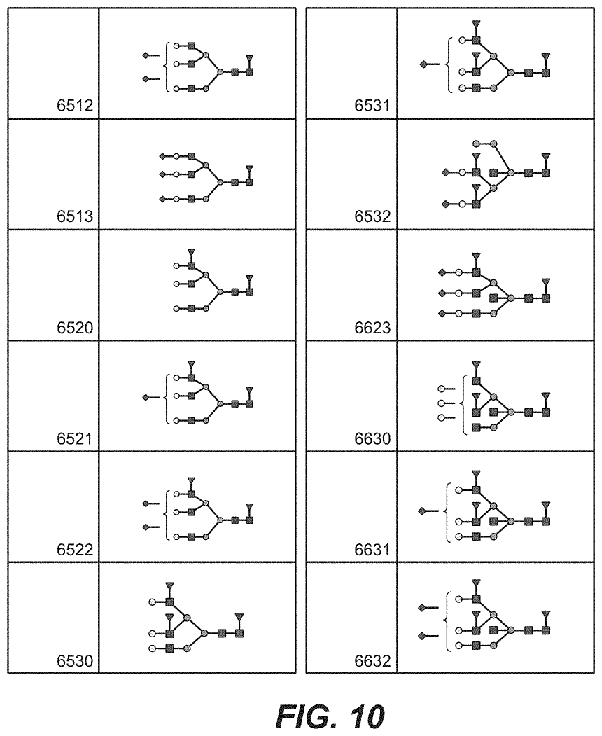

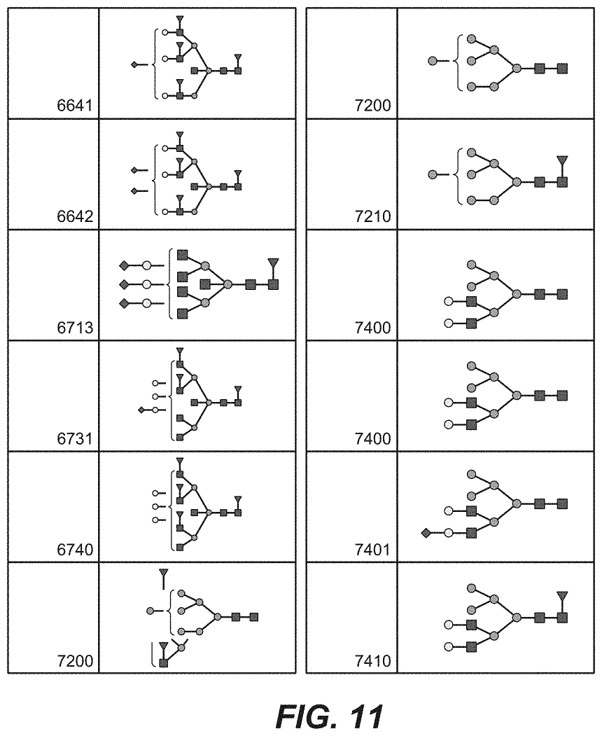

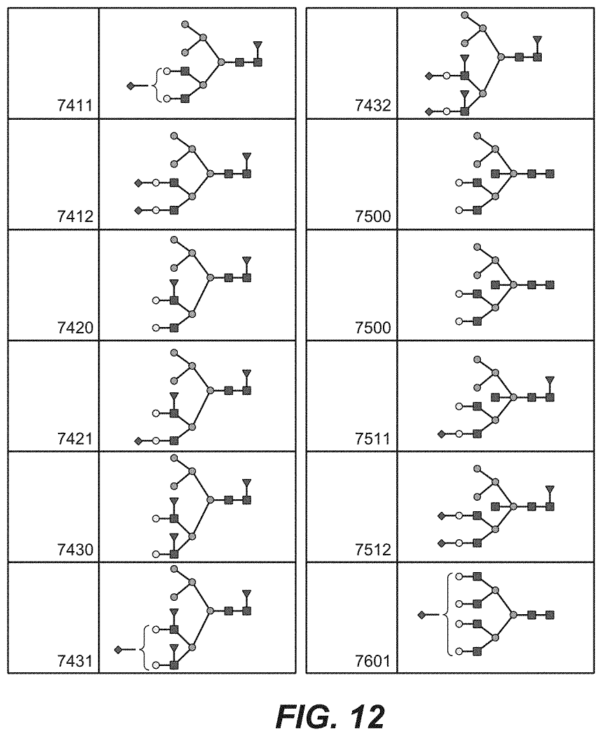

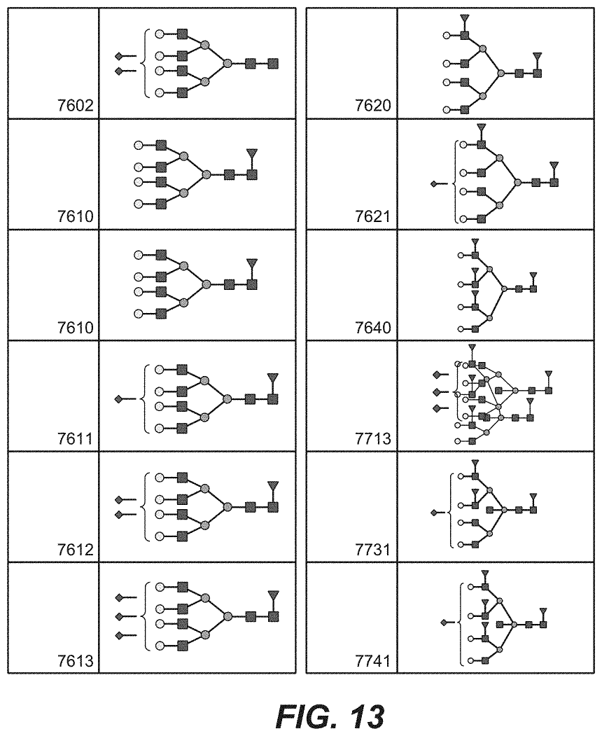

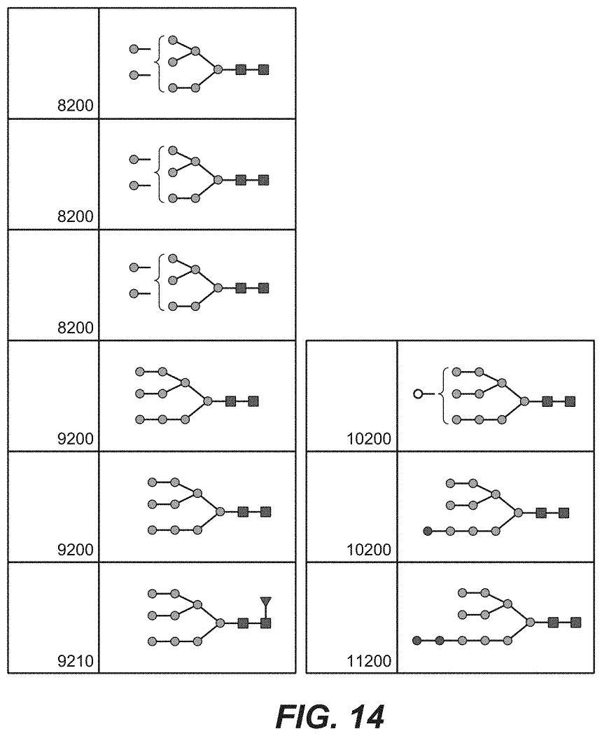

In some examples, including any of the foregoing, the method includes detecting one or more MRM transitions indicative of glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof. Herein, these glycans are illustrated in FIGS. 1-14.

In some examples, including any of the foregoing, the method includes quantifying a glycan.

In some examples, including any of the foregoing, the method includes quantifying a first glycan and quantifying a second glycan; and further comprising comparing the quantification of the first glycan with the quantification of the second glycan.

In some examples, including any of the foregoing, the method includes associating the detected glycan with a peptide residue site, whence the glycan was bonded.

In some examples, including any of the foregoing, the method includes generating a glycosylation profile of the sample.

In some examples, including any of the foregoing, the method includes spatially profiling glycans on a tissue section associated with the sample. In some examples, including any of the foregoing, the method includes spatially profiling glycopeptides on a tissue section associated with the sample. In some examples, the method includes matrix-assisted laser desorption ionization time-of-flight mass spectrometry (MALDI-TOF) mass spectroscopy in combination with the methods herein.

In some examples, including any of the foregoing, the method includes quantifying relative abundance of a glycan and/or a peptide.

In some examples, including any of the foregoing, the method includes normalizing the amount of a glycopeptide by quantifying a glycopeptide consisting essentially of an amino acid sequence selected from the group consisting of SEQ ID NOs:1-38, and combinations thereof and comparing that quantification to the amount of another chemical species. In some examples, the method includes normalizing the amount of a peptide by quantifying a glycopeptide consisting of an amino acid sequence selected from the group consisting of SEQ ID NOs:1-38, and combinations thereof, and comparing that quantification to the amount of another glycopeptide consisting of an amino acid sequence selected from the group consisting of SEQ ID NOs:1-38. In some examples, the method includes normalizing the amount of a peptide by quantifying a glycopeptide consisting essentially of an amino acid sequence selected from the group consisting of SEQ ID NOs:1-38, and combinations thereof, and comparing that quantification to the amount of another glycopeptide consisting essentially of an amino acid sequence selected from the group consisting of SEQ ID NOs:1-38.

B. Methods for Classifying Samples Comprising Glyopeptides

In another embodiment, set forth herein a method for identifying a classification for a sample, the method comprising: quantifying by mass spectroscopy (MS) one or more glycopeptides in a sample wherein the glycopeptides each, individually in each instance, comprises a glycopeptide consisting essentially of an amino acid sequence selected from the group consisting of, or consisting essentially of, SEQ ID NOs:1-38, and combinations thereof; and inputting the quantification into a trained model to generate a output probability; determining if the output probability is above or below a threshold for a classification; and identifying a classification for the sample based on whether the output probability is above or below a threshold for a classification.

In some examples, set forth herein is a method for classifying glycopeptides, comprising: obtaining a biological sample from a patient; digesting and/or fragmenting a glycopeptide in the sample; detecting a multiple-reaction-monitoring (MRM) transition selected from the group consisting of transitions 1-38; and classifying the glycopeptides based on the MRM transitions detected. In some examples, a machine learning system is used to train a model using the analyzed the MRM transitions as inputs. In some examples, a machine learning system is trained using the MRM transitions as a training data set. In some examples, the methods herein include identifying glycopeptides, peptides, and glycans based on their mass spectroscopy relative abundance. In some examples, a machine learning system or systems select and/or identify peaks in a mass spectroscopy spectrum.

In some examples, set forth herein is a method for classifying glycopeptides, comprising: obtaining a biological sample from an individual; digesting and/or fragmenting a glycopeptide in the sample; detecting a multiple-reaction-monitoring (MRM) transition selected from the group consisting of transitions 1-38; and classifying the glycopeptides based on the MRM transitions detected. In some examples, a machine learning system is used to train a model using the analyzed the MRM transitions as inputs. In some examples, a machine learning system is trained using the MRM transitions as a training data set. In some examples, the methods herein include identifying glycopeptides, peptides, and glycans based on their mass spectroscopy relative abundance. In some examples, a machine learning system or systems select and/or identify peaks in a mass spectroscopy spectrum.

In some examples, set forth herein is a method of training a machine learning system using MRM transitions as an input data set. In some examples, set forth herein is a method for identifying a classification for a sample, the method comprising quantifying by mass spectroscopy (MS) a glycopeptide in a sample wherein the glycopeptide consisting of, or consisting essentially of, an amino acid sequence selected from the group consisting of SEQ ID NOs:1-38, and combinations thereof; and identifying a classification based on the quantification. In some examples, the quantifying includes determining the presence or absence of a glycopeptide, or combination of glycopeptides, in a sample. In some examples, the quantifying includes determining the relative abundance of a glycopeptide, or combination of glycopeptides, in a sample. In some examples, the identifying a classification based on quantification can be achieved by selecting any 10, 15, 16, 18, 20, 25, or 30, or any 10-30 of glycopeptide amino acid sequences from the group consisting of SEQ ID Nos: 1-38.

In some examples, including any of the foregoing, the sample is a biological sample from a patient having a disease or condition.

In some examples, including any of the foregoing, the patient has ovarian cancer.

In some examples, including any of the foregoing, the patient has cancer.

In some examples, including any of the foregoing, the patient has fibrosis.

In some examples, including any of the foregoing, the patient has an autoimmune disease.

In some examples, including any of the foregoing, the disease or condition is ovarian cancer.

In some examples, including any of the foregoing, the MS is MRM-MS with a QQQ and/or qTOF mass spectrometer.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay is used in combination with mass spectroscopy. In some examples, the immunoassay measures CA-125 and HE4.

In some examples, including any of the foregoing, the machine learning system is selected from the group consisting of a deep learning system, a neural network system, an artificial neural network system, a supervised machine learning system, a linear discriminant analysis system, a quadratic discriminant analysis system, a support vector machine system, a linear basis function kernel support vector system, a radial basis function kernel support vector system, a random forest system, a genetic algorithm system, a nearest neighbor system, k-nearest neighbors, a naive Bayes classifier system, a logistic regression system, or a combination thereof. In certain examples, the machine learning process is lasso regression.

In some examples, including any of the foregoing, the method includes classifying a sample as within, or embraced by, a disease classification or a disease severity classification.

In some examples, including any of the foregoing, the classification is identified with 80% confidence, 85% confidence, 90% confidence, 95% confidence, 99% confidence, or 99.9999% confidence.

In some examples, including any of the foregoing, the method includes quantifying by MS the glycopeptide in a sample at a first time point; quantifying by MS the glycopeptide in a sample at a second time point; and comparing the quantification at the first time point with the quantification at the second time point.

In some examples, including any of the foregoing, the method includes quantifying by MS a different glycopeptide in a sample at a third time point; quantifying by MS the different glycopeptide in a sample at a fourth time point; and comparing the quantification at the fourth time point with the quantification at the third time point.

In some examples, including any of the foregoing, the method includes monitoring the health status of a patient.

In some examples, including any of the foregoing, monitoring the health status of a patient includes monitoring the onset and progression of disease in a patient with risk factors such as genetic mutations, as well as detecting cancer recurrence.

In some examples, including any of the foregoing, the method includes quantifying by MS a glycopeptide consisting of an amino acid sequence selected from the group consisting of SEQ ID NOs:1-38.

In some examples, including any of the foregoing, the method includes quantifying by MS a glycopeptide consisting essentially of an amino acid sequence selected from the group consisting of SEQ ID NOs:1-38.

In some examples, including any of the forgoing, the method includes quantifying by MS a set of any 10, 15, 16, 18, 20, 25, or 30, or any number between 10-30 of glycopeptides to classify a sample as within, or embraced by, a disease classification or a disease severity classification; e.g. ovarian cancer.

In some examples, including any of the foregoing, the method includes quantifying by MS one or more glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof. Herein, these glycans are illustrated in FIGS. 1-14.

In some examples, including any of the foregoing, the method includes diagnosing a patient with a disease or condition based on the quantification.

In some examples, including any of the foregoing, the method includes diagnosing the patient as having ovarian cancer based on the quantification.

In some examples, including any of the foregoing, the method includes treating the patient with a therapeutically effective amount of a therapeutic agent selected from the group consisting of a chemotherapeutic, an immunotherapy, a hormone therapy, a targeted therapy, a neoadjuvant therapy, surgery, and combinations thereof.

In some examples, including any of the foregoing, the method includes diagnosing an individual with a disease or condition based on the quantification.

In some examples, including any of the foregoing, the method includes diagnosing the individual as having an aging condition.

In some examples, including any of the foregoing, the method includes treating the individual with a therapeutically effective amount of an anti-aging agent. In some examples, the anti-aging agent is selected from hormone therapy. In some examples, the anti-aging agent is testosterone or a testosterone supplement or derivative. In some examples, the anti-aging agent is estrogen or an estrogen supplement or derivative.

C. Methods of Treatment

In some examples, set forth herein is a method for treating a patient having a disease or condition, comprising measuring by mass spectroscopy a glycopeptide in a sample from the patient. In some examples, the patient is a human. In certain examples, the patient is a female. In certain other examples, the patient is a female with ovarian cancer. In certain examples, the patient is a female with ovarian cancer at Stage 1. In certain examples, the patient is a female with ovarian cancer at Stage 2. In certain examples, the patient is a female with ovarian cancer at Stage 3. In certain examples, the patient is a female with ovarian cancer at Stage 4. In some examples, the female has an age equal or between 10-20 years. In some examples, the female has an age equal or between 20-30 years. In some examples, the female has an age equal or between 30-40 years. In some examples, the female has an age equal or between 40-50 years. In some examples, the female has an age equal or between 50-60 years. In some examples, the female has an age equal or between 60-70 years. In some examples, the female has an age equal or between 70-80 years. In some examples, the female has an age equal or between 80-90 years. In some examples, the female has an age equal or between 90-100 years.

In another embodiment, set forth herein is a method for treating a patient having ovarian cancer; the method comprising: obtaining a biological sample from the patient; digesting and/or fragmenting one or more glycopeptides in the sample; and detecting and quantifying one or more multiple-reaction-monitoring (MRM) transitions selected from the group consisting of transitions 1-38; inputting the quantification into a trained model to generate an output probability; determining if the output probability is above or below a threshold for a classification; and classifying the patient based on whether the output probability is above or below a threshold for a classification, wherein the classification is selected from the group consisting of: (A) a patient in need of a chemotherapeutic agent; (B) a patient in need of a immunotherapeutic agent; (C) a patient in need of hormone therapy; (D) a patient in need of a targeted therapeutic agent; (E) a patient in need of surgery; (F) a patient in need of neoadjuvant therapy; (G) a patient in need of chemotherapeutic agent, immunotherapeutic agent, hormone therapy, targeted therapeutic agent, neoadjuvant therapy, or a combination thereof, before surgery; (H) a patient in need of chemotherapeutic agent, immunotherapeutic agent, hormone therapy, targeted therapeutic agent, neoadjuvant therapy, or a combination thereof, after surgery; (I) or a combination thereof; administering a therapeutically effective amount of a therapeutic agent to the patient: wherein the therapeutic agent is selected from chemotherapy if classification A or I is determined; wherein the therapeutic agent is selected from immunotherapy if classification B or I is determined; or wherein the therapeutic agent is selected from hormone therapy if classification C or I is determined; or wherein the therapeutic agent is selected from targeted therapy if classification D or I is determined wherein the therapeutic agent is selected from neoadjuvant therapy if classification F or I is determined; wherein the therapeutic agent is selected from chemotherapeutic agent, immunotherapeutic agent, hormone therapy, targeted therapeutic agent, neoadjuvant therapy, or a combination thereof if classification G or I is determined; and wherein the therapeutic agent is selected from chemotherapeutic agent, immunotherapeutic agent, hormone therapy, targeted therapeutic agent, neoadjuvant therapy, or a combination thereof if classification H or I is determined.

In some examples, the machine learning is used to identify MS peaks associated with MRM transitions. In some examples, the MRM transitions are analyzed using machine learning. In some examples, the machine learning is used to train a model based on the quantification of the amount of glycopeptides associated with an MRM transition(s). In some examples, the MRM transitions are analyzed with a trained machine learning system. In some of these examples, the trained machine learning system was trained using MRM transitions observed by analyzing samples from patients known to have ovarian cancer.

In some examples, the patient is treated with a therapeutic agent selected from targeted therapy. In some examples, the methods herein include administering a therapeutically effective amount of a (poly(ADP)-ribose polymerase) (PARP) inhibitor if combination D is detected. In some examples, the therapeutic agent is selected from Olaparib (Lynparza), Rucaparib (Rubraca), and Niraparib (Zejula).

In some examples, the patient is an adult with platinum-sensitive relapsed high-grade epithelial ovarian, fallopian tube, or primary peritoneal cancer.

In some examples, the therapeutic agent is administered at 150 mg, 250 mg, 300 mg, 350 mg, and 600 mg doses. In some examples, the therapeutic agent is administered twice daily.

Chemotherapeutic agents include, but are not limited to, platinum-based drug such as carboplatin (Paraplatin) or cisplatin with a taxane such as paclitaxel (Taxol) or docetaxel (Taxotere). Paraplatin may be administered at 10 mg/mL injectable concentrations (in vials of 50, 150, 450, and 600 mg). For advanced ovarian carcinoma a single agent dose of 360 mg/m² IV for 4 weeks may be administered. Paraplatin may be administered in combination=as 300 mg/m² IV (plus cyclophosphamide 600 mg/m² IV) q4Weeks. Taxol may be administered at 175 mg/m² IV over 3 hours q3Weeks (follow with cisplatin). Taxol may be administered at 135 mg/m² IV over 24 hours q3Weeks (follow with cisplatin). Taxol may be administered at 135-175 mg/m² IV over 3 hours q3Weeks.

Immunotherapeutic agents include, but are not limited to, Zejula (Niraparib). Niraparib may be administered at 300 mg PO qDay.

Hormone therapeutic agents include, but are not limited to, Luteinizing-hormone-releasing hormone (LHRH) agonists, Tamoxifen, and Aromatase inhibitors.

Targeted therapeutic agents include, but are not limited to, PARP inhibitors.

In some examples, including any of the foregoing, the method includes conducting multiple-reaction-monitoring mass spectroscopy (MRM-MS) on the biological sample.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay (e.g., ELISA) is used in combination with mass spectroscopy. In some examples, the immunoassay measures CA-125 and HE4.

In some examples, including any of the foregoing, the method includes quantifying one or more glycopeptides consisting of an amino acid sequence selected from the group consisting of SEQ ID NOs:1-38 and combinations thereof.

In some examples, including any of the foregoing, the method includes quantifying one or more glycopeptides consisting essentially of an amino acid sequence selected from the group consisting of SEQ ID NOs:1-38 and combinations thereof.

In some examples, including any of the foregoing, the method includes detecting a multiple-reaction-monitoring (MRM) transition selected from the group consisting of transitions 1-38 using a QQQ and/or a qTOF mass spectrometer.

In some examples, including any of the foregoing, the method includes training a machine learning system to identify a classification based on the quantifying step.

In some examples, including any of the foregoing, the method includes using a machine learning system to identify a classification based on the quantifying step.

In some examples, including any of the foregoing, the machine learning system is selected from the group consisting of a deep learning system, a neural network system, an artificial neural network system, a supervised machine learning system, a linear discriminant analysis system, a quadratic discriminant analysis system, a support vector machine system, a linear basis function kernel support vector system, a radial basis function kernel support vector system, a random forest system, a genetic system, a nearest neighbor system, k-nearest neighbors, a naive Bayes classifier system, a logistic regression system, or a combination thereof.

D. Methods for Diagnosing Patients

In some examples, set forth herein is a method for diagnosing a patient having a disease or condition, comprising measuring by mass spectroscopy a glycopeptide in a sample from the patient.

In another embodiment, set forth herein is a method for diagnosing a patient having ovarian cancer; the method comprising: obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs:1-38; or to detect and quantify one or more MRM transitions selected from transitions 1-38; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having ovarian cancer based on the diagnostic classification.

In another embodiment, set forth herein is a method for diagnosing a patient having ovarian cancer; the method comprising: inputting the quantification of detected glycopeptides or MRM transitions into a trained model to generate an output probability, determining if the output probability is above or below a threshold for a classification; and identifying a diagnostic classification for the patient based on whether the output probability is above or below a threshold for a classification; and diagnosing the patient as having ovarian cancer based on the diagnostic classification. In some examples, the method includes obtaining a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs:1-38; or to detect and quantify one or more MRM transitions selected from transitions 1-38.

In some examples, set forth herein is a method for diagnosing a patient having ovarian cancer; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs:1-38; or to detect one or more MRM transitions selected from transitions 1-38; analyzing the detected glycopeptides or the MRM transitions to identify a diagnostic classification; and diagnosing the patient as having ovarian cancer based on the diagnostic classification. In some examples, the method includes obtaining a biological sample from the patient; and performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs:1-38; or to detect one or more MRM transitions selected from transitions 1-38.

In some examples, set forth herein is a method for diagnosing, monitoring, or classifying aging in an individual; the method comprising: obtaining a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs:1-38; or to detect one or more MRM transitions selected from transitions 1-38; analyzing the detected glycopeptides or the MRM transitions to identify a diagnostic classification; and diagnosing, monitoring, or classifying the individual as having an aging classification based on the diagnostic classification.

E. Diseases and Conditions

Set forth herein are biomarkers for diagnosing a variety of diseases and conditions.

In some examples, the diseases and conditions include cancer. In some examples, the diseases and conditions are not limited to cancer.

In some examples, the diseases and conditions include fibrosis. In some examples, the diseases and conditions are not limited to fibrosis.

In some examples, the diseases and conditions include an autoimmune disease. In some examples, the diseases and conditions are not limited to an autoimmune disease.

In some examples, the diseases and conditions include ovarian cancer. In some examples, the diseases and conditions are not limited to ovarian cancer.

In some examples, the condition is aging. In some examples, the “patient” described herein is equivalently described as an “individual.” For example, in some methods herein, set forth are biomarkers for monitoring or diagnosing aging or aging conditions in an individual. In some of these examples, the individual is not necessarily a patient who has a medical condition in need of therapy. In some examples, the individual is a male. In some examples, the individual is a female. In some examples, the individual is a male mammal. In some examples, the individual is a female mammal. In some examples, the individual is a male human. In some examples, the individual is a female human.

In some examples, the individual is between 1 years old and 100 years old, or any number inbetween.

### IV. MACHINE LEARNING

In some examples, including any of the foregoing, the methods herein include quantifying one or more glycopeptides consisting essentially of an amino acid sequence selected from the group consisting of SEQ ID NOs:1-38 using mass spectroscopy and/or liquid chromatography. In some examples, the quantification results are used as inputs in a trained model. In some examples, the quantification results are classified or categorized with a diagnostic system based on the absolute amount, relative amount, and/or type of each glycan or glycopeptide quantified in the test sample, wherein the diagnostic system is trained on corresponding values for each marker obtained from a population of individuals having known diseases or conditions. In some examples, the disease or condition is ovarian cancer.

In some examples, including any of the foregoing, set forth herein is a method for training a machine learning system, comprising: providing a first data set of MRM transition signals indicative of a sample comprising a glycopeptide consisting essentially of an amino acid sequence selected from the group consisting of SEQ ID NOs: 1-38; providing a second data set of MRM transition signals indicative of a control sample; and comparing the first data set with the second data set using a machine learning system.

In some examples, including any of the foregoing, the method herein include using a sample comprising a glycopeptide consisting of an amino acid sequence selected from the group consisting of SEQ ID NOs: 1-38 is a sample from a patient having ovarian cancer.

In some examples, including any of the foregoing, the method herein include using a sample comprising a glycopeptide consisting essentially of an amino acid sequence selected from the group consisting of SEQ ID NOs: 1-38 is a sample from a patient having ovarian cancer.

In some examples, including any of the foregoing, the method herein include using a control sample, wherein the control sample is a sample from a patient not having ovarian cancer.

In some examples, including any of the foregoing, the method herein include using a sample comprising a glycopeptide consisting essentially of an amino acid sequence selected from the group consisting of SEQ ID NOs: 1-38, which is a pooled sample from one or more patients having ovarian cancer.

In some examples, including any of the foregoing, the method herein include using a control sample, which is a pooled sample from one or more patients not having ovarian cancer.

In some examples, including any of the foregoing, the methods include generating machine learning models trained using mass spectrometry data (e.g., MRM-MS transition signals) from patients having a disease or condition and patients not having a disease or condition. In some examples, the disease or condition is ovarian cancer. In some examples, the methods include optimizing the machine learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis. In some examples, one to ten glycopeptides consisting essentially of an amino acid sequence selected from the group consisting of SEQ ID NOs:1-38 may be useful for diagnosing a patient with ovarian cancer with a certain confidence percent. In some examples, ten to fifty glycopeptides consisting essentially of an amino acid sequence selected from the group consisting of SEQ ID NOs:1-38 may be useful for diagnosing a patient with ovarian cancer with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NOs:1-38. In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, including any of the foregoing, the methods include generating machine learning models trained using mass spectrometry data (e.g., MRM-MS transition signals) from patients having a disease or condition and patients not having a disease or condition. In some examples, the disease or condition is ovarian cancer. In some examples, the methods include optimizing the machine learning models by cross-validation with known standards or other samples. In some examples, the methods include qualifying the performance using the mass spectrometry data to form panels of glycans and glycopeptides with individual sensitivities and specificities.

In some examples, machine learning systems are used to determine, by the computing device and based on the MRM-MS data, a distribution of a plurality of characteristic ions in the plurality of mass spectra; and determining, by the computing device and based on the distribution, whether one or more of the plurality of characteristic ions is a glycopeptide ion.

In some examples, the methods herein include training a diagnostic system. Herein, training the diagnostic system may refer to supervised learning of a diagnostic system on the basis of values for one or more glycopeptides consisting of, or consisting essentially of, an amino acid sequence selected from the group consisting of SEQ ID NOs:1-38. Training the diagnostic system may refer to variable selection in a statistical model on the basis of values for one or more glycopeptides consisting essentially of an amino acid sequence selected from the group consisting of SEQ ID NOs:1-38. Training a diagnostic system may for example include determining a weighting vector in feature space for each category, or determining a function or function parameters.

In some examples, including any of the foregoing, the machine learning system is selected from the group consisting of a deep learning system, a neural network system, an artificial neural network system, a supervised machine learning system, a linear discriminant analysis system, a quadratic discriminant analysis system, a support vector machine system, a linear basis function kernel support vector system, a radial basis function kernel support vector system, a random forest system, a genetic system, a nearest neighbor system, k-nearest neighbors, a naive Bayes classifier system, a logistic regression system, or a combination thereof. In certain examples, the machine learning system is lasso regression.

In certain examples, the machine learning system uses a process selected from the following: LASSO, Ridge Regression, Random Forests, K-nearest Neighbors (KNN), Deep Neural Networks (DNN), and Principal Components Analysis (PCA). In certain examples, DNN's are used to process mass spectrometry data into analysis-ready forms. In some examples, DNN's are used for peak picking from a mass spectra. In some examples, PCA is useful in feature detection.

In some examples, LASSO is used to provide feature selection.

In some examples, machine learning systems are used to quantify peptides from each protein that are representative of the protein abundance. In some examples, this quantification includes quantifying proteins for which glycosylation is not measured.

In some examples, glycopeptide sequences are identified by fragmentation in the mass spectrometer and database search using Byonic software.

In some examples, the methods herein include unsupervised learning to detect features of MRMS-MS data that represent known biological quantities, such as protein function or glycan motifs. In certain examples, these features are used as input for classifying by machine. In some examples, the classification is performed using LASSO, Ridge Regression, or Random Forest nature.

In some examples, the methods herein include mapping input data (e.g., MRM transition peaks) to a value (e.g., a scale based on 0-100) before processing the value in a trained system. For example, after a MRM transition is identified and the peak characterized, the methods herein include assessing the MS scans in an m/z and retention time window around the peak for a given patient. In some examples, the resulting chromatogram is integrated by a machine learning system that determines the peak start and stop points, and calculates the area bounded by those points and the intensity (height). The resulting integrated value is the abundance, which then feeds into machine learning and statistical analyses training and data sets.

In some examples, machine learning output, in one instance, is used as machine learning input in another instance. For example, in addition to the PCA being used for a classification process, the DNN data processing feeds into PCA and other analyses. This results in at least three levels of systemic processing. Other hierarchical structures are contemplated within the scope of the instant disclosure.

In some examples, including any of the foregoing, the methods include comparing the amount of each glycan or glycopeptide quantified in the sample to corresponding reference values for each glycan or glycopeptide in a diagnostic system. In some examples, the methods includes a comparative process by which the amount of a glycan or glycopeptide quantified in the sample is compared to a reference value for the same glycan or glycopeptide using a diagnostic system. The comparative process may be part of a classification by a diagnostic system. The comparative process may occur at an abstract level, e.g., in n-dimensional feature space or in a higher dimensional space.

In some examples, the methods herein include classifying a patient's sample based on the amount of each glycan or glycopeptide quantified in the sample with a diagnostic system. In some examples, the methods include using statistical or machine learning classification processes by which the amount of a glycan or glycopeptide quantified in the test sample is used to determine a category of health with a diagnostic system. In some examples, the diagnostic system is a statistical or machine learning classification system.

In some examples, including any of the foregoing, classification by a diagnostic system may include scoring likelihood of a panel of glycan or glycopeptide values belonging to each possible category, and determining the highest-scoring category. Classification by a diagnostic system may include comparing a panel of marker values to previous observations by means of a distance function. Examples of diagnostic systems suitable for classification include random forests, support vector machines, logistic regression (e.g. multiclass or multinomial logistic regression, and/or systems adapted for sparse logistic regression). A wide variety of other diagnostic systems that are suitable for classification may be used, as known to a person skilled in the art.

In some examples, the methods herein include supervised learning of a diagnostic system on the basis of values for each glycan or glycopeptide obtained from a population of individuals having a disease or condition (e.g., ovarian cancer). In some examples, the methods include variable selection in a statistical model on the basis of values for each glycan or glycopeptide obtained from a population of individuals having ovarian cancer. Training a diagnostic system may for example include determining a weighting vector in feature space for each category, or determining a function or function parameters.

In one embodiment, the reference value is the amount of a glycan or glycopeptide in a sample or samples derived from one individual. Alternatively, the reference value may be derived by pooling data obtained from multiple individuals, and calculating an average (for example, mean or median) amount for a glycan or glycopeptide. Thus, the reference value may reflect the average amount of a glycan or glycopeptide in multiple individuals. Said amounts may be expressed in absolute or relative terms, in the same manner as described herein.

In some examples, the reference value may be derived from the same sample as the sample that is being tested, thus allowing for an appropriate comparison between the two. For example, if the sample is derived from urine, the reference value is also derived from urine. In some examples, if the sample is a blood sample (e.g. a plasma or a serum sample), then the reference value will also be a blood sample (e.g. a plasma sample or a serum sample, as appropriate). When comparing between the sample and the reference value, the way in which the amounts are expressed is matched between the sample and the reference value. Thus, an absolute amount can be compared with an absolute amount, and a relative amount can be compared with a relative amount. Similarly, the way in which the amounts are expressed for classification with the diagnostic system is matched to the way in which the amounts are expressed for training the diagnostic system.

When the amounts of the glycan or glycopeptide are determined, the method may comprise comparing the amount of each glycan or glycopeptide to its corresponding reference value. When the cumulative amount of one, some or all the glycan or glycopeptides are determined, the method may comprise comparing the cumulative amount to a corresponding reference value. When the amounts of the glycan or glycopeptides are combined with each other in a formula to form an index value, the index value can be compared to a corresponding reference index value derived in the same manner.

The reference values may be obtained either within (i.e., constituting a step of) or external to the (i.e., not constituting a step of) methods described herein. In some examples, the methods include a step of establishing a reference value for the quantity of the markers. In other examples, the reference values are obtained externally to the method described herein and accessed during the comparison step of the invention.

In some examples, including any of the foregoing, training of a diagnostic system may be obtained either within (i.e., constituting a step of) or external to (i.e., not constituting a step of) the methods set forth herein. In some examples, the methods include a step of training of a diagnostic system. In some examples, the diagnostic system is trained externally to the method herein and accessed during the classification step of the invention. The reference value may be determined by quantifying the amount of a glycan or glycopeptide in a sample obtained from a population of healthy individual(s). The diagnostic system may be trained by quantifying the amount of a glycan or glycopeptide in a sample obtained from a population of healthy individual(s). As used herein, the term “healthy individual” refers to an individual or group of individuals who are in a healthy state, e.g., patients who have not shown any symptoms of the disease, have not been diagnosed with the disease and/or are not likely to develop the disease. Preferably said healthy individual(s) is not on medication affecting the disease and has not been diagnosed with any other disease. The one or more healthy individuals may have a similar sex, age and body mass index (BMI) as compared with the test individual. The reference value may be determined by quantifying the amount of a glycan or glycopeptide in a sample obtained from a population of individual(s) suffering from the disease. The diagnostic system may be trained by quantifying the amount of a marker in a sample obtained from a population of individual(s) suffering from the disease. More preferably such individual(s) may have similar sex, age and body mass index (BMI) as compared with the test individual. The reference value may be obtained from a population of individuals suffering from ovarian cancer. The diagnostic system may be trained by quantifying the amount of a glycan or glycopeptide in a sample obtained from a population of individuals suffering from ovarian cancer. Once the characteristic glycan or glycopeptide profile of ovarian cancer is determined, the profile of markers from a biological sample obtained from an individual may be compared to this reference profile to determine whether the test subject also has ovarian cancer. Once the diagnostic system is trained to classify ovarian cancer, the profile of markers from a biological sample obtained from an individual may be classified by the diagnostic system to determine whether the test subject is also at that particular stage of ovarian cancer.

### V. Kits

In some examples, including any of the foregoing, set forth herein is a kit comprising a glycopeptide standard, a buffer, and one or more glycopeptides consisting of an amino acid sequence selected from the group consisting of SEQ ID NOs:1-38.

In some examples, including any of the foregoing, set forth herein is a kit comprising a glycopeptide standard, a buffer, and one or more glycopeptides consisting essentially of an amino acid sequence selected from the group consisting of SEQ ID NOs:1-38.

In some examples, including any of the foregoing, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting of SEQ ID NOs:1-38. In some examples, the kit comprises one or more glycopeptides consisting essentially of an amino acid sequence selected from the group consisting of SEQ ID NOs:1-38.

In some examples, including any of the foregoing, set forth herein is a kit comprising the reagents for quantification of the oxidised, nitrated, and/or glycated free adducts derived from glycopeptides.

### VI. Clinical Assays

In some examples, including any of the foregoing, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NOs:1-38. These standards may include one or more glycopeptides consisting essentially of an amino acid sequence selected from the group consisting of SEQ ID NOs:1-38.

In a clinical setting, samples may be prepared (e.g., by digestion) to include one or more glycopeptides consisting of an amino acid sequence selected from the group consisting of SEQ ID NOs:1-38.

In a clinical setting, samples may be prepared (e.g., by digestion) to include one or more glycopeptides consisting essentially of an amino acid sequence selected from the group consisting of SEQ ID NOs:1-38.

In some examples, the amount of a glycan or glycopeptide may be assessed by comparing the amount of one or more glycopeptides consisting of an amino acid sequence selected from the group consisting of SEQ ID NOs:1-38 to the concentration of another biomarker.

In some examples, the amount of a glycan or glycopeptide may be assessed by comparing the amount of one or more glycopeptides consisting essentially of an amino acid sequence selected from the group consisting of SEQ ID NOs:1-38 to the concentration of another biomarker.

In some examples, the amount of a glycan or glycopeptide may be assessed by comparing the amount of one or more glycopeptides consisting of an amino acid sequence selected from the group consisting of SEQ ID NOs:1-38 to the amount of one or more glycopeptides consisting of an amino acid sequence selected from the group consisting of SEQ ID NOs:1-38.

In some examples, the amount of a glycan or glycopeptide may be assessed by comparing the amount of one or more glycopeptides consisting essentially of an amino acid sequence selected from the group consisting of SEQ ID NOs:1-38 to the amount of one or more glycopeptides consisting essentially of an amino acid sequence selected from the group consisting of SEQ ID NOs:1-38.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NOs:1-38.

In some examples, including any of the foregoing, the kit may include software for quantifying the relative amount of a glycopeptide consisting of, or consisting essentially of, an amino acid sequence selected from the group consisting of SEQ ID NOs:1-38.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NOs:1-38 from a patient's sample into a trained model which are stored on a server. In some examples, the server is accessed by the internet, wireless communication, or other digital or telecommunication methods.

In some examples, including any of the foregoing, MRM transition signals 1-38 are stored on a server which is accessed by a clinician performing a method, set forth herein. In some examples, the clinician compares the MRM transition signals from a patient's sample to the MRM transition signals 1-38 which are stored on a server. In some examples, the server is accessed by the internet, wireless communication, or other digital or telecommunication methods.

In some examples, including any of the foregoing, a machine learning system, which has been trained using the MRM transition signals 38, described herein, is stored on a server which is accessed by a clinician performing a method, set forth herein. In some examples, the machine learning system, accessed remotely on a server, analyzes the MRM transition signals from a patient's sample. In some examples, the server is accessed by the internet, wireless communication, or other digital or telecommunication methods.

In some examples, including any of the foregoing, the kit may include software for computing the normalization of a glycopeptide MRM transition signal.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host—pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to diagnose certain diseases, such as ovarian cancer, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., an ovarian cancer disease state). This information can be used to distinguish the disease state from other states, diagnose a subject as having or not having the disease state, determine a likelihood that a subject has the disease state, or a combination thereof. For example, such analysis may be useful in diagnosing an ovarian cancer disease state for a subject (e.g., a negative diagnosis for the ovarian cancer disease state or a positive diagnosis for the ovarian cancer disease state). Sample collection and analysis can be collected at different time points for comparing ovarian cancer disease states over time for a subject. For example, the negative diagnosis may include a healthy state or a benign tumor state (i.e. “benign” as seen throughout). An example of the positive diagnosis includes the subject suffering from a form of ovarian cancer (e.g., epithelial ovarian cancer (EOC)). A diagnosis can also assess a malignancy status of a previously identified pelvic (or adnexal) tumor (or mass).

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing an ovarian cancer disease state. An ovarian cancer disease state may include any condition that can be diagnosed as cancer that occurs in in the ovaries. Many malignant pelvic tumors are ovarian cancer. Certain peptide structures that are associated with an ovarian cancer disease state may be more relevant to that disease state than other peptide structures that are also associated with that disease state.

Analyzing the abundance of peptide sequences and glycosylated peptide sequences in a biological sample may provide a more accurate way in which to distinguish a positive ovarian cancer disease state (e.g., a state including the presence of ovarian cancer) from a negative ovarian cancer disease state (e.g., healthy state, a benign tumor state, an absence of ovarian cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

Further, the methods, systems, and compositions provided by the embodiments described herein may enable an earlier and more accurate diagnosis of ovarian cancer in a subject as compared to currently available diagnostic modalities (e.g., imaging, biochemical tests) used for determining whether surgical intervention is indicated. For example, various currently available non-invasive tests to distinguish between benign and malignant pelvic tumors rely on detection of the biomarker cancer antigen 125 (CA125). But this biomarker is limited by poor sensitivity and specificity. In fact, serum CA125 is not elevated in over 20% of ovarian carcinomas and is elevated in a variety of other malignant and non-malignant conditions. While various other tests incorporate other protein biomarkers in addition to CA125, these other tests may perform less adequately than desired and may be more complex than desired. The embodiments described herein enable more reliable prediction of the malignant or benign nature of pelvic (or adnexal) tumors (or masses)

The description below provides exemplary implementations of the methods and systems described herein for the research, diagnosis, and/or treatment of an ovarian cancer disease state. Various examples implement the methods and systems described herein as a screening tool. Descriptions and examples of various terms, as used herein, are provided in Section II below.

### I. Exemplary Descriptions of Terms

As used herein, the singular forms “a,” “an” and “the” include plural referents unless the context clearly dictates otherwise.

As used herein, the phrase “biological sample,” refers to a sample derived from, obtained by, generated from, provided from, take from, or removed from an organism; or from fluid or tissue from the organism. Biological samples include, but are not limited to synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, any derivative or fraction of the aforementioned biological samples.

As used herein, the term “glycan” refers to the carbohydrate residue of a glycoconjugate, such as the carbohydrate portion of a glycopeptide, glycoprotein, glycolipid or proteoglycan. Glycan structures are described by a glycan reference code number, and also illustrated in International PCT Patent Application No. PCT/US2020/016286, filed Jan. 31, 2020, which is herein incorporated by reference in its entirety for all purposes. For example see FIGS. 1 through 14 of PCT Patent Application No. PCT/US2020/016286, filed Jan. 31, 2020, which are herein incorporated by reference in their entirety for all purposes. Glycans are illustrated using the Symbol Nomenclature for Glycans (SNFG) for illustrating glycans. An explanation of this illustration system is available on the internet at www.ncbi.nlm.nih.gov/glycans/snfg.html, the entire contents of which are herein incorporated by reference in its entirety for all purposes. Symbol Nomenclature for Graphical Representation of Glycans as published in Glycobiology 25: 1323-1324, 2015, which is available on the internet at doi.org/10.1093/glycob/cwv091. Alternatively, Table 7A shows a greyscale depiction of the SNFG for illustrating glycans used herein.

Within this system, the term, Hex_i: is interpreted as follows: i indicates the number of green circles (mannose) and the number of yellow circles (galactose). The term, HexNAC_j, uses j to indicate the number of blue squares (GlcNAC's). The term Fuc_d, uses d to indicate the number of red triangles (fucose). The term Neu₅AC_1, uses 1 to indicate the number of purple diamonds (sialic acid). The glycan reference codes used herein combine these i, j, d, and l terms to make a composite 4-5 number glycan reference code, e.g., 5300 or 5320. As an example, glycans 3200 and 3210 in FIG. 1 both include 3 green circles (mannose), 2 blue squares (GlcNAC's), and no purple diamonds (sialic acid) but differ in that glycan 3210 also includes 1 red triangle (fucose).

As used herein, the term “glycopeptide,” refers to a peptide having at least one glycan residue bonded thereto. In each embodiment described herein, the glycopeptide may comprise, consist essentially of, or consist of, the amino acid sequence specified by the indicated SEQ ID NO together with one or more glycans, for instance those described herein associated with that SEQ ID NO. For instance, a glycopeptide according to SEQ ID NO: 1, as used herein, can refer to a glycopeptide according to the amino acid sequence of SEQ ID NO: 1 and glycan 6513, wherein the glycan is bonded to residue 107 of SEQ ID NO: 1. Similarly usage applies to SEQ ID NOs: 2-38, with the glycans described in sections below.

As used herein, the term “glycoform” refers to a unique primary, secondary, tertiary and quaternary structure of a protein with an attached glycan of a specific structure.

As used herein, the phrase “glycosylated peptides,” refers to a peptide bonded to a glycan.

As used herein, the phrase “glycopeptide fragment” or “glycosylated peptide fragment” or “glycopeptide” refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

As used herein, the phrase “multiple reaction monitoring mass spectrometry (MRM-MS),” refers to a highly sensitive and selective method for the targeted quantification of glycans and peptides in biological samples. Unlike traditional mass spectrometry, MRM-MS is highly selective (targeted), allowing researchers to fine tune an instrument to specifically look for certain peptides fragments of interest. MRM allows for greater sensitivity, specificity, speed and quantitation of peptides fragments of interest, such as a potential biomarker. MRM-MS involves using one or more of a triple quadrupole (QQQ) mass spectrometer and a quadrupole time-of-flight (qTOF) mass spectrometer.

As used herein, the phrase “digesting a glycopeptide,” refers to a biological process that employs enzymes to break specific amino acid peptide bonds. For example, digesting a glycopeptide includes contacting a glycopeptide with an digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing.

As used herein, the phrase “fragmenting a glycopeptide,” refers to the ion fragmentation process which occurs in a MRM-MS instrument. Fragmenting may produce various fragments having the same mass but varying with respect to their charge.

As used herein, the term “subject,” refers to a mammal. The non-liming examples of a mammal include a human, non-human primate, mouse, rat, dog, cat, horse, or cow, and the like. Mammals other than humans can be advantageously used as subjects that represent animal models of disease, pre-disease, or a pre-disease condition. A subject can be male or female. However, in the context of diagnosing ovarian cancer, the subject is female unless explicitly specified otherwise. A subject can be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition, such as ovarian cancer.

As used herein, the term “patient” refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

As used herein, the phrase “multiple-reaction-monitoring (MRM) transition,” refers to the mass to charge (m/z) peaks or signals observed when a glycopeptide, or a fragment thereof, is detected by MRM-MS. The MRM transition is detected as the transition of the precursor and product ion.

As used herein, the phrase “detecting a multiple-reaction-monitoring (MRM) transition,” refers to the process in which a mass spectrometer analyzes a sample using tandem mass spectrometer ion fragmentation methods and identifies the mass to charge ratio for ion fragments in a sample. The phrase also refers to refers to a MS process in which a MRM-MS transition is detected and then compare to a calculated mass to charge ratio (m/z) of a glycopeptide, or fragment thereof, in order to identify the glycopeptide. The absolute value of these identified mass to charge ratios are referred to as transitions. In the context of the methods set forth herein, the mass to charge ratio transitions are the values indicative of glycan, peptide or glycopeptide ion fragments. For some glycopeptides set forth herein, there is a single transition peak or signal. For some other glycopeptides set forth herein, there is more than one transition peak or signal. In some examples, herein, a single transition may be indicative of two more glycopeptides, if those glycopeptides have identical MRM-MS fragmentation patterns. A transition peak or signal includes, but is not limited to, those transitions set forth herein were are associated with a glycopeptide consisting of, or consisting essentially of, an amino acid sequence selected from SEQ ID NOs: 1-38, and combinations thereof, according to Tables 1-5, e.g., Table 1, Table 2, Table 3, Table 4, or Table 5, or a combination thereof. Background information on MRM mass spectrometry can be found in Introduction to Mass Spectrometry: Instrumentation, Applications, and Strategies for Data Interpretation, 4th Edition, J. Throck Watson, O. David Sparkman, ISBN: 978-0-470-51634-8, November 2007, the entire contents of which are here incorporated by reference in its entirety for all purposes.

As used herein, the term “reference value” refers to a value obtained from a population of individual(s) whose disease state is known. The reference value may be in n-dimensional feature space and may be defined by a maximum-margin hyperplane. A reference value can be determined for any particular population, subpopulation, or group of individuals according to standard methods well known to those of skill in the art.

As used herein, the term “population of individuals” means one or more individuals. In one embodiment, the population of individuals consists of one individual. In one embodiment, the population of individuals comprises multiple individuals. As used herein, the term “multiple” means at least 2 (such as at least 4, 6, 8, 10, 12, 14, 16, 18, 20, 22, 24, 26, 28, or 30) individuals. In one embodiment, the population of individuals comprises at least 10 individuals.

As used herein, the term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms. Treating may include administering therapeutic agents to a subject in need thereof.

As used herein, the term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g., —NH₂), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refers to a biological process that employs enzymes to break specific amino acid peptide bonds. For example, digesting a peptide includes contacting the peptide with an digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein. A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio,” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” As used herein, the phrase “peptide,” is meant to include glycopeptides unless stated otherwise.

The term “peptide structure,” as used herein, generally refers to peptides or a portion thereof or glycopeptides or a portion thereof. In various embodiments described herein, a peptide structure can include any molecule comprising at least two amino acids in sequence. A peptide structure may comprise a peptide with its associated glycan.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ(H₂O)ₙ).

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### II. Overview of Exemplary Workflow

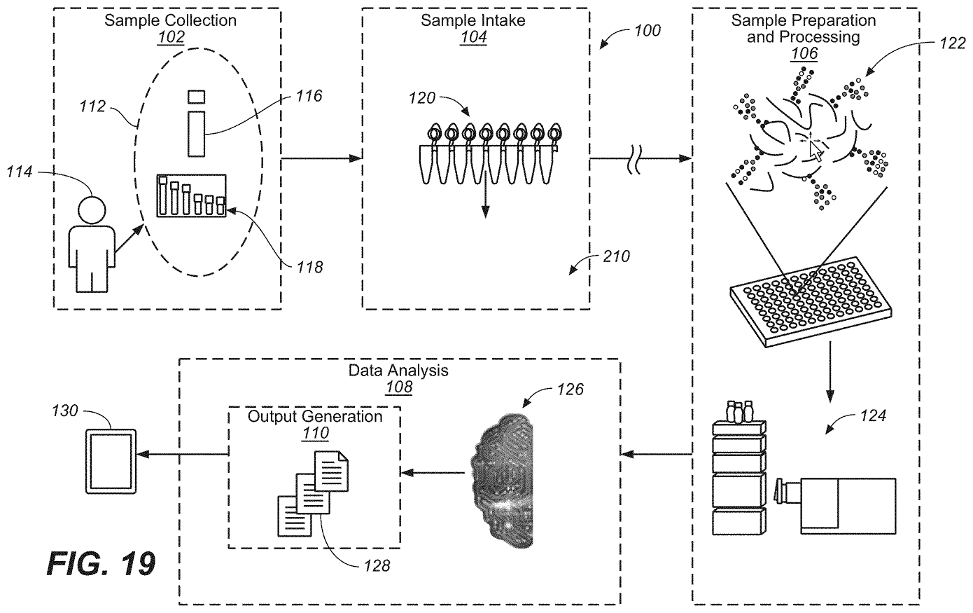

FIG. 19 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### III. Detection and Quantification of Peptide Structures

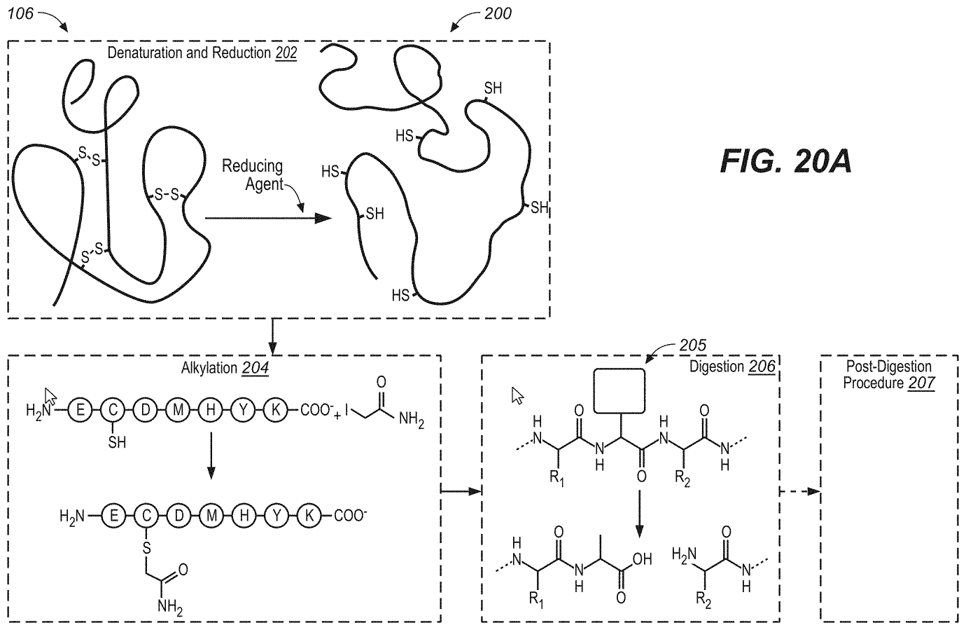

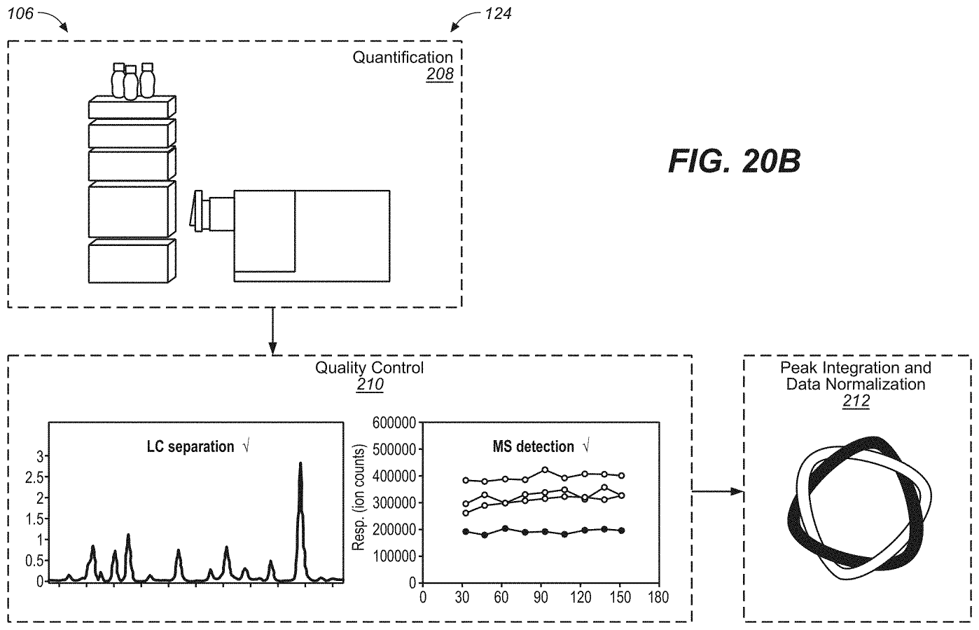

FIGS. 20A and 20B are schematic diagrams of a workflow for sample preparation and processing 106 in accordance with one or more embodiments. FIGS. 20A and 20B are described with continuing reference to FIG. 19. Sample preparation and processing 106 may include, for example, preparation workflow 200 shown in FIG. 20A and data acquisition 124 shown in FIG. 20B.

III.A. Sample Preparation and Processing

FIG. 20A is a schematic diagram of preparation workflow 200 in accordance with one or more embodiments. Preparation workflow 200 may be used to prepare a sample, such as a sample of set of samples 120 in FIG. 19, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 19). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

III.B. Peptide Structure Identification and Quantitation

FIG. 20B is a schematic diagram of data acquisition 124 in accordance with one or more embodiments. In various embodiments, data acquisition 124 can commence following sample preparation 200 described in FIG. 20A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### IV. Peptide Structure Data Analysis

IV.A. Exemplary System for Peptide Structure Data Analysis

IV.A.1. Analysis System for Peptide Structure Data Analysis

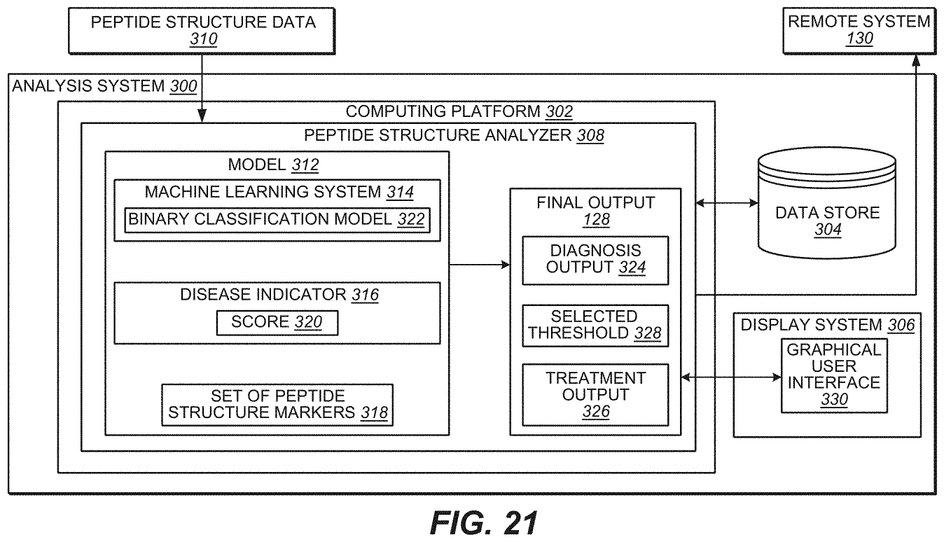

FIG. 21 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 19. Thus, analysis system 300 is described with continuing reference to workflow 100 as described in FIGS. 19, 20A, and/or 20B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 19, 20A, and 20B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for an ovarian cancer disease state based on set of peptide structures 318 identified as being associated with the ovarian cancer disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the ovarian cancer disease state. In various embodiments, disease indicator 316 can include a score 320. Score 320 indicates whether the ovarian cancer disease state is present or not. For example, score 320 may be, a probability score that indicates how likely it is that the biological sample 112 evidences the presence of the ovarian cancer disease state.

In one or more embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the ovarian cancer disease state based on training of model 312. In one or more embodiments, set of peptide structures 318 includes at least one, at least two, or at least three peptide structures from a first group of peptide structures (peptide structures PS-1 through PS-10) identified in Table 1A in Section VI.A. or at least one, at least two, or at least three peptide structures from a second group of peptide structures (peptide structures PS-5 and PS-11 through PS-34) identified in Table 2A in Section VI.A. For example, in one or more embodiments, set of peptide structures 318 includes at least 1, at least 2, at least 3, at least 4, at least 5, at least 6, at least 7, at least 8, at least 9, or all 10 of the peptide structures identified in Table 1A below in Section VI.A. In one or more other embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures identified in Table 2A below in Section VI.A. In one or more embodiments, set of peptide structures 318 includes at least peptide structure PS-5, which is identified in both Table 1A and Table 2A. In some cases, the number of peptide structures selected from Table 1A for inclusion in set of peptide structures 318 may be based on, for example, a desired level of accuracy.

In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures identified in Table 3A below in Section VI.A. In one or more embodiments, set of peptide structures 318 includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, or all 61 of the peptide structures listed in Tables 1A, 2A, and 3A.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In one or more embodiments, final output 128 includes diagnosis output 324, treatment output 326, or both. Diagnosis output 324 may include, for example, a diagnosis for the ovarian cancer disease state. The diagnosis can include a positive diagnosis or a negative diagnosis for the ovarian cancer disease state. In one or more embodiments, generating diagnosis output 324 may include comparing score 320 to selected threshold 328 to determine the diagnosis. Selected threshold 328 may be, for example, without limitation, a score between 0.30 and 0.65 (e.g., 0.4, 0.5, 0.6, etc.). For example, when selected threshold 328 is set to 0.5, a score 320 above 0.5 (or at or above 0.5) may indicate the presence of the ovarian cancer disease state and be output in diagnosis output 324 as a positive diagnosis. A score 320 below 0.5 (or at or below 0.5) may indicate that the ovarian cancer disease state is not present and be output in diagnosis output 324 as a negative diagnosis. In one or more embodiments, a negative diagnosis indicates that the subject is healthy. In one or more embodiments, a negative diagnosis indicates that a detected pelvic tumor (or mass) is benign.

In one or more embodiments, when disease indicator 316 and/or diagnosis output 324 indicate a positive diagnosis for the ovarian cancer disease state, a biopsy may be recommended. For example, a biopsy of the subject may be performed in response to disease indicator 316 and/or diagnosis output 324 indicating a positive diagnosis for the ovarian cancer disease state. In some embodiments, peptide structure analyzer 308 (or another system implemented on computing platform 302) may generate a report recommending that a biopsy is to be performed for the subject in response to disease indicator 316 and/or diagnosis output 324 indicating a positive diagnosis for the ovarian cancer disease state. In other embodiments, peptide structure analyzer 308 may send diagnosis final output 128 to remote system 130 over one or more wireless, wired, and/or optical communications links and remote system 130 may generate a report recommending that a biopsy is to be performed for the subject in response to disease indicator 316 and/or diagnosis output 324 indicating a positive diagnosis for the ovarian cancer disease state. The biopsy may be used to confirm the diagnosis to determine whether or not to administer treatment and/or how quickly to administer treatment. When disease indicator 316 and/or diagnosis output 324 indicate a negative diagnosis for the ovarian cancer disease state (e.g., benign pelvic tumor), the report that is generated by peptide structure analyzer 308, remote system 130, or some other system implemented on computing platform 142 may recommend a period of monitoring for the subject. For example, a negative diagnosis indication by disease indicator 316 and/or diagnosis output 324 may thus help prevent unnecessary treatment or overtreatment of the subject.

Treatment output 326 may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 330 in display system 306 for viewing by a human operator.

IV.A.2. Computer Implemented System

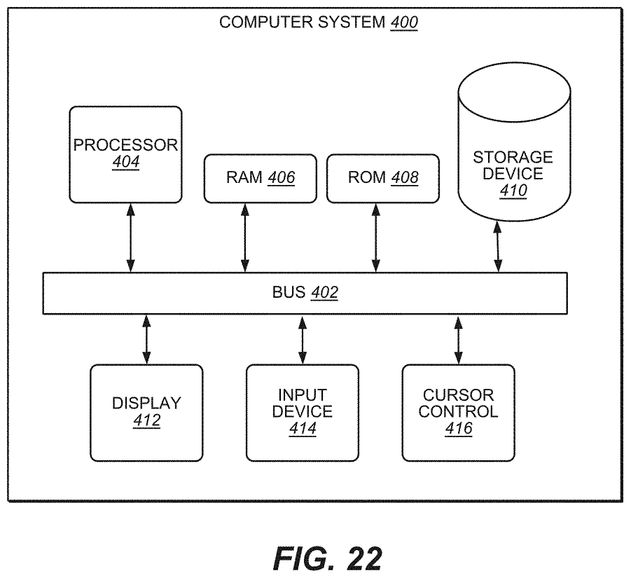

FIG. 22 is a block diagram of a computer system in accordance with various embodiments. Computer system 400 may be an example of one implementation for computing platform 302 described above in FIG. 21.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### V. Exemplary Methodologies Relating to Diagnosis Based on Peptide Structure Data Analysis

V.A. Exemplary Methodology—Based on Tables 1A and 2A

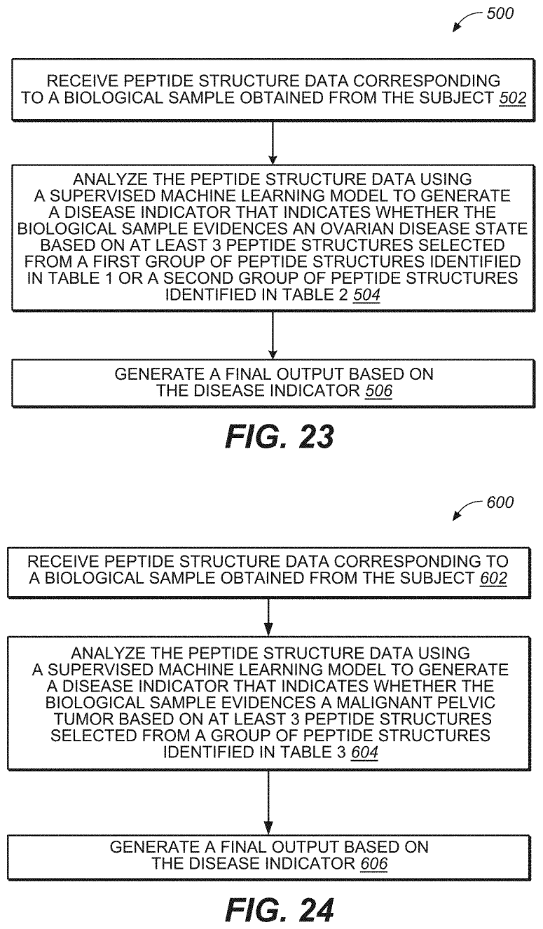

FIG. 23 is a flowchart of a process for diagnosing a subject with respect to an ovarian cancer disease state in accordance with one or more embodiments. Process 500 may be implemented using, for example, at least a portion of workflow 100 as described in FIGS. 19, 20A, and 20B and/or analysis system 300 as described in FIG. 21. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 21. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1A or Table 2A, with the peptide sequence being one of SEQ ID NOS: 111-119 in Table 1A or one of SEQ ID NOS: 114, 115, and 131-146 in Table 2A, the SEQ ID NOS being defined in Table 5A below.

Step 504 includes analyzing the peptide structure data using a supervised machine learning model to generate a disease indicator that indicates whether the biological sample evidences an ovarian cancer disease state based on at least three peptide structures selected from a first group of peptide structures identified in Table 1A (below) or a second group of peptide structures identified in Table 2A (below). In step 504, the first and second groups of peptide structures are associated with the ovarian cancer disease state. The first group of peptide structures is listed in Table 1A with respect to relative significance to the disease indicator. The second group of peptide structures is listed in Table 2A with respect to relative significance to the disease indicator.

The first group of peptide structures in Table 1A includes peptide structures that have been determined relevant to distinguishing at least between ovarian cancer (e.g., EOC) and a healthy state. For example, the first group of peptide structures may be used to predict the probability of EOC for use in clinically screening patients. In one or more embodiments, the first group of peptide structures in Table 1A may also be peptide structures that have been determined relevant to distinguishing between ovarian cancer (e.g., EOC) and a benign tumor state (e.g., a benign pelvic tumor). For example, the first group of peptide structures may be used to clinically triage patients that have been identified as having pelvic tumors to determine the probability that such a tumor evidences EOC.

The second group of peptide structures in Table 2A includes peptide structures that have been determined relevant to distinguishing at least between ovarian cancer (e.g., EOC) and the benign tumor state (e.g., a benign pelvic tumor). For example, the second group of peptide structures may be used to clinically triage patients that have been identified as having pelvic tumors to determine the probability that such a tumor evidences EOC. In this manner, the second group of peptide structures may predict malignancy of an identified pelvic tumor.

In one or more embodiments, the at least 3 peptide structures includes at least 3, at least 4, at least 5, at least 6, at least 7, at least 8, at least 9, or all 10 of the peptide structures PS-1 to PS-10 in Table 1A. In some embodiments, the at least 3 peptide structures include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-5 and PS-11 through PS-34 in Table 1A. In some embodiments, the at least 3 peptide structures includes at least PS-5, which is present in both Table 1A and Table 2A.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the ovarian cancer disease state and the supervised machine learning model is configured to generate an output that identifies the biological sample as either evidencing (“positive for”) the ovarian cancer disease state when the disease indicator is greater than a selected threshold or not evidencing (“negative for”) the ovarian cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 506 includes generating a final output based on the disease indicator. The final output may include a diagnosis output, such as, for example, diagnosis output 324 in FIG. 21. The diagnosis output may include the disease indicator, or a diagnosis made based on the disease indicator. The diagnosis may be, for example, “positive” for the ovarian cancer disease state if the biological sample evidences the ovarian cancer disease state based on the disease indicator. The diagnosis may be, for example, “negative” if the biological sample does not evidence the ovarian cancer disease state based on the disease indicator. A negative diagnosis may mean that the biological sample has a non-ovarian cancer state. The negative diagnosis for the ovarian cancer disease state can include at least one of a healthy state, a benign tumor state, or some other non-malignant state.

Generating the diagnosis output in step 506 may include determining that the score falls above (or at or above) a selected threshold and generating a positive diagnosis for the ovarian cancer disease state. Alternatively, step 506 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 506 may include a treatment output if the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 1A below lists a first group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of screening for malignant pelvic tumors). The first group of peptide structures is listed in Table 1A in order with respect to relative significance to the disease indicator. In training, testing, and predictive use of this model, the quantification metrics for peptide structure PS-9, peptide structure PS-10, or a combination of the two may form one input. Table 1A also identifies check markers CK-1 and CK-2, which may also be used by the model.

TABLE 1A
1^{st} Group of Peptide Structures Associated with Ovarian Cancer
(may be used to distinguish between malignant pelvic tumor (e.g., EOC) and healthy)

| | | | | | Linking | Linking | |
|---|---|---|---|---|---|---|---|
| | | | | Mono- | Site Pos. | Site Pos. | Glycan |
| PS- | Peptide | (Protein) | (Peptide) | isotopic | in | in | Structure |
| ID | Structure (PS) | SEQ ID | SEQ ID | mass | Protein | Peptide | GL |
| NO. | NAME | NO. | NO. | (Da) | Sequence | Sequence | NO. |
| PS-1 | ZA2G_128_5402 | 101 | 111 | 3342.26 | 128 | 8 | 5402 |
| PS-2 | IC1_253_6503 | 102 | 112 | 4961.09 | 253 | 4 | 6503 |
| PS-3 | CFAI_494_5402 | 103 | 113 | 3025.18 | 494 | 4 | 5402 |
| PS-4 | CERU_138_6513 | 104 | 114 | 4898.89 | 138 | 10 | 6513 |
| PS-5 | IGG1_297_3410 | 105 | 115 | 2633.04 | 180 | 5 | 3410 |
| PS-6 | HEMO_64_5402 | 106 | 116 | 4731.84 | 64 | 15 | 5402 |
| PS-7 | APOB_983_5402 | 107 | 117 | 5754.34 | 983 | 16 | 5402 |
| PS-8 | HPT_207_121005 | 108 | 118 | 6888.63 | 207 | 5, 9 | 121005 |
| CK-1 | FINC_ | N/A | N/A | N/A | N/A | N/A | N/A |
| | SYTITGLQPGTDYK | | | | | | |
| PS-9 | IGG3_297_3400 | 109 | 119 | 2470.99 | 227 | 5 | 3400 |
| PS-10 | IGG4_297_3400 | 110 | 120 | 2470.99 | 227 | 5 | 3400 |
| CK-2 | APOM_135_ | N/A | N/A | N/A | N/A | N/A | N/A |
| | 8500_CHK | | | | | | |

Table 2A below lists a second group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of triaging to distinguish between malignant and benign pelvic tumors). The second group of peptide structures is listed in Table 2A in order with respect to relative significance to the disease indicator. Table 2A also identifies check markers CK-3 and CK-4, which may also be used by the model.

TABLE 2A
2^{nd} Group of Peptide Structures Associated with Ovarian Cancer
(may be used to distinguish between malignant v. benign pelvic tumors)

| | | | | | Linking | Linking | |
|---|---|---|---|---|---|---|---|
| | | | | Mono- | Site Pos. | Site Pos. | Glycan |
| PS- | Peptide | (Protein) | (Peptide) | isotopic | in | in | Structure |
| ID | Structure (PS) | SEQ ID | SEQ ID | mass | Protein | Peptide | GL |
| NO. | NAME | NO. | NO. | (Da) | Sequence | Sequence | NO. |
| CK-3 | APOD_98_9800_ | N/A | N/A | N/A | N/A | N/A | N/A |
| | CHECK | | | | | | |
| PS-11 | CO2_621_5200 | 120 | 131 | 2670.19 | 621 | 11 | 5200 |
| PS-5 | IGG1_297_3410 | 105 | 115 | 2633.04 | 180 | 5 | 3410 |
| PS-12 | AGP1_93_7612 | 121 | 132 | 4995.98 | 93 | 7 | 7612 |
| PS-13 | AACT_271_7602 | 122 | 133 | 4686.91 | 271 | 4 | 7602 |
| PS-14 | A2MG_1424_ | 123 | 134 | 4366.95 | 1424 | 3 | 5402 |
| | 5402 | | | | | | |
| PS-15 | AACT_271_6513 | 122 | 133 | 4758.93 | 271 | 4 | 6513 |
| PS-16 | CERU_397_5402 | 104 | 135 | 4330.76 | 397 | 2 | 5402 |
| PS-17 | APOB_3411_ | 107 | 136 | 3316.40 | 3411 | 7 | 5301 |
| | 5301 | | | | | | |
| PS-18 | AACT_106_6513 | 122 | 137 | 5406.24 | 106 | 2 | 6513 |
| PS-19 | CERU_138_5402 | 104 | 114 | 4096.61 | 138 | 10 | 5402 |
| PS-20 | A1AT_107_6513 | 124 | 138 | 6697.87 | 107 | 14 | 6513 |
| PS-21 | AGP1_93_7602 | 121 | 132 | 4849.93 | 93 | 7 | 7602 |
| PS-22 | VTNC_242_6502 | 125 | 139 | 5341.22 | 242 | 1 | 6502 |
| PS-23 | IGG2_297_3510 | 126 | 140 | 2804.13 | 176 | 5 | 3510 |
| PS-24 | CFAH_882_5411 | 127 | 141 | 4079.71 | 882 | 15 | 5411 |
| CK-4 | APOM_135_ | N/A | N/A | N/A | N/A | N/A | N/A |
| | 8500_CHECK | | | | | | |
| PS-25 | AGP1_103_8704 | 121 | 142 | 4657.74 | 103 | 2 | 8704 |
| PS-26 | IGG1_297_4300 | 105 | 115 | 2445.95 | 180 | 5 | 4300 |
| PS-27 | APOH_253_5401 | 128 | 143 | 3163.24 | 253 | 3 | 5401 |
| PS-28 | APOD_98_5411 | 129 | 144 | 4312.85 | 98 | 16 | 5411 |
| PS-29 | TRFE_630_5411 | 130 | 145 | 4573.85 | 630 | 9 | 5411 |
| PS-30 | CERU_138_6502 | 104 | 114 | 4461.74 | 138 | 10 | 6502 |
| PS-31 | A2MG_1424_ | 123 | 134 | 4221.91 | 1424 | 3 | 5411 |
| | 5411 | | | | | | |
| PS-32 | A2MG_55_5411 | 123 | 146 | 4455.96 | 55 | 9 | 5411 |
| PS-33 | TRFE_630_5412 | 130 | 145 | 4864.95 | 630 | 9 | 5412 |
| PS-34 | IGG2_297_4511 | 126 | 140 | 3257.28 | 176 | 5 | 4511 |

V.B. Exemplary Methodology—Based on Table 3A

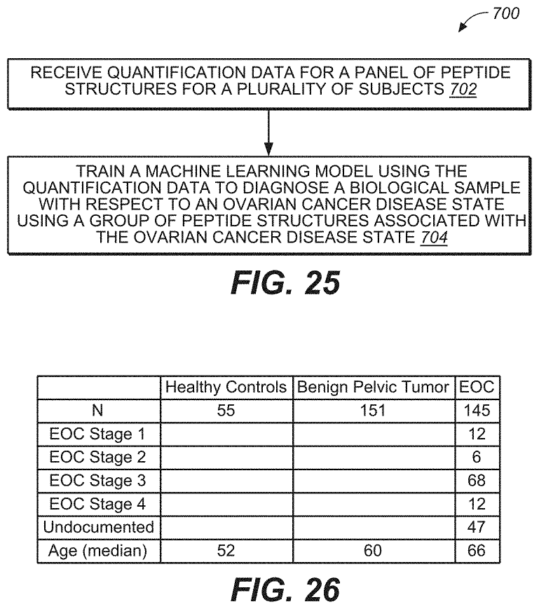

FIG. 24 is a flowchart of a process for diagnosing a subject with respect to an ovarian cancer disease state in accordance with one or more embodiments. Process 600 may be implemented using, for example, at least a portion of workflow 100 as described in FIGS. 19, 20A, and 20B and/or analysis system 300 as described in FIG. 21. Process 600 may be used to generate a final output that includes at least a diagnosis output for the subject.

Step 602 includes receiving peptide structure data corresponding to a biological sample obtained from the subject. The peptide structure data may be, for example, one example of an implementation of peptide structure data 310 in FIG. 21. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 3A, with the peptide sequence being one of SEQ ID NOS: 111, 114, 115, 131, 132, 133, 134, 137, 138, 140, 142, 144, 145, 146, 153-165 in Table 3A, the SEQ ID NOS being defined in Table 5A below.

Step 604 includes analyzing the peptide structure data using a supervised machine learning model to generate a disease indicator that predicts whether the biological sample evidences a malignant pelvic tumor or benign pelvic tumor based on at least three peptide structures selected from a group of peptide structures identified in Table 3A. The group of peptide structures is listed in Table 3A with respect to relative significance to the disease indicator, which may be a probability score. In step 604, the group of peptide structures is associated with the malignancy (e.g., EOC). For example, the group of peptide structures in Table 3A includes peptide structures that have been determined relevant to distinguishing between a malignant and benign nature of a pelvic tumor.

In one or more embodiments, the at least 3 peptide structures includes at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-1, PS-5, PS-11, PS-15, PS-20, PS-25, PS-28, PS-29, PS-30, PS-31, PS-32, and PS-35 to PS-61 identified in Table 3A.

In one or more embodiments, step 604 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 604 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is evidences malignancy (e.g., EOC) and the supervised machine learning model is configured to generate an output that identifies the biological sample as either evidencing (“positive for”) malignancy when the disease indicator is greater than a selected threshold or not evidencing (“negative for”) malignancy when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 606 includes generating a final output based on the disease indicator. The final output may include a diagnosis output, such as, for example, diagnosis output 324 in FIG. 21. The diagnosis output may include the disease indicator, or a diagnosis made based on the disease indicator. The diagnosis may be, for example, “positive” for an ovarian cancer disease state (e.g., EOC) if the biological sample evidences malignancy based on the disease indicator. The diagnosis may be, for example, “negative” if the biological sample does not evidence malignancy based on the disease indicator. A negative diagnosis may mean that the biological sample evidences a benign status (or a non-ovarian cancer state).

Generating the diagnosis output in step 606 may include determining that the score falls above (or at or above) a selected threshold and generating a positive diagnosis for the ovarian cancer disease state. Alternatively, step 606 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 606 may include a treatment output if the disease indicator predicts malignancy and/or the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

TABLE 3A
3^{rd} Group of Peptide Structures Associated with
Ovarian Cancer (may be used to distinguish
between malignant and benign pelvic tumors)

| | | | | Linking | |
|---|---|---|---|---|---|
| | | | | Site Pos. | Glycan |
| | | (Protein) | (Peptide) | in | Structure |
| PS-ID | Peptide Structure | SEQ ID | SEQ ID | Protein | GL |
| NO. | (PS) NAME | NO. | NO. | Sequence | NO. |
| PS-35 | VTNC_169_5401 | 125 | 153 | 169 | 5401 |
| PS-36 | FETUA_176_6513 | 147 | 154 | 176 | 6513 |
| PS-37 | AGP1_93_7614 | 121 | 132 | 93 | 7614 |
| PS-38 | QUANTPEP. | 148 | 155 | N/A | N/A |
| | A2GL_ | | | | |
| | DLLLPQPDLR | | | | |
| PS-39 | HPT_184_5402 | 108 | 156 | 184 | 5402 |
| PS-40 | TRFE_432_6503 | 130 | 157 | 432 | 6503 |
| PS-41 | TRFE_630_6513 | 130 | 145 | 630 | 6513 |
| PS-42 | HEMO_453_5402 | 106 | 158 | 453 | 5402 |
| PS-43 | QUANTPEP.TTR_ | 149 | 159 | N/A | N/A |
| | TSESGELHGL_ | | | | |
| | TTEEEFVEGIYK | | | | |
| PS-5 | IGG1_297_3410 | 105 | 115 | 297 | 3410 |
| PS-44 | TRFE_630_5400 | 130 | 145 | 630 | 5400 |
| PS-45 | AGP1_103_9804 | 121 | 142 | 103 | 9804 |
| PS-46 | TRFE_432_6501 | 130 | 157 | 432 | 6501 |
| PS-47 | HPT_241_5402 | 108 | 160 | 241 | 5402 |
| PS-48 | IGG1_297_5510 | 105 | 115 | 297 | 5510 |
| PS-49 | QUANTPEP. | 150 | 161 | N/A | N/A |
| | AFAM_ | | | | |
| | SDVGFLPPF_ | | | | |
| | PTLDPEEK | | | | |
| PS-32 | A2MG_55_5411 | 123 | 146 | 55 | 5411 |
| PS-50 | IGG2_297_5510 | 126 | 140 | 297 | 5510 |
| PS-51 | AGP1_103_7603 | 121 | 142 | 103 | 7603 |
| PS-52 | IGG2_297_5400 | 126 | 140 | 297 | 5400 |
| PS-1 | ZA2G_128_5402 | 101 | 111 | 128 | 5402 |
| PS-53 | TRFE_630_6502 | 130 | 145 | 630 | 6502 |
| PS-54 | TRFE_432_6502 | 130 | 157 | 432 | 6502 |
| PS-55 | IGG2_297_4510 | 126 | 140 | 297 | 4510 |
| PS-56 | AACT_106_7614 | 122 | 137 | 106 | 7614 |
| PS-57 | PEP-APOA1_ | 151 | 162 | N/A | N/A |
| | VSFLSALEEYTK | | | | |
| PS-11 | CO2_621_5200 | 120 | 131 | 621 | 5200 |
| PS-15 | AACT_271_6513 | 122 | 133 | 271 | 6513 |
| PS-58 | FETUA_176_5401 | 147 | 154 | 176 | 5401 |
| PS-59 | FETUA_346_1102 | 147 | 163 | 346 | 1102 |
| PS-60 | PEP-APOA1_ | 151 | 164 | N/A | N/A |
| | THLAPYSDELR | | | | |
| PS-29 | TRFE_630_5411 | 130 | 145 | 630 | 5411 |
| PS-25 | AGP1_103_8704 | 121 | 142 | 103 | 8704 |
| PS-30 | CERU_138_6502 | 104 | 114 | 138 | 6502 |
| PS-20 | A1AT_107_6513 | 124 | 138 | 107 | 6513 |
| PS-31 | A2MG_1424_5411 | 123 | 134 | 1424 | 5411 |
| PS-28 | APOD_98_5411 | 129 | 144 | 98 | 5411 |
| PS-61 | C4BPA_221_5402 | 152 | 165 | 221 | 5402 |

V.C. Training a Model to Predict Ovarian Cancer (e.g., Epithelial Ovarian Cancer)

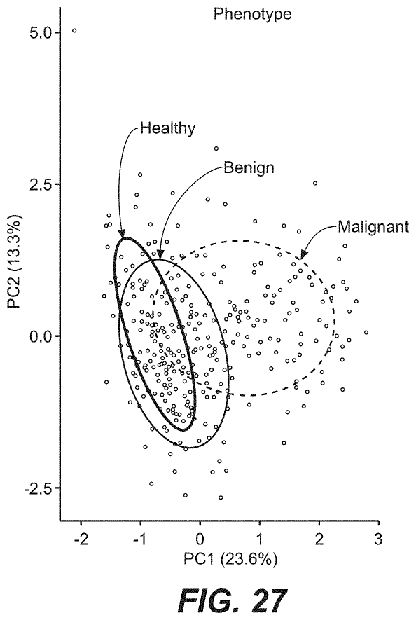

FIG. 25 is a flowchart of a process for training a model to diagnose a subject with respect to an ovarian cancer disease state in accordance with one or more embodiments. Process 700 may be implemented using, for example, at least a portion of workflow 100 as described in FIGS. 19, 20A, and 20B and/or analysis system 300 as described in FIG. 21. In some embodiments, process 700 may be one example of an implementation for training the model used in the process 500 in FIG. 23.

Step 702 includes receiving quantification data for a panel of peptide structures for a plurality of subjects. The plurality of subjects includes a first portion diagnosed with a negative diagnosis of an ovarian cancer disease state and a second portion diagnosed with a positive diagnosis of the ovarian cancer disease state. The quantification data comprises an initial plurality of peptide structure profiles for the plurality of subjects. For example, a peptide structure profile in the initial plurality of peptide structure profiles may include a feature associated with a corresponding peptide structure. The feature may be relative abundance, concentration, site occupancy, or some other quantification-based feature. The initial plurality of peptide structure profiles may include, one, two, three, or more profiles for a given peptide structure.

Step 704 includes training a machine learning model using the quantification data to diagnose a biological sample with respect to the ovarian cancer disease state using a group of peptide structures associated with the ovarian cancer disease state (e.g., the first group of peptide structures is identified in Table 1A, the second group of peptide structures is identified in Table 2A, the third group of peptide structures is identified in Table 3A). The first, second, and third groups of peptide structures are listed in Tables 1A, 2A, and 3A, respectively, with respect to relative significance to diagnosing the biological sample as evidencing malignancy (e.g., EOC). Step 704 can include training the machine learning using a portion of the quantification data corresponding to a training group of peptide structures included in the plurality of peptide structures.

Step 704 may include reducing the plurality of peptide structure profiles using LASSO regression to identify a final group of peptide structures identified in Table 1A above. Step 704 may include reducing the plurality of peptide structure profiles using LASSO regression to identify a final group of peptide structures identified in Table 2A above.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the ovarian cancer disease state and a negative diagnosis for any subject of the plurality of subjects determined not to have the ovarian cancer disease state.

The machine learning model can include a binary classification model. Some binary classification models can include logistical regression models. Some logistical regression models can include LASSO regression models.

An alternative or additional step in process 700 can include filtering the initial plurality of peptide structure profiles by a coefficient of variation to generate a plurality of peptide structure profiles for use in training the machine learning model. As one example, only those peptide structure profiles having a low coefficient of variation (<20%) were included int the plurality of peptide structure profiles used for training.

An alternative or additional step in process 700 can include performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state.

An alternative or additional step in process 700 can include identifying a first portion of the plurality of samples for subjects with benign pelvic tumors and malignant pelvic tumors and a second portion of the plurality of samples for subjects with a healthy status. An alternative or additional step in process 700 can include generating a training set of peptide structure profiles for 80% of the first portion and a test set of peptide structure profiles for a remaining 20% of the first portion and the second portion.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

V.D. Is Methods of Treating Ovarian Cancer

In one or more embodiments, the final output generated in step 506 in FIG. 23 or in step 606 in FIG. 24 may include a treatment output. The treatment output may identify one or more treatment types for a subject based on the disease indicator and/or diagnosis output generated via process 500 in FIG. 23 or process 600 in FIG. 24, respectively. Treatment for ovarian cancer (e.g., EOC) may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment output may include, for example, a treatment plan. The treatment plan may include, for example, but is not limited to, a timeline or schedule for administering the treatment, dosing information, other treatment-related information, or a combination thereof. Being able to accurately predict malignancy via the process 500 in FIG. 23 and/or the process 600 in FIG. 24 may allow treatment for malignant pelvic tumors (e.g., EOC) to be started earlier without requiring, in many or most cases, further invasive testing such as a biopsy.

In one or more embodiments, a patient biological sample is obtained from a subject. The biological sample may be processed (e.g., via digestion and fragmentation) such that one or more peptide structures of interest are detected. For example, detection and quantification may be performed for one or more peptide structures from Table 1A, Table 2A, and/or Table 3A. The quantification data that is generated for these peptide structures may be input into a trained binary classification model to generate a disease indicator, which may be, for example, a probability score. A determination may be made as to whether the disease indicator (e.g., score) is above or below a selected threshold (e.g., 0.5). If the disease indicator is above the selected threshold, the biological sample may be classified as evidencing malignant pelvic tumor.

Further, this classification may further include a classification that the subject is in need of treatment. If the subject is in need of treatment based on the classification, treatment is administered. For example, a therapeutically effective amount of a therapeutic agent is administered to the patient, where the therapeutic agent is selected from a chemotherapeutic agent, an immunotherapeutic agent, a hormone therapy, a targeted therapeutic agent, a neoadjuvant therapy, or a combination.

In some embodiments, provided herein is a method of treating ovarian cancer in a subject based upon the presence, absence, or amounts of one or more peptide structure provided herein (such as those in Table 1A, Table 2A, or Table 3A. In some embodiments, the method comprises detecting one or more glycopeptide herein, and treating the patient for ovarian cancer based upon the presence, absence, or amount of a glycopeptide structure defined by a peptide sequence and a glycan structure linked to the peptide sequence at a linking site of the peptide sequence, as identified in Table 3A, with the peptide sequence being one of SEQ ID NOS: 111, 114, 115, 131, 132, 133, 134, 137, 138, 140, 142, 144, 145, 146, 153-165.

### VI. Peptide Structure and Product Ion Compositions, Kits and Reagents

Aspects of the disclosure include compositions comprising one or more of the peptide structures listed in Table 1A, in Table 2A, or in Table 3A. In some embodiments, a composition comprises a plurality of the peptide structures listed in Table 1A, a plurality of the peptide structures listed in Table 2A, or a plurality of the peptide structures listed in Table 3A. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, or all 61 of the peptide structures listed in Tables 1A, 2A, and 3A. In one or more embodiments, a composition comprises 1, 2, 3, 4, 5, 6, 7, 8, 9, or all 10 of the peptide structures listed in Table 1A. In one or more embodiments, a composition comprises 1, 2, 3, 4, 5, 6, 7, 8, 9, 10, 11, 12, 13, 14, 15, 16, 17, 18, 19, 20, 21, 22, 23, 24, or all 25 of the peptide structures listed in Table 2A. In one or more embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or all 38 of the peptide structures listed in Table 3A.

In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 111-119, 131-146, and 153-165 listed in Tables 1A, 2A and 3A.

Aspects of the disclosure include compositions comprising one or more precursor ions having a defined charge and/or defined mass-to-charge (m/z) ratio, as listed in Table 4A. Aspects of the disclosure include compositions comprising one or more product ions having a defined mass-to-charge (m/z) ratio, which product ions are produced by converting a peptide structure described herein (e.g., a peptide structure listed in Tables 1A, 2A, or 3A) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Tables 1A, 2A, or 3A). In some embodiments, a composition comprises a set of the product ions listed in Table 4A, having an m/z ratio selected from the list provided for each peptide structure in Table 4A.

In some embodiments, a composition comprises at least one of peptide structures PS-1 to PS-10 identified in Table 1A. In some embodiments, a composition comprises at least one of peptide structures PS-11 to PS-34 and PS-5 identified in Table 2A. In some embodiments, a composition comprises at least one of peptide structures PS-1, PS-5, PS-11, PS-15, PS-20, PS-25, PS-28, PS-29, PS-30, PS-31, PS-32, and PS-35 to PS-61 identified in Table 3A.

In one or more embodiments, a composition comprises at least 1, at least 2, at least 3, at least 4, at least 5, at least 6, at least 7, at least 8, at least 9, or all 10 of the peptide structures PS-1 to PS-10 identified in Table 1A. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-11 to PS-34 and PS-5 identified in Table 2A. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-1, PS-5, PS-11, PS-15, PS-20, PS-25, PS-28, PS-29, PS-30, PS-31, PS-32, and PS-35 to PS-61 identified in Table 3A. In some embodiments, the at least 3 peptide structures additionally include at least 1, at least 2, at least 3, at least 4, at least 5, at least 6, or all 7 of the remaining peptide structures PS-1, PS-5, PS-11, PS-15, PS-20, PS-25, PS-28, PS-29, PS-30, PS-31, PS-32, and PS-35 to PS-61 identified in Table 3A.

In some embodiments, a composition comprises a peptide structure or a product ion. The peptide structure or product ion can include an amino acid sequence having at least 90% sequence identity to any one of SEQ ID NOS: 111-119, as identified in Table 5A, corresponding to peptide structures PS-1 to PS-10 in Table 1A. The peptide structure or product ion can include an amino acid sequence having at least 90% sequence identity to any one of SEQ ID NOS: 114, 115, 131-146, as identified in Table 5A, corresponding to various ones of peptide structures PS-5 and PS-11 to PS-34 in Table 2A. The peptide structure or product ion can include an amino acid sequence having at least 90% sequence identity to any one of SEQ ID NOS: 111, 114, 115, 131, 132, 133, 134, 137, 138, 140, 142, 144, 145, 146, 153-165, as identified in Table 5A, corresponding to various ones of peptide structures PS-1, PS-5, PS-11, PS-15, PS-20, PS-25, PS-28, PS-29, PS-30, PS-31, PS-32, and PS-35 to PS-61 in Table 3A.

In some embodiments, the product ion is selected as one from a group consisting of product ions identified in Table 4A, including product ions falling within an identified m/z range of the m/z ratio identified in Table 4A and characterized as having a precursor ion having an m/z ratio within an identified m/z range of the m/z ratio identified in Table 4A. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 4A, and characterized as having a precursor ion having an m/z ratio that falls within at least one of first range (±0.5), a second range (±1.0), or a third range (±1.0 of the precursor ion m/z ratio identified in Table 4A.

TABLE 4A
Mass Spectrometry-Related Characteristics for the Peptide
Structures associated with Ovarian Cancer (e.g., EOC)

| PS- | | Col- | Pre- | Pre- | Pro- | 2^{nd} | 2^{nd} |
|---|---|---|---|---|---|---|---|
| ID | RT | lision | cursor | cursor | duct | Collision | Product |
| NO. | (min) | Energy | m/z | Charge | m/z | Energy | m/z |
| PS-1 | 10.6 | 30 | 1115.1 | 3 | 366.1 | 34 | 1341.6 |
| PS-2 | 35.8 | 35 | 1241.8 | 4 | 204.1 | 20 | 1152.6 |
| PS-3 | 6.6 | 25 | 1009.4 | 3 | 366.1 | N/A | N/A |
| PS-4 | 17.1 | 30 | 1226.2 | 4 | 366.1 | 30 | 1048.5 |
| PS-5 | 7.9 | 21 | 879 | 3 | 204.1 | 27 | 1392.6 |
| PS-6 | 40.5 | 35 | 1184.5 | 4 | 204.1 | N/A | N/A |
| PS-7 | 33.6 | 30 | 1440.3 | 4 | 366.1 | N/A | N/A |
| PS-8 | 13.3 | 35 | 1378.9 | 5 | 366.1 | N/A | N/A |
| PS-9 | 10.1 | 35 | 1237 | 2 | 204.1 | 20 | 1376.6 |
| PS-10 | 10.1 | 35 | 1237 | 2 | 204.1 | 20 | 1376.6 |
| PS-11 | 16.3 | 20 | 891.1 | 3 | 829.4 | 20 | 366.1 |
| PS-12 | 22.6 | 31 | 1250.3 | 4 | 366.1 | N/A | N/A |
| PS-13 | 30.2 | 28 | 1173.2 | 4 | 366.1 | 978.5 | 25 |
| PS-14 | 44 | 15 | 874.4 | 5 | 366.1 | 1183.6 | 20 |
| PS-15 | 31.3 | 30 | 1191.2 | 4 | 366.1 | 978.5 | 20 |
| PS-16 | 27.4 | 35 | 1084.2 | 4 | 204.1 | N/A | N/A |
| PS-17 | 12.6 | 28 | 1106.8 | 3 | 366.1 | N/A | N/A |
| PS-18 | 37.8 | 30 | 1082.6 | 5 | 274.1 | N/A | N/A |
| PS-19 | 16.7 | 20 | 1025.7 | 4 | 274.1 | 1048.5 | 25 |
| PS-20 | 43.3 | 34 | 1341 | 5 | 366.1 | 1299 | 34 |
| PS-21 | 22.6 | 30 | 1213.8 | 4 | 366.1 | N/A | N/A |
| PS-22 | 37.3 | 30 | 1336.3 | 4 | 366.1 | N/A | N/A |
| PS-23 | 13.1 | 13 | 935.8 | 3 | 204.1 | 1360.6 | 30 |
| PS-24 | 14.8 | 25 | 1021.4 | 4 | 366.1 | N/A | N/A |
| PS-25 | 5.7 | 29 | 1165.6 | 4 | 366.1 | 979.5 | 29 |
| PS-26 | 7.9 | 30 | 1224.5 | 2 | 366.1 | N/A | N/A |
| PS-27 | 18.5 | 33 | 1055.8 | 3 | 366.1 | 1453.6 | 35 |
| PS-28 | 23.5 | 20 | 1079.7 | 4 | 366.1 | N/A | N/A |
| PS-29 | 31 | 30 | 1144.9 | 4 | 366.1 | 1359.6 | 35 |
| PS-30 | 16.5 | 34 | 1117.2 | 4 | 366.1 | N/A | N/A |
| PS-31 | 43.5 | 22 | 1057 | 4 | 366.1 | 1184.1 | 28 |
| PS-32 | 41.5 | 22 | 1115.4 | 4 | 366.1 | 366.1 | 25 |
| PS-33 | 32.3 | 30 | 1217.7 | 4 | 366.1 | 1359.6 | 35 |
| PS-34 | 13.6 | 35 | 1087.1 | 3 | 204.1 | N/A | N/A |
| PS-35 | 24.3 | 23 | 942.4 | N/A | 366.1 | N/A | N/A |
| PS-36 | 31.1 | 34 | 1343.8 | N/A | 366.1 | N/A | N/A |
| PS-37 | 23.9 | 25 | 1116.9 | N/A | 366.1 | N/A | N/A |
| PS-38 | 31.3 | 15 | 590.3 | N/A | 725.4 | N/A | N/A |
| PS-39 | 34.2 | 25 | 1149.3 | N/A | 366.1 | N/A | N/A |
| PS-40 | 28 | 27 | 1085.4 | N/A | 366.1 | N/A | N/A |
| PS-41 | 33.8 | 27 | 1105.6 | N/A | 366.1 | N/A | N/A |
| PS-42 | 31.2 | 30 | 1314.9 | N/A | 366.1 | N/A | N/A |
| PS-43 | 34.4 | 25 | 819.1 | N/A | 855.5 | N/A | N/A |
| PS-44 | 31 | 25 | 1035.6 | N/A | 366.1 | N/A | N/A |
| PS-45 | 5.6 | 25 | 1256.8 | N/A | 366.1 | N/A | N/A |
| PS-46 | 26.4 | 20 | 1252.5 | N/A | 366.1 | N/A | N/A |
| PS-47 | 31 | 33 | 1335.3 | N/A | 366.1 | N/A | N/A |
| PS-48 | 8.1 | 20 | 1054.7 | N/A | 366.1 | N/A | N/A |
| PS-49 | 40.3 | 29 | 944.5 | N/A | 1269.6 | N/A | N/A |
| PS-50 | 13.1 | 25 | 1043.8 | N/A | 366.1 | N/A | N/A |
| PS-51 | 5.8 | 34 | 1335 | N/A | 366.1 | N/A | N/A |
| PS-52 | 13.2 | 25 | 927.7 | N/A | 366.1 | N/A | N/A |
| PS-53 | 33 | 25 | 1018.1 | N/A | 366.1 | N/A | N/A |
| PS-54 | 27.4 | 25 | 1012.7 | N/A | 366.1 | N/A | N/A |
| PS-55 | 13.2 | 15 | 989.9 | N/A | 204.1 | N/A | N/A |
| PS-56 | 38.6 | 35 | 1214.1 | N/A | 274.1 | N/A | N/A |
| PS-57 | 40 | 20 | 693.9 | N/A | 675.4 | N/A | N/A |
| PS-58 | 30.4 | 26 | 1070.4 | N/A | 366.1 | N/A | N/A |
| PS-59 | 23 | 20 | 988.8 | N/A | 274.1 | N/A | N/A |
| PS-60 | 15.7 | 12 | 453.2 | N/A | 532.2 | N/A | N/A |
| PS-61 | 37.5 | 25 | 1116.9 | N/A | 366.1 | N/A | N/A |

Table 5A defines the peptide sequences for SEQ ID NOS: 111-119, 131-146, and 153-165 from Tables 1A, 2A, and 3A, respectively. Table 5A further identifies a corresponding protein SEQ ID NO. for each peptide sequence.

TABLE 5A
Peptide SEQ ID NOS

| SEQ | | Corresponding |
|---|---|---|
| ID | | Protein |
| NO: | Peptide Sequence | SEQ ID NO: |
| 111 | FGCEIENNR | 101 |
| 112 | VLSNNSDANLELINTWVAK | 102 |
| 113 | LISNCSK | 103 |
| 114 | EHEGAIYPDNTTDFQR | 104 |
| 115 | EEQYNSTYR | 105 |
| 116 | CSDGWSFDATTLDDNGTMLFFK | 106 |
| 117 | QVFPGLNYCTSGAYSNASSTDSASYYPLTGDTR | 107 |
| 118 | NLFLNHSENATAK | 108 |
| 119 | EEQYNSTFR | 109, 110 |
| 131 | QSVPAHFVALNGSK | 120 |
| 132 | QDQCIYNTTYLNVQR | 121 |
| 133 | YTGNASALFILPDQDK | 122 |
| 134 | VSNQTLSLFFTVLQDVPVR | 123 |
| 135 | ENLTAPGSDSAVFFEQGTTR | 104 |
| 136 | FVEGSHNSTVSLTTK | 107 |
| 137 | FNLTETSEAEIHQSFQHLLR | 122 |
| 138 | ADTHDEILEGLNFNLTEIPEAQIHEGFQELLR | 124 |
| 139 | NISDGFDGIPDNVDAALALPAHSYSGR | 125 |
| 140 | EEQFNSTFR | 126 |
| 141 | IPCSQPPQIEHGTINSSR | 127 |
| 142 | ENGTISR | 121 |
| 143 | LGNWSAMPSCK | 128 |
| 144 | ADGTVNQIEGEATPVNLTEPAK | 129 |
| 145 | QQQHLFGSNVTDCSGNFCLFR | 130 |
| 146 | GCVLLSYLNETVTVSASLESVR | 123 |
| 153 | NGSLFAFR | 125 |
| 154 | AALAAFNAQNNGSNFQLEEISR | 147 |
| 155 | DLLLPQPDLR | 148 |
| 156 | MVSHHNLTTGATLINEQWLLTTAK | 108 |
| 157 | CGLVPVLAENYNK | 130 |
| 158 | ALPQPQNVTSLLGCTH | 106 |
| 159 | TSESGELHGLTTEEEFVEGIYK | 149 |
| 160 | VVLHPNYSQVDIGLIK | 108 |
| 161 | SDVGFLPPFPTLDPEEK | 150 |
| 162 | VSFLSALEEYTK | 151 |
| 163 | TVVQPSVGAAAGPVVPPCPGR | 147 |
| 164 | THLAPYSDELR | 151 |
| 165 | FSLLGHASISCTVENETIGVWRPSPPTCEK | 152 |

Table 6A identifies the proteins of SEQ ID NOS: 101-110, 120-130, and 147-152 from Tables 1A, 2A, and 3A, respectively. Table 6A identifies a corresponding protein abbreviation and protein name for each of protein SEQ ID NOS: 101-110, 120-130, and 147-152. Further, Table 6A identifies a corresponding Uniprot ID for each of protein SEQ ID NOS: 101-110, 120-130, and 147-152.

TABLE 6A
Protein SEQ ID NOS

| SEQ | | | |
|---|---|---|---|
| ID | Protein | | Uniprot |
| NO. | Abbreviation | Protein Name | ID |
| 101 | ZA2G | Zinc-alpha-2-glycoprotein | P25311 |
| 102 | IC1 | Plasma protease C1 inhibitor | P05155 |
| 103 | CFAI | Complement Factor I | P05156 |
| 104 | CERU | Ceruloplasmin | P00450 |
| 105 | IGG1 | Immunoglobulin heavy constant | P01857 |
| | | gamma 1 | |
| 106 | HEMO | Hemopexin | P02790 |
| 107 | APOB | Apolipoprotein B-100 | P04114 |
| 108 | HPT | Haptoglobin | P00738 |
| 109 | IGG3 | Immunoglobulin heavy constant | P01860 |
| | | gamma 3 | |
| 110 | IGG4 | Immunoglobulin heavy constant | P01861 |
| | | gamma 3 | |
| 120 | CO2 | ComplementC2 | P06681 |
| 121 | AGP1 | Alpha-1-acid glycoprotein 1 | P02763 |
| 122 | AACT | Alpha-1-antichymotrypsin | P01011 |
| 123 | A2MG | Alpha-2-macroglobulin | P01023 |
| 124 | A1AT | Alpha-1-antitrypsin | P01009 |
| 125 | VTNC | Vitronectin | P04004 |
| 126 | IGG2 | Immunoglobulin heavy constant | P01859 |
| | | gamma 2 | |
| 127 | CFAH | Complement Factor H | P08603 |
| 128 | APOH | Beta-2-glycoprotein1 | P02749 |
| 129 | APOD | Apolipoprotein D | P05090 |
| 130 | TRFE | Serotransferrin | P02787 |
| 147 | FETUA | Alpha-2-HS-glycoprotein | P02765 |
| 148 | A2GL | Leucine-rich Alpha-2-glycoprotein | P02750 |
| 149 | TTR | Transthyretin | P02766 |
| 150 | AFAM | Afamin | P43652 |
| 151 | APOA1 | Apolipoprotein A-I | P02647 |
| 152 | C4BPA | C4 b-binding protein alpha chain | P04003 |

Table 7A identifies and defines the glycan structures included in Tables 1A, 2A, and 3A. Table 7A identifies a coded representation of the composition for each glycan structure included in Tables 1A, 2A, and 3A. As used herein, the 4-digit GL NO. is a designation that represents the number of hexoses, the number of HexNAcs, the number of Fucoses, and the number of Neuraminic Acids.

TABLE 7A
Glycan Structure GL NOS: Composition

| Glycan | | |
|---|---|---|
| Structure | | |
| GL NO. | Structure | Composition |
| 1102 | 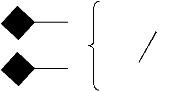 | Hex(1)HexNAc(1)Fuc(0)NeuAc(2) |
| 3400 | 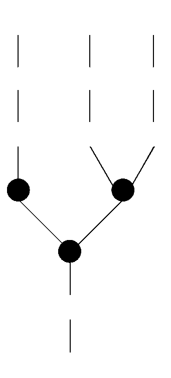 | Hex(3)HexNAc(4)Fuc(0)NeuAc(0) |
| 3410 | 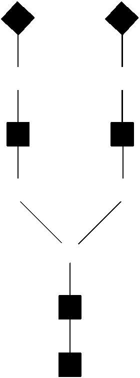 | Hex(3)HexNAc(4)Fuc(1)NeuAc(0) |
| 3510 | [symbol] | Hex(3)HexNAc(5)Fuc(1)NeuAc(0) |
| 4300 | [symbol] | Hex(4)HexNAc(3)Fuc(0)NeuAc(0) |
| 4510 | [symbol] | Hex(4)HexNAc(5)Fuc(1)NeuAc(0) |
| 4511 | 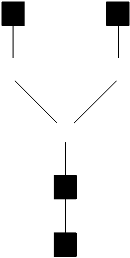 | Hex(4)HexNAc(5)Fuc(1)NeuAc(1) |
| 5200 | 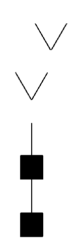 | Hex(5)HexNAc(2)Fuc(0)NeuAc(0) |
| 5301 | 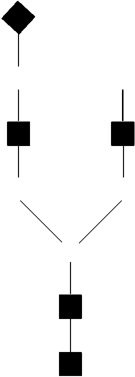 | Hex(5)HexNAc(3)Fuc(0)NeuAc(1) |
| 5400 | 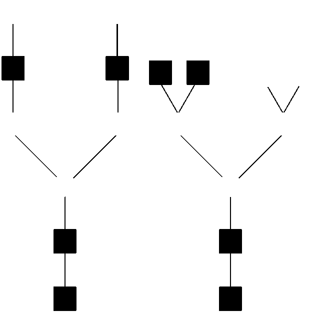 | Hex(5)HexNAc(4)Fuc(0)NeuAc(0) |
| 5401 | [symbol] | Hex(5)HexNAc(4)Fuc(0)NeuAc(1) |
| 5402 | 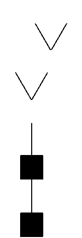 | Hex(5)HexNAc(4)Fuc(0)NeuAc(2) |
| 5411 | 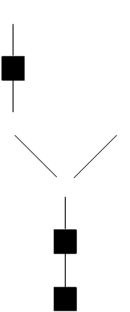 | Hex(5)HexNAc(4)Fuc(1)NeuAc(1) |
| 5412 | [symbol] | Hex(5)HexNAc(4)Fuc(1)NeuAc(2) |
| 5421 | [symbol] | Hex(5)HexNAc(4)Fuc(2)NeuAc(1) |
| 5510 | [symbol] | Hex(5)HexNAc(5)Fuc(1)NeuAc(0) |
| 6501 | 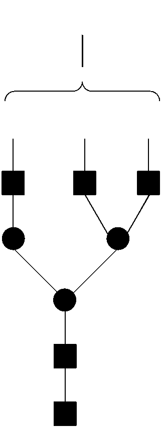 | Hex(6)HexNAc(5)Fuc(0)NeuAc(1) |
| 6502 | [symbol] | Hex(6)HexNAc(5)Fuc(0)NeuAc(2) |
| 6503 | 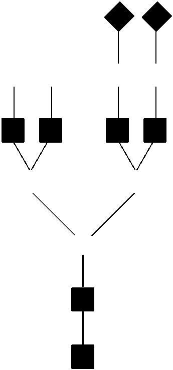 | Hex(6)HexNAc(5)Fuc(0)NeuAc(3) |
| 6513 | 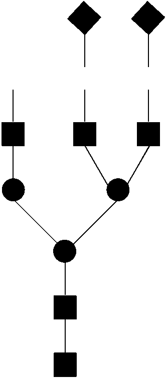 | Hex(6)HexNAc(5)Fuc(1)NeuAc(3) |
| 6521 | 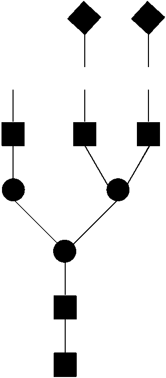 | Hex(6)HexNAc(5)Fuc(1)NeuAc(3) |
| 7602 | 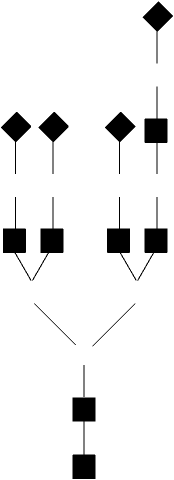 | Hex(7)HexNAc(6)Fuc(0)NeuAc(2) |
| 7603 | 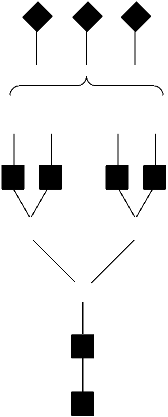 | Hex(7)HexNAc(6)Fuc(0)NeuAc(3) |
| 7612 | 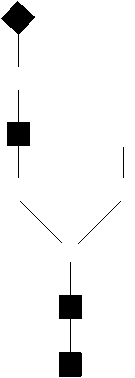 | Hex(7)HexNAc(6)Fuc(1)Neu(5)Ac(2) |
| 7614 | [symbol] | Hex(7)HexNAc(6)Fuc(1)NeuAc(4) |
| 8704 | 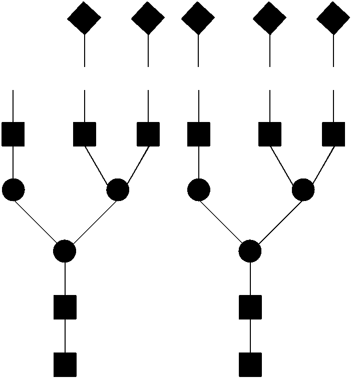 | Hex(8)HexNAc(7)Fuc(0)NeuAc(4) |
| 9804 | 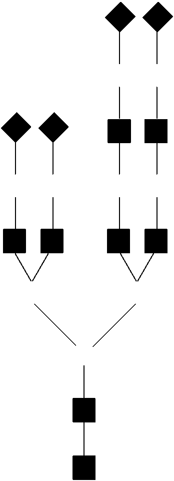 | Hex(9)HexNAc(8)Fuc(0)NeuAc(4) |
| 121005 | 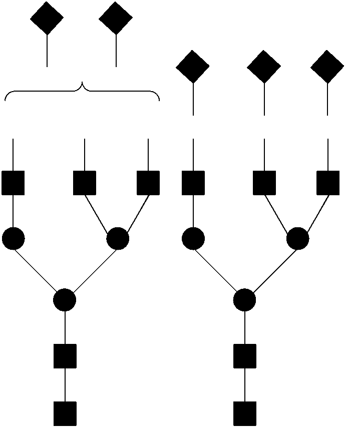 | Hex(12)HexNAc(10)Fuc(0)NeuAc(5) |

Legend for Table 7A

| | | | | |
|---|---|---|---|---|
| [symbol] | [symbol] | [symbol] | [symbol] | [symbol] |
| Glc | Gal | Man | Fuc | Neu5Ac |
| [symbol] | [symbol] | [symbol] | [symbol] | [symbol] |
| GlcNAc | GalNAc | ManNAc | Xyl | Neu5Gc |
| [symbol] | [symbol] | [symbol] | | [symbol] |
| GlcN | GalN | ManN | | Kdn |
| [symbol] | [symbol] | [symbol] | [symbol] | |
| GlcA | GalA | ManA | IdoA | |

Table 7A illustrates the symbol structure and composition of detected glycan moieties that correspond to glycopeptides of Tables 1A-3A based on the Glycan GL NO. The term Symbol Structure illustrates a geometric linking structure of the carbohydrates where the bottommost carbohydrate is bound to the designated amino acid for an N-linked glycan and the rightmost carbohydrate is bound to the designated amino acid for an O-linked glycan.

The identity of the various monosaccharides is illustrated by the Legend section located at the end of Table 7A. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an ovarian cancer disease state. A transition includes a precursor ion and at least one product ion grouping. As reviewed herein, the peptide structures in Tables 1A, 2A, and 3A, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 20. The alkylation procedure may be implemented in a manner similar to, for example, alkylation procedure 204 in FIG. 20. The digestion procedure may be implemented in a manner similar to, for example, digestion procedure 206 in FIG. 20.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 4A or an m/z ratio within an identified m/z ratio as provided in Table 4A. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VII. EMBODIMENTS

1. A method of detecting one or more multiple-reaction-monitoring (MRM) transitions, comprising:
obtaining, or having obtained, a biological sample from a patient, wherein the biological sample comprises one or more glycans or glycopeptides;
digesting and/or fragmenting a glycopeptide in the sample; and
detecting a MRM transition selected from the group consisting of transitions 1-38 from Tables 1-3.

2. The method of embodiment 1, wherein fragmenting the glycopeptide in the sample occurs after introducing the sample, or a portion thereof, into a mass spectrometer.

3. The method of any one of embodiments 1 or 2, wherein fragmenting the glycopeptide in the sample produces a glycopeptide ion, a peptide ion, a glycan ion, a glycan adduct ion, or a glycan fragment ion.

4. The method of any one of embodiments 1-3, wherein digesting the glycopeptide in the sample produces a peptide or glycopeptide consisting essentially of an amino acid having a sequence selected from the group consisting of SEQ ID NOs: 1-38, and combinations thereof.

5. The method of any one of embodiments 1-4, wherein the MRM transition is selected from the transitions, or any combinations thereof, in any one of Tables 1-3.

6. The method of any one of embodiments 1-5, further comprising conducting tandem liquid chromatography-mass spectroscopy on the biological sample.

7. The method of any one of embodiments 1-6, wherein detecting a MRM transition selected from the group consisting of transitions 1-38 comprises conducting multiple-reaction-monitoring mass spectroscopy (MRM-MS) mass spectroscopy on the biological sample.

8. The method of any one of embodiments 1-3 or 5-7, wherein the one or more glycopeptides comprises a peptide or glycopeptide:
consisting essentially of an amino acid sequence selected from the group consisting of SEQ ID NOs: 1-38, and combinations thereof;

9. The method of any one of embodiments 1-8, comprising detecting one or more MRM transitions indicative of one or more glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof.

10. The method of embodiment 9, further comprising quantifying a first glycan and quantifying a second glycan; and further comprising comparing the quantification of the first glycan with the quantification of the second glycan.

11. The method of embodiment 9 or 10, further comprising associating the detected glycan with a peptide residue site, whence the glycan was bonded.

12. The method of embodiment 11, further comprising quantifying relative abundance of a glycan and/or a peptide.

13. The method of any one of embodiments 1-12, comprising normalizing the amount of glycopeptide based on the amount of peptide or glycopeptide consisting essentially of an amino acid having a SEQ ID. No: 1-38.

14. A method for identifying a classification for a sample, the method comprising
quantifying by mass spectroscopy (MS) one or more glycopeptides in a sample wherein the glycopeptides each, individually in each instance, comprises a glycopeptide consisting essentially of an amino acid sequence selected from the group consisting of SEQ ID NOs: 1-38, and combinations thereof; and
inputting the quantification into a trained model to generate a output probability;
determining if the output probability is above or below a threshold for a classification; and
identifying a classification for the sample based on whether the output probability is above or below a threshold for a classification.

15. The method of embodiment 14, wherein the sample is a biological sample from a patient or individual having a disease or condition.

16. The method of embodiment 15, wherein the patient has cancer, an autoimmune disease, or fibrosis.

17. The method of embodiment 15, wherein the patient has ovarian cancer.

18. The method of embodiment 15, wherein the individual has an aging condition.

19. The method of embodiment 15, wherein the disease or condition is ovarian cancer.

20. The method of embodiment any one of embodiments 14-19, wherein the trained model was trained used a machine learning system selected from the group consisting of a deep learning system, a neural network system, an artificial neural network system, a supervised machine learning system, a linear discriminant analysis system, a quadratic discriminant analysis system, a support vector machine system, a linear basis function kernel support vector system, a radial basis function kernel support vector system, a random forest system, a genetic system, a nearest neighbor system, k-nearest neighbors, a naive Bayes classifier system, a logistic regression system, or a combination thereof.

21. The method of embodiment any one of embodiments 14-20, wherein the classification is a disease classification or a disease severity classification.

22. The method of embodiment 21, wherein the classification is identified with greater than 80% confidence, greater than 85% confidence, greater than 90% confidence, greater than 95% confidence, greater than 99% confidence, or greater than 99.9999% confidence.

23. The method of embodiment any one of embodiments 11-22, further comprising:
quantifying by MS a first glycopeptide in a sample at a first time point;
quantifying by MS a second glycopeptide in a sample at a second time point; and
comparing the quantification at the first time point with the quantification at the second time point.

24. The method of embodiment 23, further comprising:
quantifying by MS a third glycopeptide in a sample at a third time point;
quantifying by MS a fourth glycopeptide in a sample at a fourth time point; and
comparing the quantification at the fourth time point with the quantification at the third time point.

25. The method of any one of embodiments 14-24, further comprising monitoring the health status of a patient.

26. The method of any one of embodiments 14-25, further comprising quantifying by MS a glycopeptide from whence the amino acid sequence selected from the group consisting of SEQ ID NOs: 1-38 was fragmented.

27. The method of any one of embodiments 14-26, further comprising diagnosing a patient with a disease or condition based on the classification.

28. The method of embodiment 27, further comprising diagnosing the patient as having ovarian cancer based on the classification.

29. The method of any one of embodiments 14-28, further comprising treating the patient with a therapeutically effective amount of a therapeutic agent selected from the group consisting of a chemotherapeutic, an immunotherapy, a hormone therapy, a targeted therapy, and combinations thereof.

30. A method for treating a patient having ovarian cancer; the method comprising:
obtaining, or having obtained, a biological sample from the patient;
digesting and/or fragmenting, or having digested or having fragmented, one or more glycopeptides in the sample; and
detecting and quantifying one or more multiple-reaction-monitoring (MRM) transitions selected from the group consisting of transitions 1-38;
inputting the quantification into a trained model to generate an output probability;
determining if the output probability is above or below a threshold for a classification; and
classifying the patient based on whether the output probability is above or below a threshold for a classification, wherein the classification is selected from the group consisting of:
   (A) a patient in need of a chemotherapeutic agent;
   (B) a patient in need of a immunotherapeutic agent;
   (C) a patient in need of hormone therapy;
   (D) a patient in need of a targeted therapeutic agent;
   (E) a patient in need of surgery;
   (F) a patient in need of neoadjuvant therapy;
   (G) a patient in need of chemotherapeutic agent, immunotherapeutic agent, hormone therapy, targeted therapeutic agent, neoadjuvant therapy, or a combination thereof, before surgery;
   (H) a patient in need of chemotherapeutic agent, immunotherapeutic agent, hormone therapy, targeted therapeutic agent, neoadjuvant therapy, or a combination thereof, after surgery;
   (I) or a combination thereof;
      administering a therapeutically effective amount of a therapeutic agent to the patient:
wherein the therapeutic agent is selected from chemotherapy if classification A or I is determined;
wherein the therapeutic agent is selected from immunotherapy if classification B or I is determined; or
wherein the therapeutic agent is selected from hormone therapy if classification C or I is determined; or
wherein the therapeutic agent is selected from targeted therapy if classification D or I is determined
wherein the therapeutic agent is selected from neoadjuvant therapy if classification F or I is determined;
wherein the therapeutic agent is selected from chemotherapeutic agent, immunotherapeutic agent, hormone therapy, targeted therapeutic agent, neoadjuvant therapy, or a combination thereof if classification G or I is determined; and
wherein the therapeutic agent is selected from chemotherapeutic agent, immunotherapeutic agent, hormone therapy, targeted therapeutic agent, neoadjuvant therapy, or a combination thereof if classification H or I is determined.

31. The method of embodiment 30, comprising conducting multiple-reaction-monitoring mass spectroscopy (MRM-MS) on the biological sample.

32. The method of any one of embodiments 30-31, wherein the analyzing the transitions comprises selecting peaks and/or quantifying detected glycopeptide fragments with a machine learning system.

33. A method for diagnosing a patient having ovarian cancer; the method comprising:
obtaining, or having obtained, a biological sample from the patient;
performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect one or more MRM transitions selected from transitions 1-38;
inputting the quantification of the detected glycopeptides or the MRM transitions into a trained model to generate an output probability,
determining if the output probability is above or below a threshold for a classification; and
identifying a diagnostic classification for the patient based on whether the output probability is above or below a threshold for a classification; and
diagnosing the patient as having ovarian cancer based on the diagnostic classification.

34. The method of embodiment 33, wherein the analyzing the detected glycopeptides comprises using a machine learning system.

35. A glycopeptide consisting of an amino acid sequence selected from the group consisting of SEQ ID NOs: 1-38, and combinations thereof.

36. A glycopeptide consisting essentially an amino acid sequence selected from the group consisting essentially of SEQ ID NOs: 1-38, and combinations thereof.

37. A kit comprising a glycopeptide standard, a buffer, and one or more glycopeptides consisting essentially of an amino acid sequence selected from the group consisting of SEQ ID NOs: 1-38.

1A. A method for diagnosing a subject with respect to an ovarian cancer disease state, the method comprising:
receiving peptide structure data corresponding to a biological sample obtained from the subject;
analyzing the peptide structure data using a supervised machine learning model to generate a disease indicator that indicates whether the biological sample evidences an ovarian cancer disease state based on at least three peptide structures selected from one of a first group of peptide structures identified in Table 1A and a second group of peptide structures identified in Table 2A,
   wherein the first group of peptide structures and the second group of peptide structures are associated with the ovarian cancer disease state;
   wherein each of the first group of peptide structures in Table 1A and the second group of peptide structures in Table 2A is listed in order of relative significance to the disease indicator; and
      generating a diagnosis output based on the disease indicator.

2A. The method of embodiment 1A, wherein the disease indicator comprises a score.

3A. The method of embodiment 2A, wherein generating the diagnosis output comprises:
determining that the score falls above a selected threshold; and
generating the diagnosis output based on the score falling above the selected threshold, wherein the diagnosis output includes a positive diagnosis for the ovarian cancer disease state.

4A. The method of embodiment 2A, wherein generating the diagnosis output comprises:
determining that the score falls below a selected threshold; and
generating the diagnosis output based on the score falling below the selected threshold, wherein the diagnosis output includes a negative diagnosis for the ovarian cancer disease state.

5A. The method of embodiment 3A or embodiment 4A, wherein the score comprises a probability score and the selected threshold is 0.5.

6A. The method of embodiment 3A or embodiment 4A, wherein the selected threshold falls within a range between 0.30 and 0.65.

7A. The method of any one of embodiments 1A-6A, wherein analyzing the peptide structure data comprises:
analyzing the peptide structure data using a binary classification model.

8A. The method of any one of embodiments 1A-7A, wherein a peptide structure of the at least three peptide structures comprises a glycopeptide structure defined by a peptide sequence and a glycan structure linked to the peptide sequence at a linking site of the peptide sequence, as identified in Table 1A or Table 2A, with the peptide sequence being one of SEQ ID NOS: 111-119 in Table 1A as defined in Table 5A or one of SEQ ID NOS: 114, 115, and 131-146 in Table 2A as defined in Table 5A.

9A. The method of any one of embodiments 1A-8A, further comprising:
training the supervised machine learning model using training data,
wherein the training data comprises a plurality of peptide structure profiles for a plurality of subjects and a plurality of subject diagnoses for the plurality of subjects.

10A. The method of embodiment 9A, wherein the plurality of subject diagnoses includes a positive diagnosis for any subject of the plurality of subjects determined to have the ovarian cancer disease state and a negative diagnosis for any subject of the plurality of subjects determined to have a healthy state.

11A. The method of embodiment 9A, wherein the plurality of subject diagnoses includes a positive diagnosis for any subject of the plurality of subjects determined to have the ovarian cancer disease state and a negative diagnosis for any subject of the plurality of subjects determined to have a benign tumor state.

12A. The method of any one of embodiments 9A-11A, further comprising:
performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state; and
identifying a training group of peptide structures based on the differential expression analysis for use as prognostic markers for the ovarian cancer disease state; and
forming the training data based on the training group of peptide structures identified.

13A. The method of embodiment 12A, wherein training the supervised machine learning model comprises reducing the training group of peptide structures to a final group of peptide structures identified in Table 1A.

14A. The method of embodiment 12A, wherein training the supervised machine learning model comprises reducing the training group of peptide structures to a final group of peptide structures identified in Table 2A.

15A. The method of any one of embodiments 9A-14A, wherein each peptide structure profile of the plurality of peptide structure profiles includes a feature selected from one of a relative abundance and a concentration for a corresponding peptide structure.

16A. The method of any one of embodiments 9A-15A, wherein the plurality of peptide structure profiles includes a first peptide structure profile with a relative abundance for a corresponding peptide structure and a second peptide structure profile with a concentration for the corresponding peptide structure.

17A. The method of any one of embodiments 1A-16A, wherein the supervised machine learning model comprises a logistic regression model.

18A. The method of any one of embodiments 1A-17A, wherein the first group of peptide structures in Table 1A is used to distinguish between the ovarian cancer disease state and a healthy state and wherein the second group of peptide structures in Table 2A is used to distinguish between the ovarian cancer disease state and a benign tumor state.

19A. The method of any one of embodiments 1A-18A, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

20A. The method of any one of embodiments 1A-19A, wherein the peptide structure data is generated using multiple reaction monitoring mass spectrometry (MRM-MS).

21A. The method of any one of embodiments 1A-20A, further comprising:
preparing a sample of the biological sample using reduction, alkylation, and enzymatic digestion to form a prepared sample that includes a set of peptide structures.

22A. The method of embodiment 21A, further comprising:
generating the peptide structure data from the prepared sample using multiple reaction monitoring mass spectrometry (MRM-MS).

23A. The method of any one of embodiments 1A-22A, wherein generating the diagnosis output comprises:
generating a report identifying that the biological sample evidences the ovarian cancer disease state.

24A. The method of any one of embodiments 1A-23A, further comprising:
generating a treatment output based on at least one of the diagnosis output or the disease indicator.

25A. The method of embodiment 24A, wherein the treatment output comprises at least one of an identification of a treatment to treat the subject or a treatment plan.

26A. The method of embodiment 25A, wherein the treatment comprises at least one of surgery, radiation therapy, a targeted drug therapy, chemotherapy, immunotherapy, hormone therapy, or neoadjuvant therapy.

27A. A method of training a model to diagnose a subject with respect to an ovarian cancer disease state, the method comprising:
receiving quantification data for a panel of peptide structures for a plurality of samples for a plurality of subjects,
   wherein the plurality of subjects includes a first portion diagnosed with a negative diagnosis of an ovarian cancer disease state and a second portion diagnosed with a positive diagnosis of the ovarian cancer disease state;
   wherein the quantification data comprises a plurality of peptide structure profiles for the plurality of subjects; and
training a machine learning model using the quantification data to diagnose a biological sample with respect to the ovarian cancer disease state using a first group of peptide structures associated with the ovarian cancer disease state or a second group of peptide structures associated with the ovarian cancer disease state,
   wherein the first group of peptide structures is identified in Table 1A and listed in Table 1A with respect to relative significance to diagnosing the biological sample; and
   wherein the second group of peptide structures is identified in Table 2A and listed in Table 2A with respect to relative significance to diagnosing the biological sample.

28A. The method of embodiment 27A, wherein the machine learning model comprises a logistic regression model.

29A. The method of embodiment 28A, wherein the logistic regression model comprises a LASSO regression model.

30A. The method of any one of embodiments 27A-29A, further comprising:
identifying an initial plurality of peptide structure profiles;
filtering the initial plurality of peptide structure profiles by a coefficient of variation to generate a plurality of peptide structure profiles for use in training the machine learning model.

31A. The method of embodiment 30A, wherein the filtering is performed to exclude peptide structure profiles having the coefficient of variation at or above 20%.

32A. The method of embodiment 30A, wherein training the machine learning model comprises reducing the plurality of peptide structure profiles using LASSO regression to identify a final group of peptide structures identified in Table 1A.

33A. The method of embodiment 30A, wherein training the machine learning model comprises reducing the plurality of peptide structure profiles using LASSO regression to identify a final group of peptide structures identified in Table 2A.

34A. The method of any one of embodiments 27A-33A, wherein the negative diagnosis for the ovarian cancer disease state indicates a healthy state.

35A. The method of any one of embodiments 27A-34A, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

36A. The method of any one of embodiments 27A-35A, wherein the ovarian cancer disease state includes a malignant pelvic tumor.

37A. The method of any one of embodiments 27A-36A, wherein the ovarian cancer disease state is epithelial ovarian cancer.

38A. The method of any one of embodiments 27A-33A, wherein the negative diagnosis for the ovarian cancer disease state indicates a benign pelvic tumor.

39A. The method of any one of embodiments 27A-38A, wherein the trained model uses a relative abundance for a first portion of the first group of peptide structures and a concentration for a second portion of the second group of peptide structures.

40A. The method of any one of embodiments 27A-39A, wherein the training comprises:

identifying a first portion of the plurality of samples for subjects with benign pelvic tumors and malignant pelvic tumors and a second portion of the plurality of samples for subjects with a healthy status; and
generating a training set of peptide structure profiles for 80% of the first portion and a test set of peptide structure profiles for a remaining 20% of the first portion and the second portion.

41A. A composition comprising at least one of peptide structures PS-1-PS-10 identified in Table 1A.

42A. A composition comprising at least one of peptide structures PS-11-PS-34 and PS-5 identified in Table 2A.

43A. A composition comprising at least one of peptide structures PS-1-PS-10 and PS-11-PS-34 from Table 1A and Table 2A.

44A. A composition comprising a peptide structure or a product ion, wherein:
the peptide structure or the product ion comprises an amino acid sequence having at least 90% sequence identity to any one of SEQ ID NOS: 111-119, corresponding to respective ones of peptide structures PS-1 to PS-10 in Table 1A; and
the product ion is selected as one from a group consisting of product ions corresponding to PS-1 to PS-10 identified in Table 4A including product ions falling within an identified m/z range.

45A. A composition comprising a peptide structure or a product ion, wherein:
the peptide structure or the product ion comprises an amino acid sequence having at least 90% sequence identity to any one of SEQ ID NOS: 114, 115, and 131-146 corresponding to respective ones of peptide structures PS-5 and PS-11-PS-34 in Table 2A; and
the product ion is selected as one from a group consisting of product ions corresponding to PS-5 and PS-11-PS-34 identified in Table 2A including product ions falling within an identified m/z range.

46A. A composition comprising a peptide structure or a product ion, wherein:
the peptide structure or the product ion comprises an amino acid sequence having at least 90% sequence identity to SEQ ID NO: 115, corresponding to peptide structure PS-5 in Tables 1A, 2A, and 3A; and
the product ion is selected as one from a group consisting of product ions corresponding to PS-5 identified in Table 4A including product ions falling within an identified m/z range.

47A. A composition comprising a glycopeptide structure selected as one from a group consisting of peptide structures PS-1 to PS-10 identified in Table 1A, wherein:
the peptide structure comprises:
   an amino acid peptide sequence identified in Table 5A as corresponding to the peptide structure; and
   a glycan structure identified in Table 7A as corresponding to the peptide structure in which the glycan structure is linked to a residue of the amino acid peptide sequence at a corresponding position identified in Table 1A; and wherein the glycan structure has a glycan composition.

48A. A composition comprising a glycopeptide structure selected as one from a group consisting of peptide structures PS-5 and PS-11-PS-34 identified in Table 2A, wherein: the peptide structure comprises:
an amino acid peptide sequence identified in Table 5A as corresponding to the peptide structure; and
a glycan structure identified in Table 7A as corresponding to the peptide structure in which the glycan structure is linked to a residue of the amino acid peptide sequence at a corresponding position identified in Table 2A; and wherein the glycan structure has a glycan composition.

49A. The composition of any one of embodiments 47A-48A, wherein the glycan composition is identified in Table 7A.

50A. The composition of any one of embodiments 47A-49A, wherein:
the peptide structure has a precursor ion having a charge identified in Table 4A as corresponding to the peptide structure.

51A. The composition of any one of embodiments 47A-50A, wherein:
the peptide structure has a precursor ion with an m/z ratio within ±1.5 of the m/z ratio listed for the precursor ion in Table 4A as corresponding to the glycopeptide structure.

52A. The composition of any one of embodiments 47A-50A, wherein:
the peptide structure has a precursor ion with an m/z ratio within ±1.0 of the m/z ratio listed for the precursor ion in Table 4A as corresponding to the peptide structure.

53A. The composition of any one of embodiments 47A-50A, wherein:
the peptide structure has a precursor ion with an m/z ratio within ±0.5 of the m/z ratio listed for the precursor ion in Table 4A as corresponding to the peptide structure.

54A. The composition of any one of embodiments 47A-53A, wherein:
the peptide structure has a product ion with an m/z ratio within ±1.0 of the m/z ratio listed for the product ion in Table 4A as corresponding to the peptide structure.

55A. The composition of any one of embodiments 47A-53A, wherein:
the peptide structure has a product ion with an m/z ratio within ±0.8 of the m/z ratio listed for the product ion in Table 4A as corresponding to the peptide structure.

56A. The composition of any one of embodiments 47A-53A, wherein:
the peptide structure has a product ion with an m/z ratio within ±0.5 of the m/z ratio listed for the product ion in Table 4A as corresponding to the peptide structure.

57A. The composition of any one of embodiments 47A-56A, wherein the peptide structure has a monoisotopic mass identified in Table 1A as corresponding to the peptide structure.

58A. The composition of any one of embodiments 47A-56A, wherein the peptide structure has a monoisotopic mass identified in Table 2A as corresponding to the peptide structure.

59A. A composition comprising a peptide structure selected as one from a plurality of peptide structures identified in Table 1A, wherein:
the peptide structure has a monoisotopic mass identified as corresponding to the peptide structure in Table 1A; and
the peptide structure comprises the amino acid sequence of SEQ ID NOs: 111-119 identified in Table 1A as corresponding to the peptide structure.

60A. A composition comprising a peptide structure selected as one from a plurality of peptide structures identified in Table 2A, wherein:
the peptide structure has a monoisotopic mass identified as corresponding to the peptide structure in Table 2A; and
the peptide structure comprises the amino acid sequence of SEQ ID NOS: 114, 115, 131-146 identified in Table 2A as corresponding to the peptide structure.

61A. The composition of any one of embodiments 59A-60A, wherein:

the peptide structure has a precursor ion having a charge identified in Table 4A as corresponding to the peptide structure.

62A. The composition of any one of embodiments 59A-61A, wherein:
the peptide structure has a precursor ion with an m/z ratio within ±1.5 of the m/z ratio listed for the precursor ion in Table 4A as corresponding to the peptide structure.

63A. The composition of any one of embodiments 59A-61A, wherein:
the peptide structure has a precursor ion with an m/z ratio within ±1.0 of the m/z ratio listed for the precursor ion in Table 4A as corresponding to the peptide structure.

64A. The composition of any one of embodiments 59A-61A, wherein:
the peptide structure has a precursor ion with an m/z ratio within ±0.5 of the m/z ratio listed for the precursor ion in Table 4A as corresponding to the peptide structure.

65A. The composition of any one of embodiments 59A-64A, wherein:
the peptide structure has a product ion with an m/z ratio within ±1.0 of the m/z ratio listed for the product ion in Table 4A as corresponding to the peptide structure.

66A. The composition of any one of embodiments 59A-64A, wherein:
the peptide structure has a product ion with an m/z ratio within ±0.8 of the m/z ratio listed for the product ion in Table 4A as corresponding to the peptide structure.

67A. The composition of any one of embodiments 59A-64A, wherein:
the peptide structure has a product ion with an m/z ratio within ±0.5 of the m/z ratio listed for the product ion in Table 4A as corresponding to the peptide structure.

68A. A kit comprising at least one agent for quantifying at least one peptide structure identified in Table 1A to carry out the method of any one of embodiments 1A-40A.

69A. A kit comprising at least one agent for quantifying at least one peptide structure identified in Table 2A to carry out the method of any one of embodiments 1A-40A.

70A. A kit comprising at least one agent for quantifying at least one peptide structure identified in at least one of Table 1A or Table 2A to carry out the method of any one of embodiments 1A-40A.

71A. A kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 111-119, defined in Table 1A and Table 5A.

72A. A kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 114, 115, and 131-146, defined in Table 2A and Table 5A.

73A. A kit comprising at least one of a glycopeptide standard, a buffer, or a set of peptide sequences to carry out the method of any one of embodiments 1A-40A, a peptide sequence of the set of peptide sequences identified by a corresponding one of SEQ ID NOS: 111-119 and 131-146 defined in Tables 1A, 2A, and 5A.

74A. A system comprising:

one or more data processors; and
a non-transitory computer readable storage medium containing instructions which, when executed on the one or more data processors, cause the one or more data processors to perform part or all of any one of embodiments 1A-40A.

75A. A computer-program product tangibly embodied in a non-transitory machine-readable storage medium, including instructions configured to cause one or more data processors to perform part or all of any one of embodiments 1A-40A.

76A. A method for diagnosing a subject with respect to an ovarian cancer disease state, the method comprising:
receiving peptide structure data corresponding to a biological sample obtained from the subject;
analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 3A,

wherein the group of peptide structures in Table 3A is listed in order of relative significance to the disease indicator; and

generating a diagnosis output based on the disease indicator.

77A. The method of embodiment 76A, wherein the disease indicator comprises a score.

78A. The method of embodiment 77A, wherein generating the diagnosis output comprises:
determining that the score falls above a selected threshold; and
generating the diagnosis output based on the score falling above the selected threshold, wherein the diagnosis output includes a positive diagnosis for the ovarian cancer disease state.

79A. The method of embodiment 77A, wherein generating the diagnosis output comprises:
determining that the score falls below a selected threshold; and
generating the diagnosis output based on the score falling below the selected threshold, wherein the diagnosis output includes a negative diagnosis for the ovarian cancer disease state.

80A. The method of embodiment 78A or embodiment 79A, wherein the score comprises a probability score and the selected threshold is 0.5.

81A. The method of embodiment 78A or embodiment 79A, wherein the selected threshold falls within a range between 0.30 and 0.65.

82A. The method of any one of embodiments 76A-81A, wherein analyzing the peptide structure data comprises:
analyzing the peptide structure data using a binary classification model.

83A. The method of any one of embodiments 76A-82A, wherein a peptide structure of the at least three peptide structures comprises a glycopeptide structure defined by a peptide sequence and a glycan structure linked to the peptide sequence at a linking site of the peptide sequence, as identified in Table 3A, with the peptide sequence being one of SEQ ID NOS: 111, 114, 115, 131, 132, 133, 134, 137, 138, 140, 142, 144, 145, 146, 153-165 in Table 3A as defined in Table 5A.

84A. The method of any one of embodiments 76A-83A, further comprising:
training the supervised machine learning model using training data,
wherein the training data comprises a plurality of peptide structure profiles for a plurality of subjects and a plurality of subject diagnoses for the plurality of subjects.

85A. The method of embodiment 84A, wherein the plurality of subject diagnoses includes a positive diagnosis for any subject of the plurality of subjects determined to have the malignant pelvic tumor and a negative diagnosis for any subject of the plurality of subjects determined to have a healthy state.

86A. The method of embodiment 84A, wherein the plurality of subject diagnoses includes a positive diagnosis for any subject of the plurality of subjects determined to have the ovarian cancer disease state and a negative diagnosis for any subject of the plurality of subjects determined to have a benign pelvic tumor.

87A. The method of any one of embodiments 84A-86A, further comprising:
performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state; and
identifying a training group of peptide structures based on the differential expression analysis for use as prognostic markers for the ovarian cancer disease state; and
forming the training data based on the training group of peptide structures identified.

88A. The method of embodiment 87A, wherein training the supervised machine learning model comprises reducing the training group of peptide structures to a final group of peptide structures identified in Table 3A.

89A. The method of any one of embodiments 84A-88A, wherein each peptide structure profile of the plurality of peptide structure profiles includes a feature selected from one of a relative abundance and a concentration for a corresponding peptide structure.

90A. The method of any one of embodiments 84A-89A, wherein the plurality of peptide structure profiles includes a first peptide structure profile with a relative abundance for a corresponding peptide structure and a second peptide structure profile with a concentration for the corresponding peptide structure.

91A. The method of any one of embodiments 76A-90A, wherein the supervised machine learning model comprises a logistic regression model.

92A. The method of any one of embodiments 76A-91A, wherein the first group of peptide structures in Table 3A is used to distinguish between the ovarian cancer disease state having the malignant pelvic tumor and a non-ovarian cancer state having a benign pelvic tumor.

93A. The method of any one of embodiments 76A-92A, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

94A. The method of any one of embodiments 76A-93A, wherein the peptide structure data is generated using multiple reaction monitoring mass spectrometry (MRM-MS).

95A. The method of any one of embodiments 76A-94A, further comprising:
preparing a sample of the biological sample using reduction, alkylation, and enzymatic digestion to form a prepared sample that includes a set of peptide structures.

96A. The method of embodiment 95A, further comprising:
generating the peptide structure data from the prepared sample using multiple reaction monitoring mass spectrometry (MRM-MS).

97A. The method of any one of embodiments 76A-96A, wherein generating the diagnosis output comprises:
generating a report identifying that the biological sample evidences the ovarian cancer disease state.

98A. The method of any one of embodiments 76A-97A, further comprising:
generating a treatment output based on at least one of the diagnosis output or the disease indicator.

99A. The method of embodiment 98A, wherein the treatment output comprises at least one of an identification of a treatment to treat the subject or a treatment plan.

100A. The method of embodiment 99A, wherein the treatment comprises at least one of surgery, radiation therapy, a targeted drug therapy, chemotherapy, immunotherapy, hormone therapy, or neoadjuvant therapy.

101A. A method of training a model to diagnose a subject with respect to an ovarian cancer disease state having a malignant pelvic tumor, the method comprising:
receiving quantification data for a panel of peptide structures for a plurality of samples for a plurality of subjects,
   wherein the plurality of subjects includes a first portion diagnosed with a negative diagnosis of an ovarian cancer disease state and a second portion diagnosed with a positive diagnosis of the ovarian cancer disease state;
   wherein the quantification data comprises a plurality of peptide structure profiles for the plurality of subjects; and
training a machine learning model using the quantification data to diagnose a biological sample with respect to the ovarian cancer disease state using a group of peptide structures associated with the ovarian cancer disease state,
   wherein the group of peptide structures is identified in Table 3A and listed in Table 3A with respect to relative significance to diagnosing the biological sample.

102A. The method of embodiment 101A, wherein the machine learning model comprises a logistic regression model.

103A. The method of embodiment 102A, wherein the logistic regression model comprises a LASSO regression model.

104A. The method of any one of embodiments 101A-102A, further comprising:
identifying an initial plurality of peptide structure profiles;
filtering the initial plurality of peptide structure profiles by a coefficient of variation to generate a plurality of peptide structure profiles for use in training the machine learning model.

105A. The method of embodiment 104A, wherein the filtering is performed to exclude peptide structure profiles having the coefficient of variation at or above 20%.

106A. The method of embodiment 104A, wherein training the machine learning model comprises reducing the plurality of peptide structure profiles using LASSO regression to identify a final group of peptide structures identified in Table 3A.

107A. The method of any one of embodiments 101A-106A, wherein the negative diagnosis for the ovarian cancer disease state indicates a non-ovarian cancer state comprising a benign tumor state.

108A. The method of any one of embodiments 101A-107A, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

109A. The method of any one of embodiments 101A-108A, wherein the trained model uses a relative abundance for a first portion of the first group of peptide structures and a concentration for a second portion of the second group of peptide structures.

110A. The method of any one of embodiments 101A-109A, wherein the training comprises:

identifying a first portion of the plurality of samples for subjects with benign pelvic tumors and malignant pelvic tumors and a second portion of the plurality of samples for subjects with a healthy status; and
generating a training set of peptide structure profiles for 80% of the first portion and a test set of peptide structure profiles for a remaining 20% of the first portion and the second portion.

111A. A composition comprising at least one of peptide structures PS-1, PS-5, PS-11, PS-15, PS-20, PS-25, PS-28, PS-29, PS-30, PS-31, PS-32, and PS-35 to PS-61 identified in Table 3A.

112A. A composition comprising at least one of peptide structures PS-1, PS-5, PS-11, PS-15, PS-20, PS-25, PS-28, PS-29, PS-30, PS-31, PS-32, or PS-35 to PS-61 identified in Table 3A and at least one of peptide structures PS-1-PS-34 in Tables 1A and 2A.

113A. A composition comprising a peptide structure or a product ion, wherein:
the peptide structure or the product ion comprises an amino acid sequence having at least 90% sequence identity to any one of SEQ ID NOS: 111, 114, 115, 131, 132, 133, 134, 137, 138, 140, 142, 144, 145, 146, 153-165 corresponding to respective ones of peptide structures PS-1, PS-5, PS-11, PS-15, PS-20, PS-25, PS-28, PS-29, PS-30, PS-31, PS-32, and PS-35 to PS-61 in Table 3A; and
the product ion is selected as one from a group consisting of product ions corresponding to PS PS-1, PS-5, PS-11, PS-15, PS-20, PS-25, PS-28, PS-29, PS-30, PS-31, PS-32, and PS-35 to PS-61 identified in Table 3A including product ions falling within an identified m/z range.

114A. A composition comprising a glycopeptide structure selected as one from a group consisting of peptide structures PS-1, PS-5, PS-11, PS-15, PS-20, PS-25, PS-28, PS-29, PS-30, PS-31, PS-32, and PS-35 to PS-61 identified in Table 3A, wherein:
the peptide structure comprises:
   an amino acid peptide sequence identified in Table 5A as corresponding to the peptide structure; and
   a glycan structure identified in Table 7A as corresponding to the peptide structure in which the glycan structure is linked to a residue of the amino acid peptide sequence at a corresponding position identified in Table 3A; and
   wherein the glycan structure has a glycan composition.

115A. The composition of embodiment 114A, wherein the glycan composition is identified in Table 7A.

116A. The composition of any one of embodiments 114A-115A, wherein:
the peptide structure has a precursor ion having a charge identified in Table 4A as corresponding to the peptide structure.

117A. The composition of any one of embodiments 114A-116A, wherein:
the peptide structure has a precursor ion with an m/z ratio within ±1.5 of the m/z ratio listed for the precursor ion in Table 4A as corresponding to the glycopeptide structure.

118A. The composition of any one of embodiments 114A-116A, wherein:
the peptide structure has a precursor ion with an m/z ratio within ±1.0 of the m/z ratio listed for the precursor ion in Table 4A as corresponding to the peptide structure.

119A. The composition of any one of embodiments 114A-116A, wherein:
the peptide structure has a precursor ion with an m/z ratio within ±0.5 of the m/z ratio listed for the precursor ion in Table 4A as corresponding to the peptide structure.

120A. The composition of any one of embodiments 114A-119A, wherein:
the peptide structure has a product ion with an m/z ratio within ±1.0 of the m/z ratio listed for the product ion in Table 4A as corresponding to the peptide structure.

121A. The composition of any one of embodiments 114A-119A, wherein:
the peptide structure has a product ion with an m/z ratio within ±0.8 of the m/z ratio listed for the product ion in Table 4A as corresponding to the peptide structure.

122A. The composition of any one of embodiments 114A-119A, wherein:
the peptide structure has a product ion with an m/z ratio within ±0.5 of the m/z ratio listed for the product ion in Table 4A as corresponding to the peptide structure.

123A. The composition of any one of embodiments 114A-122A, wherein the peptide structure has a monoisotopic mass identified in Table 3A as corresponding to the peptide structure.

124A. A composition comprising a peptide structure selected as one from a plurality of peptide structures identified in Table 3A, wherein:
the peptide structure has a monoisotopic mass identified as corresponding to the peptide structure in Table 3A; and
the peptide structure comprises the amino acid sequence of SEQ ID NOS: 111, 114, 115, 131, 132, 133, 134, 137, 138, 140, 142, 144, 145, 146, 153-165 identified in Table 3A as corresponding to the peptide structure.

125A. The composition of embodiment 124A, wherein:

the peptide structure has a precursor ion having a charge identified in Table 4A as corresponding to the peptide structure.

126A. The composition of any one of embodiments 124A-125A, wherein:

the peptide structure has a precursor ion with an m/z ratio within ±1.5 of the m/z ratio listed for
the precursor ion in Table 4A as corresponding to the peptide structure.

127A. The composition of any one of embodiments 124A-125A, wherein:
the peptide structure has a precursor ion with an m/z ratio within ±1.0 of the m/z ratio listed for the precursor ion in Table 4A as corresponding to the peptide structure.

128A. The composition of any one of embodiments 124A-125A, wherein:
the peptide structure has a precursor ion with an m/z ratio within ±0.5 of the m/z ratio listed for the precursor ion in Table 4A as corresponding to the peptide structure.

129A. The composition of any one of embodiments 124A-128A, wherein:
the peptide structure has a product ion with an m/z ratio within ±1.0 of the m/z ratio listed for the product ion in Table 4A as corresponding to the peptide structure.

130A. The composition of any one of embodiments 124A-128A, wherein:
the peptide structure has a product ion with an m/z ratio within ±0.8 of the m/z ratio listed for the product ion in Table 4A as corresponding to the peptide structure.

131A. The composition of any one of embodiments 124A-128A, wherein:
the peptide structure has a product ion with an m/z ratio within ±0.5 of the m/z ratio listed for the product ion in Table 4A as corresponding to the peptide structure.

132A. A kit comprising at least one agent for quantifying at least one peptide structure identified in Table 3A to carry out the method of any one of embodiments 76A-110A.

133A. A kit comprising at least one of a glycopeptide standard, a buffer, or a set of peptide sequences to carry out the method of any one of embodiments 76A-110A, a peptide sequence of the set of peptide sequences identified by a corresponding one of SEQ ID NOS: 111, 114, 115, 131, 132, 133, 134, 137, 138, 140, 142, 144, 145, 146, 153-165 identified in Table 3A.

134A. A system comprising:

one or more data processors; and
a non-transitory computer readable storage medium containing instructions which, when executed on the one or more data processors, cause the one or more data processors to perform part or all of any one of embodiments 76A-110A.

135A. A computer-program product tangibly embodied in a non-transitory machine-readable storage medium, including instructions configured to cause one or more data processors to perform part or all of any one of embodiments 76A-110A.

136A. The method of any one of embodiments 1A-26A, further comprising:
performing a biopsy of the subject in response to the diagnosis output indicating a positive diagnosis for the ovarian cancer disease state.

137A. The method of any one of embodiments 1A-26A, further comprising:
generating a report recommending that a biopsy be performed for the subject in response to the diagnosis output indicating a positive diagnosis for the ovarian cancer disease state.

138A. The method of any one of embodiments 27A-40A, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

139A. The method of any one of embodiments 27A-40A, further comprising:
generating, using the trained machine learning model, a disease indicator for diagnosing the biological sample with respect to the ovarian cancer disease state; and
generating a report recommending that a biopsy be performed for the subject in response to the diagnosis indicator indicating a positive diagnosis for the ovarian cancer disease state.

140A. The method of any one of embodiments 76A-100A, further comprising:
performing a biopsy of the subject in response to the diagnosis output indicating a positive diagnosis for the ovarian cancer disease state.

141A. The method of any one of embodiments 76A-100A, further comprising:
generating a report recommending that a biopsy be performed for the subject in response to the diagnosis output indicating a positive diagnosis for the ovarian cancer disease state.

142A. The method of any one of embodiments 101A-110A, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

143A. The method of any one of embodiments 101A-110A, further comprising:
generating, using the trained machine learning model, a disease indicator for diagnosing the biological sample with respect to the ovarian cancer disease state; and
   generating a report recommending that a biopsy be performed for the subject in response to the diagnosis indicator indicating a positive diagnosis for the ovarian cancer disease state.

1B. A method for diagnosing a subject with respect to an ovarian cancer disease state, the method comprising
receiving peptide structure data corresponding to a biological sample obtained from the subject;
analyzing the peptide structure data using a supervised machine learning model to generate a disease indicator that indicates whether the biological sample evidences an ovarian cancer disease state based on at least three peptide structures selected from one of a first group of peptide structures identified in Table 1A and a second group of peptide structures identified in Table 2A,
   wherein the first group of peptide structures and the second group of peptide structures are associated with the ovarian cancer disease state;
   wherein each of the first group of peptide structures in Table 1A and the second group of peptide structures in Table 2A is listed in order of relative significance to the disease indicator; and
      generating a diagnosis output based on the disease indicator.

2B. The method of embodiment 1B, wherein the disease indicator comprises a score.

3B. The method of embodiment 2B, wherein generating the diagnosis output comprises
determining that the score falls above a selected threshold; and
generating the diagnosis output based on the score falling above the selected threshold, wherein the diagnosis output includes a positive or negative diagnosis for the ovarian cancer disease state.

4B. The method of embodiment 3B, wherein the score comprises a probability score and the selected threshold is 0.5.

5B. The method of embodiment 3B or embodiment 4B, wherein the selected threshold falls within a range between 0.30 and 0.65.

6B. The method of any one of embodiments 1B-5B, wherein analyzing the peptide structure data comprises analyzing the peptide structure data using a binary classification model.

7B. The method of any one of embodiments 1B-6B, wherein a peptide structure of the at least three peptide structures comprises a glycopeptide structure defined by a peptide sequence and a glycan structure linked to the peptide sequence at a linking site of the peptide sequence, as identified in Table 1A or Table 2A, with the peptide sequence being one of SEQ ID NOS: 111-119 in Table 1A as defined in Table 5A or one of SEQ ID NOS: 114, 115, and 131-146 in Table 2A as defined in Table 5A.

8B. The method of any one of embodiments 1B-7B, further comprising:
training the supervised machine learning model using training data,
wherein the training data comprises a plurality of peptide structure profiles for a plurality of subjects and a plurality of subject diagnoses for the plurality of subjects.

9B. The method of embodiment 8B, wherein the plurality of subject diagnoses includes a positive diagnosis for any subject of the plurality of subjects determined to have the ovarian cancer disease state and a negative diagnosis for any subject of the plurality of subjects determined to have a healthy state or a benign tumor state.

10B. The method of any one of embodiments 8B-9B, wherein each peptide structure profile of the plurality of peptide structure profiles comprises a feature selected from one the group consisting of a relative abundance and a concentration for a corresponding peptide structure.

11B. The method of any one of embodiments 1B-10B, wherein the supervised machine learning model comprises a logistic regression model.

12B. The method of any one of embodiments 1B-11B, wherein the first group of peptide structures in Table 1A is used to distinguish between the ovarian cancer disease state and a healthy state and wherein the second group of peptide structures in Table 2A is used to distinguish between the ovarian cancer disease state and a benign tumor state.

13B. The method of any one of embodiments 1B-12B, wherein the peptide structure data comprises at least one of an abundance, a relative abundance, a normalized abundance, a relative quantity, an adjusted quantity, a normalized quantity, a relative concentration, an adjusted concentration, or a normalized concentration.

14B. A method of training a model to diagnose a subject with respect to an ovarian cancer disease state, the method comprising:
receiving quantification data for a panel of peptide structures for a plurality of biological samples for a plurality of subjects,
   wherein the plurality of subjects includes a first portion diagnosed with a negative diagnosis of an ovarian cancer disease state and a second portion diagnosed with a positive diagnosis of the ovarian cancer disease state;
   wherein the quantification data comprises a plurality of peptide structure profiles for the plurality of subjects; and
training a machine learning model using the quantification data to diagnose a biological sample with respect to the ovarian cancer disease state using a first group of peptide structures associated with the ovarian cancer disease state or a second group of peptide structures associated with the ovarian cancer disease state,
   wherein the first group of peptide structures is identified in Table 1A and listed in Table 1A with respect to relative significance to diagnosing the biological sample; and
   wherein the second group of peptide structures is identified in Table 2A and listed in Table 2A with respect to relative significance to diagnosing the biological sample.

15B. The method of embodiment 14B, wherein the machine learning model comprises a logistic regression model.

16B. The method of any one of embodiments 14B-15B, further comprising:
identifying an initial plurality of peptide structure profiles;
filtering the initial plurality of peptide structure profiles by a coefficient of variation to generate a plurality of peptide structure profiles for use in training the machine learning model.

17B. The method of embodiment 16B, wherein the filtering is performed to exclude peptide structure profiles having the coefficient of variation at or above 20%.

18B. The method of embodiment 14B, wherein training the machine learning model comprises reducing the plurality of peptide structure profiles using LASSO regression to identify a final group of peptide structures identified in Table 1A, or Table 2A.

19B. The method of any one of embodiments 14B-18B, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

20B. A method for diagnosing a subject with respect to an ovarian cancer disease state, the method comprising:
receiving peptide structure data corresponding to a biological sample obtained from the subject;
analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 3A; and
   generating a diagnosis output based on the disease indicator.

21B. The method of embodiment 20B, wherein the wherein the group of peptide structures in Table 3A is listed in order of relative significance to the disease indicator.

22B. The method of embodiment 20B or embodiment 21B, wherein the disease indicator comprises a score.

23B. The method of embodiment 22B, wherein generating the diagnosis output comprises:
determining that the score falls above a selected threshold; and
generating the diagnosis output based on the score falling above the selected threshold, wherein the diagnosis output includes a positive diagnosis for the ovarian cancer disease state.

24B. The method of embodiment 22B, wherein generating the diagnosis output comprises:
determining that the score falls below a selected threshold; and
generating the diagnosis output based on the score falling below the selected threshold, wherein the diagnosis output includes a negative diagnosis for the ovarian cancer disease state.

25B. The method of embodiment 23B or embodiment 24B, wherein the score comprises a probability score and the selected threshold is 0.5.

26B. The method of embodiment 23 B or embodiment 24 B, wherein the selected threshold falls within a range between 0.30 and 0.65.

27B. The method of any one of embodiments 20B-26B, wherein analyzing the peptide structure data comprises:
analyzing the peptide structure data using a binary classification model.

28B. The method of any one of embodiments 20B-27B, wherein a peptide structure of the at least three peptide structures comprises a glycopeptide structure defined by a peptide sequence and a glycan structure linked to the peptide sequence at a linking site of the peptide sequence, as identified in Table 3A, with the peptide sequence being one of SEQ ID NOS: 111, 114, 115, 131, 132, 133, 134, 137, 138, 140, 142, 144, 145, 146, 153-165.

29B. The method of embodiment 28B, wherein the peptide structure comprises an amino acid sequence set forth in SEQ ID NOS: 111, 114, 115, 131, 132, 133, 134, 137, 138, 140, 142, 144, 145, 146, or 153-165.

30B. The method of embodiment 28B or embodiment 29B, wherein the method comprises analyzing the peptide structure using a supervised machine learning model to generate a disease indicator that indicates whether the biological sample evidences the ovarian cancer disease state of having a malignant pelvic tumor based on at least five, at least 10 at least 15, at least 20, at least 25, at least 30, or at least 35 peptide structures selected from one of a group of peptide structures identified in Table 3A.

31B. The method of embodiment 30B, wherein the method comprises analyzing the peptide structure using a supervised machine learning model to generate a disease indicator that indicates whether the biological sample evidences the ovarian cancer disease state of having a malignant pelvic tumor based on each of the peptide structures selected from one of a group of peptide structures identified in Table 3A, comprising an amino acid sequence set forth in SEQ ID NOS: 111, 114, 115, 131, 132, 133, 134, 137, 138, 140, 142, 144, 145, 146, or 153-165.

32B. The method of any one of embodiments 20B-31B, further comprising:
training the supervised machine learning model using training data,
wherein the training data comprises a plurality of peptide structure profiles for a plurality of subjects and a plurality of subject diagnoses for the plurality of subjects.

33B. The method of embodiment 32B, wherein the plurality of subject diagnoses includes a positive diagnosis for any subject of the plurality of subjects determined to have the malignant pelvic tumor and a negative diagnosis for any subject of the plurality of subjects determined to have a healthy state.

34B. The method of embodiment 32B, wherein the plurality of subject diagnoses includes a positive diagnosis for any subject of the plurality of subjects determined to have the ovarian cancer disease state and a negative diagnosis for any subject of the plurality of subjects determined to have a benign pelvic tumor.

35B. The method of any one of embodiments 32B-34B, further comprising:
performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state; and
identifying a training group of peptide structures based on the differential expression analysis for use as prognostic markers for the ovarian cancer disease state; and
forming the training data based on the training group of peptide structures identified.

36B. The method of embodiment 35B, wherein training the supervised machine learning model comprises reducing the training group of peptide structures to a final group of peptide structures identified in Table 3A.

37B. The method of any one of embodiments 32B-36B, wherein each peptide structure profile of the plurality of peptide structure profiles includes a feature selected from one of a relative abundance and a concentration for a corresponding peptide structure.

38B. The method of any one of embodiments 32B-37B, wherein the plurality of peptide structure profiles includes a first peptide structure profile with a relative abundance for a corresponding peptide structure and a second peptide structure profile with a concentration for the corresponding peptide structure.

39B. The method of any one of embodiments 20B-38B, wherein the supervised machine learning model comprises a logistic regression model.

40B. The method of any one of embodiments 20B-39B, wherein the first group of peptide structures in Table 3A is used to distinguish between the ovarian cancer disease state having the malignant pelvic tumor and a non-ovarian cancer state having a benign pelvic tumor.

41B. The method of any one of embodiments 20B-40B, wherein the peptide structure data comprises quantification data selected from the group consisting of an abundance, a relative abundance, a normalized abundance, a relative quantity, an adjusted quantity, a normalized quantity, a relative concentration, an adjusted concentration, or a normalized concentration.

42B. A method of treating ovarian cancer in a subject comprising receiving peptide structure data corresponding to a biological sample obtained from the subject;
analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 1A, Table 2A, and/or Table 3A; and generating a diagnosis output based on the disease indicator.

43B. The method of embodiment 42B, wherein the disease indicator is based on at least three peptide structures from one of a group of peptide structures identified in Table 3A.

44B. The method of any one of embodiments 42B-43B, further providing a treatment recommendation based upon the diagnosis.

45B. The method of any one of embodiments 42B-44B, further comprising administering a treatment for ovarian cancer.

46B. The method of any one of embodiments 1B-45B, wherein the peptide structure data is generated using multiple reaction monitoring mass spectrometry (MRM-MS).

47B. The method of any one of embodiments 1B-46B, further comprising:
preparing a sample of the biological sample using reduction, alkylation, and enzymatic digestion to form a prepared sample that includes a set of peptide structures.

48B. The method of embodiment 47B, further comprising:
generating the peptide structure data from the prepared sample using multiple reaction monitoring mass spectrometry (MRM-MS).

49B. The method of any one of embodiments 1B-13B and 20B-48B, wherein generating the diagnosis output comprises:
generating a report identifying that the biological sample evidences the ovarian cancer disease state.

50B. The method of embodiment 49B, wherein the treatment output comprises at least one of an identification of a treatment to treat the subject or a treatment plan.

51B. The method of embodiment 50B, further comprising administering the identified treatment or treatment plan to the subject.

52B. The method of any one of embodiments 42B-51B, wherein the treatment comprises at least one of surgery, radiation therapy, a targeted drug therapy, chemotherapy, immunotherapy, hormone therapy, or neoadjuvant therapy.

53B. The method of any one of embodiments 1B-13B and 20B-52B, further comprising:
performing a biopsy of the subject in response to the diagnosis output indicating a positive diagnosis for the ovarian cancer disease state.

54B. The method of any one of embodiments 1B-13B and 20B-53B, further comprising:
generating a report recommending that a biopsy be performed for the subject in response to the diagnosis output indicating a positive diagnosis for the ovarian cancer disease state.

55B. The method of any one of embodiments 1B-13B and 20B-54B, further comprising:
performing a biopsy of the subject in response to the diagnosis output indicating a positive diagnosis for the ovarian cancer disease state.

56B. A method of training a model to diagnose a subject with respect to an ovarian cancer disease state having a malignant pelvic tumor, the method comprising
receiving quantification data for a panel of peptide structures for a plurality of samples for a plurality of subjects,
   wherein the plurality of subjects includes a first portion diagnosed with a negative diagnosis of an ovarian cancer disease state and a second portion diagnosed with a positive diagnosis of the ovarian cancer disease state;
   wherein the quantification data comprises a plurality of peptide structure profiles for the plurality of subjects; and
training a machine learning model using the quantification data to diagnose a biological sample with respect to the ovarian cancer disease state using a group of peptide structures associated with the ovarian cancer disease state,
   wherein the group of peptide structures is identified in Table 3A and listed in Table 3A with respect to relative significance to diagnosing the biological sample.

57B. The method of embodiment 56B, wherein the machine learning model comprises a logistic regression model, optionally a LASSO regression model.

58B. The method of any one of embodiments 56B-57B, further comprising:
identifying an initial plurality of peptide structure profiles;
filtering the initial plurality of peptide structure profiles by a coefficient of variation to generate a plurality of peptide structure profiles for use in training the machine learning model.

59B. The method of embodiment 58B, wherein the filtering is performed to exclude peptide structure profiles having the coefficient of variation at or above 20%.

60B. The method of embodiment 57B, wherein training the machine learning model comprises reducing the plurality of peptide structure profiles using LASSO regression to identify a final group of peptide structures identified in Table 3A.

61B. The method of any one of embodiments 1B-60B, wherein a negative diagnosis for the ovarian cancer disease state indicates a non-ovarian cancer state comprising a benign tumor state.

62B. The method of any one of embodiments 56B-61B, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

63B. The method of any one of embodiments 56B-62B, wherein the trained model uses a relative abundance for a first portion of the first group of peptide structures and a concentration for a second portion of the second group of peptide structures.

64B. The method of any one of embodiments 56B-63B, wherein the training comprises:

identifying a first portion of the plurality of biological samples for subjects with benign pelvic tumors and malignant pelvic tumors and a second portion of the plurality of biological samples for subjects with a healthy status; and
generating a training set of peptide structure profiles for 80% of the first portion and a test set of peptide structure profiles for a remaining 20% of the first portion and the second portion.

65B. The method of any one of embodiments 56B-64B, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

66B. The method of any one of embodiments 56B-65B, further comprising:
generating, using the trained machine learning model, a disease indicator for diagnosing the biological sample with respect to the ovarian cancer disease state; and
generating a report recommending that a biopsy be performed for the subject in response to the diagnosis indicator indicating a positive diagnosis for the ovarian cancer disease state.

67B. The method of any one of embodiments 56B-66B, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

68B. The method of any one of embodiments 56B-66B, further comprising:
generating, using the trained machine learning model, a disease indicator for diagnosing the biological sample with respect to the ovarian cancer disease state; and
   generating a report recommending that a biopsy be performed for the subject in response to the diagnosis indicator indicating a positive diagnosis for the ovarian cancer disease state.

69B. The method of any one of embodiments 1B-68B, wherein the ovarian cancer disease state comprises a malignant pelvic tumor.

70B. The method of any one of embodiments 1B-69B, wherein the ovarian cancer disease state is epithelial ovarian cancer, or optionally malignant epithelial ovarian cancer.

71B. The method of any one of embodiments 1B-70B, wherein the subject is a human.

72B. A kit comprising at least one of a glycopeptide standard, a buffer, or a set of peptide sequences to carry out the method of any one of embodiments 1B-40B, a peptide sequence of the set of peptide sequences identified by a corresponding one of SEQ ID NOS: 111-119, defined in Table 1A and Table 5A.

73B. A composition comprising at least one of peptide structures PS-1-PS-10 and PS-11-PS-34 from Table 1A and Table 2A.

74B. A composition comprising a glycopeptide structure selected as one from a group consisting of peptide structures PS-1, PS-5, PS-11, PS-15, PS-20, PS-25, PS-28, PS-29, PS-30, PS-31, PS-32, and PS-35 to PS-61 identified in Table 3A, wherein:
the peptide structure comprises:
   an amino acid peptide sequence identified in Table 5A as corresponding to the peptide structure; and
   a glycan structure identified in Table 7A as corresponding to the peptide structure in which the glycan structure is linked to a residue of the amino acid peptide sequence at a corresponding position identified in Table 3A; and
   wherein the glycan structure has a glycan composition.

75B. A kit comprising at least one agent for quantifying at least one peptide structure identified in Table 3A to carry out the method of any one of embodiments 20B-55B.

76B. A kit comprising at least one of a glycopeptide standard, a buffer, or a set of peptide sequences to carry out the method of any one of embodiments 20B-52B, a peptide sequence of the set of peptide sequences identified by a corresponding one of SEQ ID NOS: 111, 114, 115, 131, 132, 133, 134, 137, 138, 140, 142, 144, 145, 146, 153-165 identified in Table 3A.

77B. A system comprising:

one or more data processors; and
a non-transitory computer readable storage medium containing instructions which, when executed on the one or more data processors, cause the one or more data processors to perform part or all of any one of embodiments 1B-13B and 20B-55B.

78B. A computer-program product tangibly embodied in a non-transitory machine-readable storage medium, including instructions configured to cause one or more data processors to perform part or all of any one of embodiments 1B-13B and 20B-55B.

### VIII. EXAMPLES

Chemicals and Reagents. Glycoprotein standards purified from human serum/plasma were purchased from Sigma-Aldrich (St. Louis, Mo.). Sequencing grade trypsin was purchased from Promega (Madison, Wis.). Dithiothreitol (DTT) and iodoacetamide (IAA) were purchased from Sigma-Aldrich (St. Louis, Mo.). Human serum was purchased from Sigma-Aldrich (St. Louis, Mo.).

Sample Preparation. Serum samples and glycoprotein standards were reduced, alkylated and then digested with trypsin in a water bath at 37° C. for 18 hours.

LC-MS/MS Analysis. For quantitative analysis, tryptic digested serum samples were injected into an high performance liquid chromatography (HPLC) system coupled to triple quadrupole (QqQ) mass spectrometer. The separation was conducted on a reverse phase column. Solvents A and B used in the binary gradient were composed of mixtures of water, acetonitrile and formic acid. Typical positive ionization source parameters were utilized after source tuning with vendor supplied standards. The following ranges were evaluated: source spray voltage between 3-5 kV, temperature 250-350° C., and nitrogen sheath gas flow rate 20-40 psi. The scan mode of instrument used was dMRM.

For the glycoproteomic analysis, enriched serum glycopeptides were analyzed with a Q Exactive™ Hybrid Quadrupole-Orbitrap™ Mass spectrometer or an Agilent 6495B Triple Quadrupole LC/MS.

MRM Mass Spectroscopy settings, sample preparation, and reagents are set forth in Li, et al., Site-Specific Glycosylation Quantification of 50 serum Glycoproteins Enhanced by Predictive Glycopeptidomics for Improved Disease Biomarker Discovery, Anal. Chem. 2019, 91, 5433-5445; DOI: 10.1021/acs.analchem.9b00776, the entire contents of which are herein incorporated by reference in its entirety for all purposes.

### Example 1—Identifying Glycopeptide Biomarkers

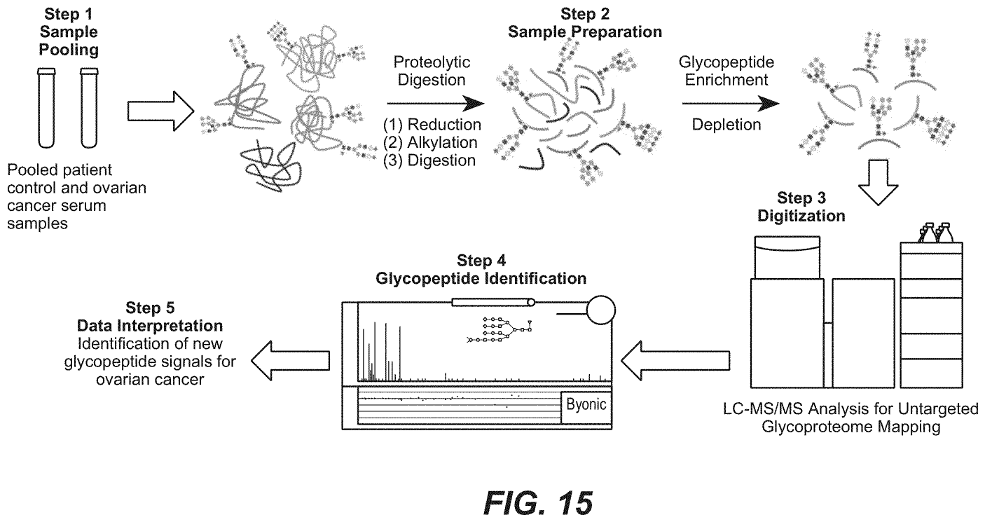

This Example refers to FIGS. 15 and 17-19.

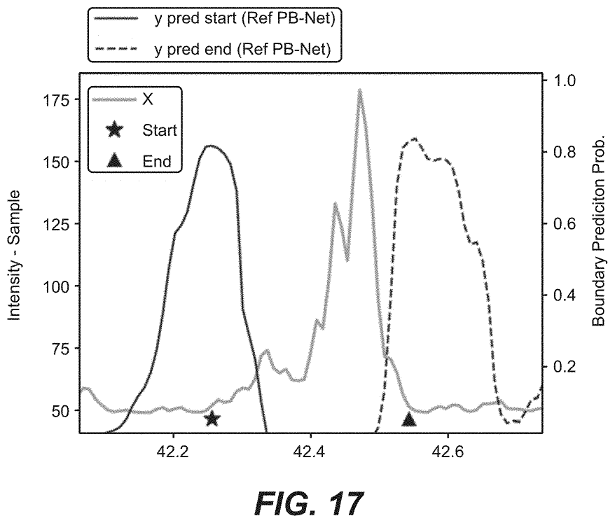

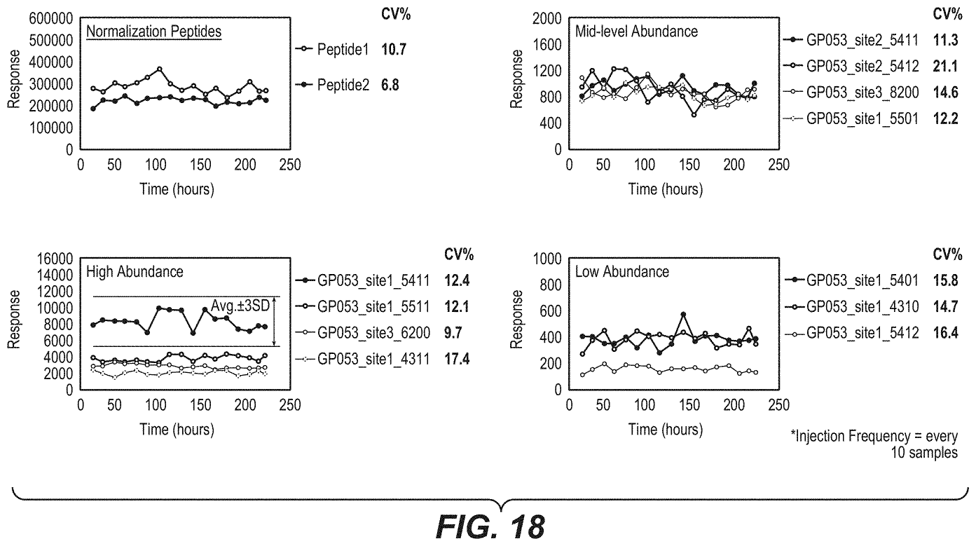

As shown in FIG. 15, in step 1, samples from patients having ovarian cancer and samples from patients not having ovarian cancer were provided. In step 2, the samples were digested using protease enzymes to form glycopeptide fragments. In step 3, the glycopeptide fragments were introduced into a tandem LC-MS/MS instrument to analyze the retention time and MRM-MS transition signals associated with the aforementioned samples. In step 4, glycopeptides and glycan biomarkers were identified. Machine learning systems selected MRM-MS transition signals from a series of MS spectra and associated those signals with the calculated mass of certain glycopeptide fragments. See FIGS. 17-18 for MRM-MS transition signals identified by the machine learning systems.

In step 5, the glycopeptides identified in samples from patients having ovarian cancer were compared using machine learning systems, including lasso regression, with the glycopeptides identified in samples from patients not having ovarian cancer. This comparison included a comparison of the types, absolute amounts, and relative amounts of glycopeptides. From this comparison, normalization of peptides, and relative abundance of glycopeptides was calculated. See FIG. 19 for output results of this comparison.

### Example 2—Identifying Glycopeptide Biomarkers

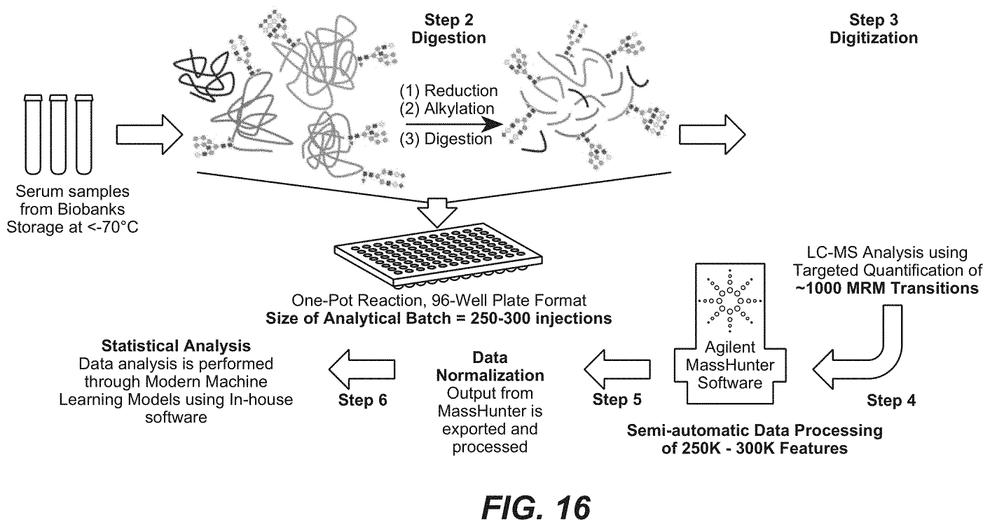

This Example refers to FIG. 16.

As shown in FIG. 16, in step 1, samples from patients were provided. In step 2, the samples are digested in a one pot method using protease enzymes to form glycopeptide fragments. In step 3, the glycopeptide fragments are introduced into a tandem LC-MS/MS instrument to analyze the retention time and MRM-MS transition signals associated with the sample. In step 4, the glycopeptides are identified using machine learning systems which select MRM-MS transition signals and associate those signals with the calculated mass of certain glycopeptide fragments. In step 5, the data is normalized. In step 6, machine learning is used to analyzed the normalized data to identify biomarkers indicative of a patient having ovarian cancer.

### Example 3—Exemplary Retrospective Analysis

### Sample Acquisition

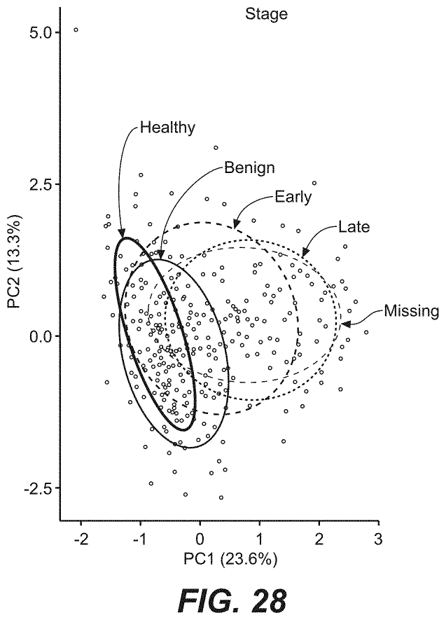

FIG. 26 is a table describing the distribution of the samples acquired in this exemplary retrospective analysis in accordance with one or more embodiments. As shown in FIG. 26, serum samples were acquired from a commercial biobank for 151 women with benign pelvic masses, 145 women with malignant epithelial ovarian cancer (EOC), and 55 healthy controls. Information on stage of EOC was available in 98 of the 145 patients with EOC (see Table 1B). All samples were obtained prior to therapeutic intervention. Information on the benign or malignant nature of tumors was based on histopathological analysis of tissue specimens.

### Sample Processing

Sample processing involved pooled human serum/plasma (e.g., glycoprotein standards purified from human serum/plasma) for assay normalization, dithiothreitol (DTT), and iodoacetamide (IAA), sequencing-grade trypsin, LC-MS-grade water and acetonitrile, and formic acid (LC-MS grade). Serum samples were treated with DTT and IAA to reduce disulfide bonds and to inhibit cysteine proteases, respectively, followed by digestion with trypsin at 37° C. for 18 hours. The digestion was quenched by adding formic acid to each sample to a final concentration of 1% (v/v).

LC-MS analysis included separating digested serum samples over an Agilent ZORBAX Eclipse Plus C18 column (2.1 mm×150 mm i.d., 1.8 μm particle size) using an Agilent 1290 Infinity UHPLC system. The mobile phase A consisted of 3% acetonitrile, 0.1% formic acid in water (v/v), and the mobile phase B of 90% acetonitrile 0.1% formic acid in water (v/v), with the flow rate set at 0.5 mL/minute. The binary solvent composition was set at 100% mobile phase A at the beginning of the run, linearly shifting to 20% B at 20 minutes, 30% B at 40 minutes, and 44% B at 47 minutes. The column was flushed with 100% B and equilibrated with 100% A for a total run time of 70 minutes. After electrospray ionization, operated in positive ion mode, samples were injected into an Agilent 6495B triple quadrupole MS operated in dynamic multiple reaction monitoring (dMRM) mode. The MRM transitions comprised 513 glycopeptide structures which were normalized by comparing them with the abundance of 71 non-glycosylated peptide structures, representing each of 71 proteins from which the glycopeptides monitored were derived. Samples were injected randomized as to underlying phenotype, and reference pooled serum digests were injected interspersed with study samples.

### Data Analysis

Analysis resulted in 683 peptide structures (both peptide and glycopeptide isoforms) being reflected by 1106 MRM transitions, representing 71 high-abundance (concentrations of 10 μg/ml) serum glycoproteins. Our transition list consisted of glycopeptides and non-glycosylated peptides from each glycoprotein. A spectrogram feature recognition and integration software based on recurrent neural networks was used to integrate chromatogram peaks and to obtain molecular abundance quantification for each peptide structure.

Normalized abundances of peptide structures, corrected for within-run drift, were assessed in samples from healthy controls, patients with benign pelvic tumors and those with EOC. Raw abundances were normalized by using spiked-in heavy-isotope-labeled internal standards with known peptide concentrations. The calculation relies either on relative abundance or on site occupancy, i.e., on the fractional abundance across all glycans observed at that site. Log-transformed concentration-normalized data for 501 glycopeptide structures (452 of which are based on on-site occupancy and 49 on relative abundance) and for 70 aglycosylated peptide structures were ultimately used for the analysis, totaling 571 unique peptide structures. Fold changes for individual peptide structures were calculated on normalized abundances of healthy (control) vs. EOC samples and benign tumor vs. EOC samples. False discovery rates (FDR) were calculated using the Benjamini-Hochberg method. Principal component analysis (PCA) was performed on log-concentration-normalized abundances of glycopeptide structures to investigate differences among the three phenotypes (e.g., healthy control, EOC, and benign pelvic tumor) studied. Prior to performing PCA, normalized abundances were scaled such that the distributions of all biomarkers were Gaussian with zero mean and unit variance.

To compare any two phenotypes, age-adjusted linear regression was used on a feature-by-feature basis with phenotype serving as the sole binary independent variable. Correcting for multiple comparisons, differences of any biomarker among phenotype groups compared were considered statistically significant where the FDR was less than 0.05. Examples of features include relative abundance (or normalized relative abundance), concentration (or normalized concentration), and site occupancy (fractional abundance across all glycans observed at the corresponding linking site of the corresponding peptide sequence).

For supervised multivariate modeling, a total of 1084 features (571 concentration, 49 relative abundance, and 464 site occupancy features) were log-transformed and split into a training set formed by 80% of all samples from women with benign pelvic tumors and EOC, and a testing set formed by the remaining 20% of these women and all healthy controls. To perform binary classification and predict the probability of EOC, repeated five-fold cross-validated LASSO-regularized logistic regression was used with hyperparameters tuned to prevent overfitting and promote balanced sensitivity and specificity metrics. Training of the binary classification model was performed using the subset of the 1084 total features having low coefficients of variation (<20%) in pooled serum replicates. This subset included 976 features, with each feature being a concentration, relative abundance, or site occupancy for a corresponding peptide structure and where some peptide structures correspond with multiple features. For example, a given peptide structure may be associated with one, two, or three features within the subset of the 976 features.

### Results

Normalized abundances of 428 peptide structures were found to display statistically significantly different abundances (FDR<0.05) in samples of patients with benign pelvic tumors and samples of patients with EOC. 139 peptide structures had statistically significant abundance differences between benign vs. early stage (e.g., stage 1 or 2) EOC. 412 peptide structures had statistically significant abundance differences between benign vs. late stage (e.g., stage 3 or 4) EOC, 137 of which overlapped with those for benign v. early stage. When comparing samples of healthy controls with samples from all EOCs, benign tumors, early stage (e.g., stage 1 or 2) EOC, and late stage (e.g., stage 3 or 4) EOC, statistically significant abundances were found for 386, 149, 215, and 365 markers, respectively. 120 peptide structures were found to be statistically significantly differentially abundant in healthy controls vs. patients with benign pelvic tumors, and in healthy control vs. EOC. 200 peptide structures were found to be statistically significantly differentially abundant in in healthy control vs. early stage EOC and healthy control vs. late stage EOC. Lastly, of the 428 and 386 markers that were found statistically significantly differentially expressed between EOC vs. benign pelvic tumors and EOC vs. healthy controls, respectively, 328 were shared.

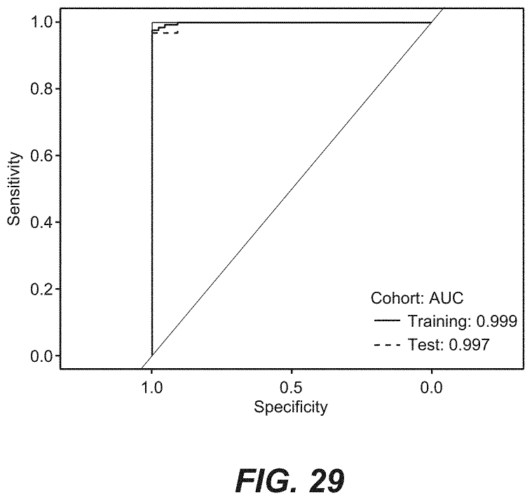

FIG. 27 is a plot diagram illustrating the results of a principal component analysis performed to assess the segregation between healthy, benign pelvic tumor, and EOC samples across first and second principal components in accordance with one or more embodiments. Generally, EOC samples segregated distinctly from healthy control samples, while most benign pelvic tumors did not segregate as distinctly from healthy control samples.

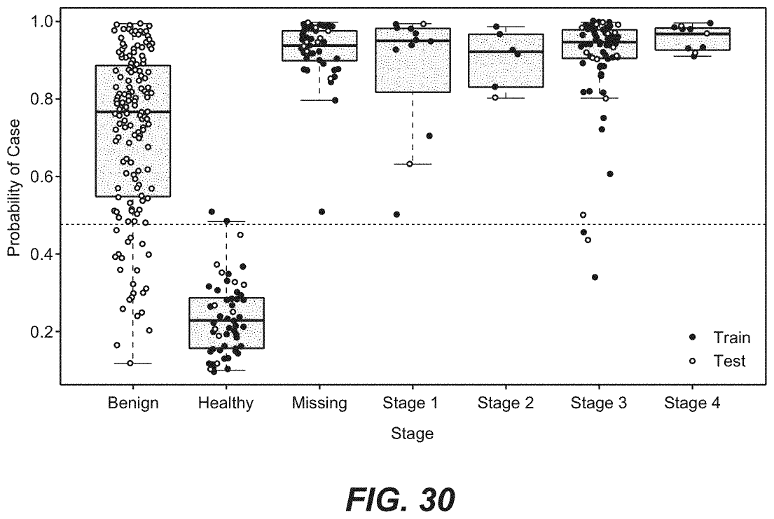

FIG. 28 is a plot diagram illustrating the results of a principal component analysis performed to assess segregation between healthy, benign pelvic tumor, early EOC, late EOC, and missing (undocumented) samples). Generally, EOC samples (and in particular late stage EOC samples) segregated distinctly from healthy control samples, while most benign pelvic tumors did not segregate as distinctly from healthy control samples.

### Results in Context of Screening for Malignant EOC

To assess the suitability of serum glycoproteomics in the context of screening for malignant EOC, a multivariable model was built to predict EOC vs. healthy status. This multivariable model is a supervised machine learning model that includes a logistic regression model, the logistic regression model including a LASSO regression model. Repeated cross-validation in the training set established the optimal LASSO hyperparameter (lambda=0.0608, cross-validated F1=0.971). Applying this amount of shrinkage to the panel of 976 features resulted in a logistic model with 10 peptide structures with non-zero coefficients.

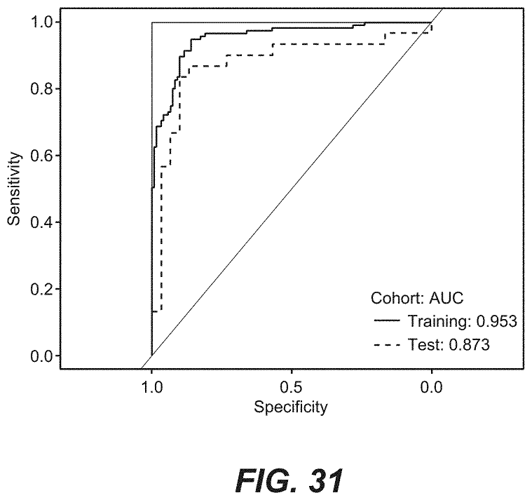

FIG. 29 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.975, sensitivity=0.983, specificity=0.955) and the test set (accuracy=0.976, sensitivity=0.967, specificity=1.0). Further, ROC analysis demonstrated strong performance across a range of cutoffs, and little overfitting, with the training AUC (area under the curve)=0.999 and test AUC=0.997.

Thus, the multivariable model that was built may be used accurately and reliably to malignant EOC and distinguish such malignancy from a healthy status. Such diagnostic power may be used to reduce the need for unnecessary invasive testing. Further, such diagnostic information can be used to identify patients with EOC earlier, which may lead to earlier treatment, improved treatment recommendations, and improved treatment plans.

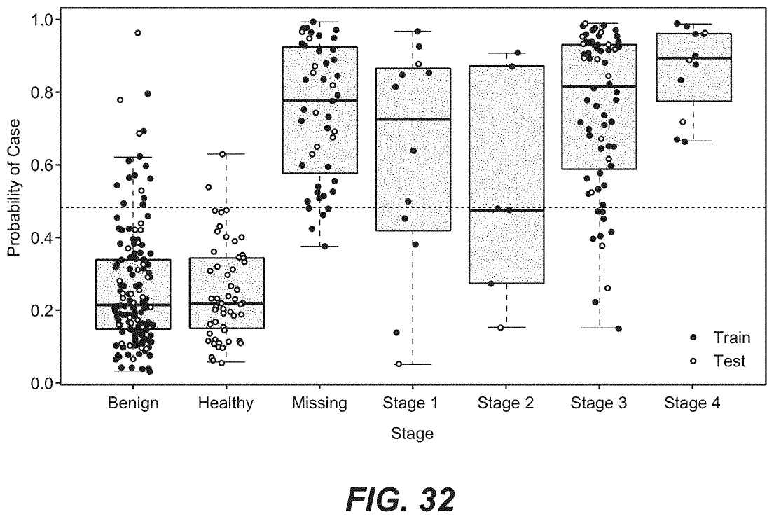

FIG. 30 is an illustration of a diagram showing the probability distributions for the various groups using the multivariable model for predicting malignancy v. benign status of pelvic tumors in accordance with one or more embodiments. As shown in FIG. 30, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the solidly predicts malignancy and severity of disease.

Table 8A below provides the fold changes, FDRs, and p-values for the 10 peptide structures PS-1 to PS-10 (same as those in Table 1A above) based on differential expression analysis (DEA). The peptide structures PS-1 to PS-10 are ordered both in Table 1A and in Table 8A with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 8A
Peptide Structure Markers for Regression Model to distinguish between
Epithelial Ovarian Cancer and Healthy State

| | | Healthy v | | | |
|---|---|---|---|---|---|
| PS- | | EOC | Healthy v | Healthy v | |
| ID | | (Fold | EOC | EOC | |
| NO. | PS-NAME | Change) | (FDR) | (p-value) | Feature |
| PS-1 | ZA2G_128_5402 | 1.57212 | 1.99E−13 | 3.14E−15 | relab |
| PS-2 | IC1_253_6503 | 2.26917 | 6.42E−18 | 2.25E−20 | conc |
| PS-3 | CFAI_494_5402 | 1.30391 | 3.00E−07 | 4.78E−08 | relab |
| PS-4 | CERU_138_6513 | 1.37235 | 2.14E−06 | 4.85E−07 | relab |
| PS-5 | IGG1_297_3410 | 1.98807 | 1.03E−09 | 6.47E−11 | conc |
| PS-6 | HEMO_64_5402 | 1.53316 | 3.06E−11 | 1.12E−12 | relab |
| PS-7 | APOB_983_5402 | 1.98566 | 1.11E−13 | 1.17E−15 | conc |
| CK-1 | FINC_SYTITGL_ | 0.51932 | 9.92e−09 | 1.043e−09 | relab |
| | QPGTDYK | | | | |
| PS-8 | HPT_207_121005 | 2.21826 | 3.17E−10 | 1.66E−11 | conc |
| PS-9 | IGG3_297_3400 | N/A | N/A | N/A | relab |
| PS-10 | IGG4_297_3400 | N/A | N/A | N/A | relab |
| CK-2 | APOM_135_ | 0.59098 | 1.58e−17 | 8.28e−20 | conc |
| | 8500_CHK | | | | |

### Results in Context of Triaging Pelvic Tumors

To assess the suitability of serum glycoproteomics in the context of clinically triaging pelvic tumors, a multivariable model was built to predict malignancy vs. benign status of such pelvic tumors. This multivariable model is a supervised machine learning model that includes a logistic regression model, the logistic regression model including a LASSO regression model. Repeated cross-validation in the training set established the optimal LASSO hyperparameter (lambda=0.045, cross-validated F1=0.849). Applying this amount of shrinkage to the panel of 976 features resulted in a logistic model with 25 peptide structures with non-zero coefficients.

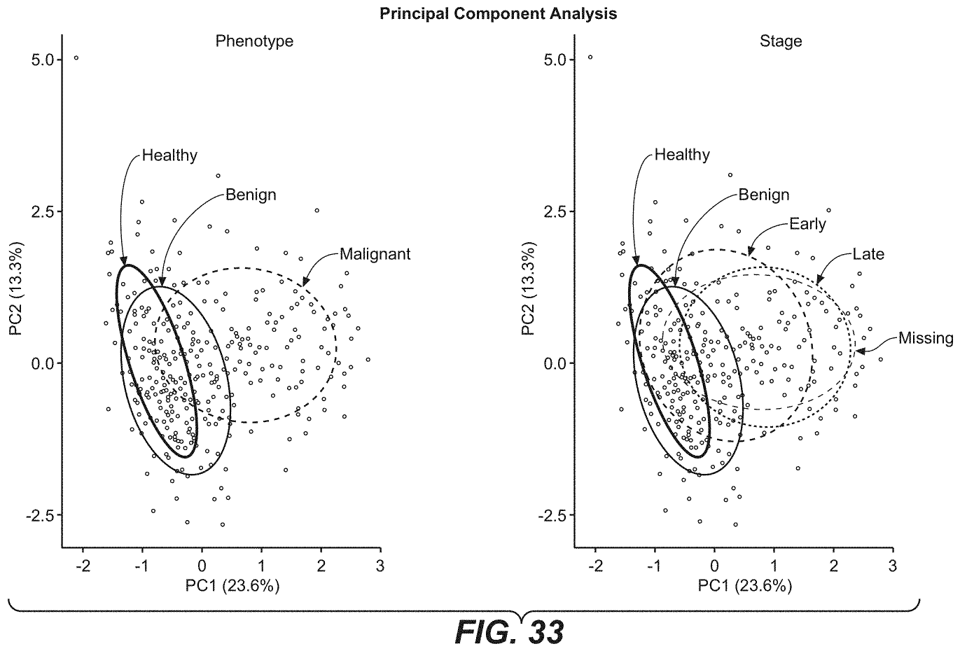

FIG. 31 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.869, sensitivity=0.835, specificity=0.901) and the test set (accuracy=0.867, sensitivity=0.867, specificity=0.867). Further, ROC analysis demonstrated strong performance across a range of cutoffs, and little overfitting, with the training AUC (area under the curve)=0.953 and test AUC=0.873.

Thus, the multivariable model that was built may be used accurately and reliably to triage pelvic tumors and distinguish those that are malignant from those that are benign. Such diagnostic power may be used to reduce the need for invasive testing (e.g., biopsy) prior to treatment can be administered. Further, such diagnostic information can be used to improve treatment recommendations and treatment plans (e.g., earlier treatment in the case of malignant EOC) and reduce indications for unnecessary treatment (e.g., no indication for surgery when the pelvic tumor is benign).

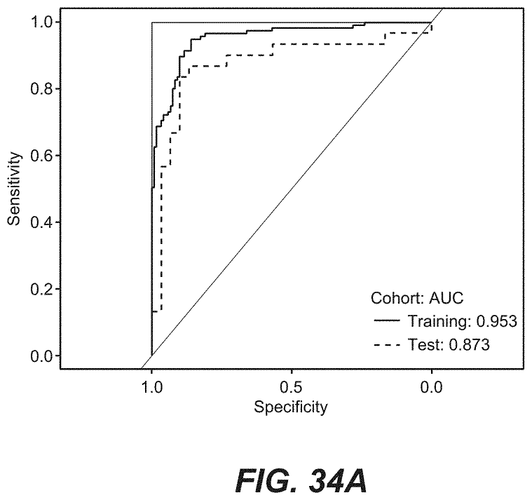

FIG. 32 is an illustration of a diagram showing the probability distributions for the various groups using the multivariable model for predicting malignancy v. benign status of pelvic tumors in accordance with one or more embodiments. As shown in FIG. 30, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the 25 peptide structures for the LASSO regression model solidly predict malignancy and severity of disease.

Table 9A below provides the fold changes, FDRs, and p-values for the 25 peptide structures PS-5 and PS-11 to PS-34 (same as those in Table 2A above) based on differential expression analysis (DEA). The peptide structures PS-5 and PS-11 to PS-34 are ordered both in Table 2A and in Table 9A with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 9A
Peptide Structure Markers for Regression Model to distinguish between
Epithelial Ovarian Cancer and Benign Pelvic Tumor

| | | Benign v. | | | |
|---|---|---|---|---|---|
| | | EOC | Benign v. | Benign v. | |
| PS-ID | | (Fold | EOC | EOC | |
| NO. | PS-NAME | Change) | (FDR) | (p-value) | Feature |
| CK-3 | APOD_98_ | 1.54848 | 4.78e−13 | 8.46e−14 | relab |
| | 9800_CHECK | | | | |
| PS-11 | CO2_621_ | 1.36880 | 1.73E−11 | 3.66E−12 | relab |
| | 5200 | | | | |
| PS-5 | IGG1_297_ | 1.54336 | 2.47E−10 | 6.61E−11 | relab |
| | 3410 | | | | |
| PS-12 | AGP1_93_ | 2.39546 | 2.79E−16 | 2.20E−17 | relab |
| | 7612 | | | | |
| PS-13 | AACT_271_ | 1.68006 | 2.27E−08 | 7.70E−09 | conc |
| | 7602 | | | | |
| PS-14 | A2MG_1424_ | 1.15594 | 0.007733584 | 0.005106062 | relab |
| | 5402 | | | | |
| PS-15 | AACT_271_ | 2.34075 | 2.81E−18 | 1.04E−19 | relab |
| | 6513 | | | | |
| PS-16 | CERU_397_ | 1.07300 | 0.008195667 | 0.005425503 | relab |
| | 5402 | | | | |
| PS-17 | APOB_3411_ | 1.018081 | 0.743228938 | 0.714593147 | relab |
| | 5301 | | | | |
| PS-18 | AACT_106_ | 2.11211 | 1.42E−16 | 9.67E−18 | relab |
| | 6513 | | | | |
| PS-19 | CERU_138_ | 1.08927 | 0.002831028 | 0.001760096 | conc |
| | 5402 | | | | |
| PS-20 | A1AT_107_ | 2.15635 | 6.82E−14 | 1.06E−14 | relab |
| | 6513 | | | | |
| PS-21 | AGP1_93_ | 1.11780 | 0.012740002 | 0.008679266 | relab |
| | 7602 | | | | |
| PS-22 | VTNC_242_ | 0.83257 | 0.000446981 | 0.000252845 | relab |
| | 6502 | | | | |
| PS-23 | IGG2_297_ | 0.69463 | 8.28E−10 | 2.36E−10 | conc |
| | 3510 | | | | |
| PS-24 | CFAH_882_ | 0.84102 | 1.06E−05 | 4.78E−06 | relab |
| | 5411 | | | | |
| CK-4 | APOM_135_ | 0.81884 | 1.16e−08 | 3.87e−09 | conc |
| | 8500_CHECK | | | | |
| PS-25 | AGP1_103_ | 1.18615 | 0.001152856 | 0.000676369 | relab |
| | 8704 | | | | |
| PS-26 | IGG1_297_ | 0.60088 | 2.09E−11 | 4.47E−12 | relab |
| | 4300 | | | | |
| PS-27 | APOH_253_ | 0.62217 | 1.65E−16 | 1.16E−17 | conc |
| | 5401 | | | | |
| PS-28 | APOD_98_ | 0.71180 | 1.50E−12 | 2.82E−13 | conc |
| | 5411 | | | | |
| PS-29 | TRFE_630_ | 0.69298 | 4.01E−14 | 5.62E−15 | conc |
| | 5411 | | | | |
| PS-30 | CERU_138_ | 0.81476 | 7.13E−07 | 2.87E−07 | relab |
| | 6502 | | | | |
| PS-31 | A2MG_1424_ | 0.67638 | 1.53E−23 | 2.68E−26 | conc |
| | 5411 | | | | |
| PS-32 | A2MG_55_ | 0.71212 | 2.20E−20 | 1.93E−22 | conc |
| | 5411 | | | | |
| PS-33 | TRFE_630_ | 0.77453 | 1.01E−09 | 2.95E−10 | conc |
| | 5412 | | | | |
| PS-34 | IGG2_297_ | 0.73039 | 3.50E−08 | 1.23E−08 | conc |
| | 4511 | | | | |

### Molecular Pathway Analysis

### Ingenuity Pathway Analysis (IPA)

Of 59 proteins for which informative glycopeptide abundance differences were found among the phenotype contrasts evaluated, 55 were successfully mapped to accessions in the IPA knowledge base. Among these, and after filtering against an FDR of <0.05, 47, 39, and 41 features were found to be statistically significantly discordant in late-stage disease vs. healthy, early-stage disease vs. healthy, and benign disease vs. healthy phenotype contrasts, respectively.

### IPA: Canonical Pathways Enrichment

Of the 73, 67, and 78 canonical pathways reported to be enriched by IPA, 27, 20 and 27 were found to reach statistical significance (p-value≤0.05) in late-stage disease vs. healthy, early-stage disease vs. healthy and benign disease vs. healthy study comparisons, respectively, with 19 pathways found to be shared among all three contrasts, including LXR/RXR activation, FXR/RXR activation, acute phase response signaling, and the coagulation system, among others (Table 2B).

Substantial overlap was observed between members of the LXR/RXR activation and the FXR/RXR activation pathways (Table 2B). Similarly, overlap was seen among members of the “atherosclerosis signaling, glycoform-mediated endocytosis signaling”, “IL-12 signaling and production in macrophages”, and the “production of nitric oxide and reactive oxygen species in macrophages” pathways. These include predominantly the apolipoproteins, APOB, APOC3, APOD, APOE, and APOM, as well as CLU, ORM1, and SERPINAL A role for immune modulation was suggested by the observed enrichment of the “primary immunodeficiency syndrome” canonical pathway. Members of the pathway from the data set include the IGHA1, IGHG1, IGHG2 and IGHM gene products. Likewise, the “coagulation system” canonical pathway, involving the A2M, KNG1, and SERPINA1 gene products, was found to be associated with the findings described herein.

### IPA: Upstream Regulators

IPA identified 208, 194, and 201 potential upstream regulators associated with differentially expressed protein features in the benign disease vs. healthy, the early-stage disease vs. healthy, and the late-stage disease vs. healthy comparisons, respectively, at p≤0.05. Potential upstream regulators that were common across study comparisons include a broad range of factors. With a mean p-value estimate of 8.6e-11, the hepatocyte nuclear factor 1-alpha (HNF1A), a transcription factor, topped the list of significant upstream regulators across study comparisons. Its target molecules in our study data include the AHSG, APOH, APOM, C1S, C4BPA, ITIH4, SERPINA1, SERPING1, and YIN gene products. The proinflammatory cytokine molecule, interleukin 6 (IL6), ranked next (mean p-value=8.8e-08). Its targets include the AGT, APOB, CLU, HP, ORM1, SERPINA1, SERPINA3 gene products in our dataset. Rounding out the top 10 most significant upstream regulators were HNF4A, SREBF1, PPARA, RXRA, NR1H3, IL22, TCF and SMARCA4.

### Reactome Pathway Analysis (RPA): Differentially Expressed Features

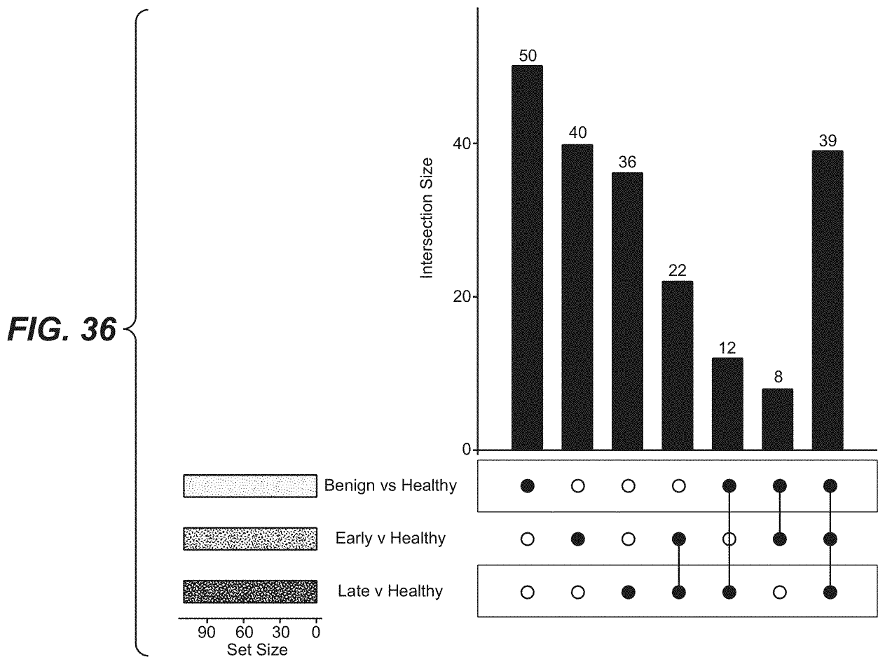

Ranking by p-values for differential abundance of peptide/glycopeptide features, the top 10 percentile statistically most significant features were selected from the benign disease vs. healthy, early-stage disease vs. healthy, and late-stage disease vs. healthy study comparisons. 50, 40, and 36 features were found to be differentially abundant respectively (FIG. 36). Considering only glycopeptide features quantified by relative site occupancy measures, 13 were found in common across our study contrasts These glycopeptides mapped to protein product of the genes APOM, SERPING1, CFI, A2M, SLC25A6, AZGP1, FN1 and LRG1. Five of these significant and consistent differentially expressed glycopeptides are associated with the C1-inhibitor protein, a product of the SERPING1 gene. These glycopeptides include the sialylated series IC1-253-6503, IC1-238-5402, IC1-352-5402, IC1-352-5412, IC1-253-5412.

### RPA Enrichment

Filtering at the p-value estimate of ≤0.05, RPA enrichment analysis identified eight significantly enriched pathways. These include the platelet degranulation, response to elevated platelet cytosolic Ca2+, intrinsic pathway of fibrin clot formation, formation of fibrin clot (clotting cascade), regulation of complement cascade, platelet activation, signaling and aggregation, complement cascade and the degradation of the extracellular matrix pathways—associated with the SERPING1, A2M, CFI and FN1 gene products.

### STRING Analysis

Comparing estimated enriched pathways based on IPA and RPA supports a true enrichment of the acute phase response signaling and complement system canonical pathways, with the SERPING1, A2M, FN1 and/or CFI molecules shared. The STRING database (v11.5) was searched for documented and inferred relationships among elements of the significantly enriched functional pathways from both IPA and RPA. These included elements of the complement system and the acute phase response signaling canonical pathways. Consisting of 23 unique nodes, 154 edges were found. A highly connected network was observed—the average node degree was 13.4 and average local clustering coefficient was 0.709. Against an expected number of edges of 4, the protein-protein-interaction enrichment p-value was <1.0e-16.

### Example 4—Exemplary Retrospective & Prospective Analysis

A validation study was conducted using both retrospective patient samples and samples collected prospectively in the ongoing Clinical Validation of the InterVenn Ovarian CAncer Liquid Biopsy (VOCAL) study. Samples included those from patients with malignant EOC and patients with benign pelvic tumors. Samples were processed in a manner similar to the manner described for the Exemplary Retrospective Analysis in Section VII.A above.

A logistic regression model was built identifying a panel of 38 peptide structures (same as those in Table 3A above). This panel of 38 peptide structures had an overall predictive accuracy of over 86% for the prediction of malignancy versus benign status of pelvic tumors.

Table 10A provides the fold changes and p-values for the 38 peptide structures also identified in Table 3A above based on differential expression analysis (DEA). These peptide structures are ordered both in Table 3A and in Table 10A with respect to relative significance to the probability score generated by the model based on p-values. In this context, more significant peptide structures have lower p-values, while less significant peptide structures have higher p-values. In other words, relative significance to the probability score decreased with increasing p-values.

TABLE 10A

| PS-ID | Peptide Structure (PS) | Fold | |
|---|---|---|---|
| NO. | NAME | change | P value |
| PS-35 | VTNC_169_5401 | 0.673832581 | 7.71E−28 |
| PS-36 | FETUA_176_6513 | 1.773640576 | 4.75E−26 |
| PS-37 | AGP1_93_7614 | 2.422571074 | 6.31E−25 |
| PS-38 | QUANTPEP.A2GL_ | 1.801062322 | 1.02E−24 |
| | DLLLPQPDLR | | |
| PS-39 | HPT_184_5402 | 1.953879772 | 3.07E−22 |
| PS-40 | TRFE_432_6503 | 1.348502947 | 1.44E−21 |
| PS-41 | TRFE_630_6513 | 1.515265874 | 2.57E−20 |
| PS-42 | HEMO_453_5402 | 1.04945304 | 4.16E−20 |
| PS-43 | QUANTPEP.TTR_ | 0.703228829 | 7.47E−20 |
| | TSESGELHGLT_ | | |
| | TEEEFVEGIYK | | |
| PS-5 | IGG1_297_3410 | 1.367775892 | 1.35E−19 |
| PS-44 | TRFE_630_5400 | 1.664954512 | 1.53E−19 |
| PS-45 | AGP1_103_9804 | 0.653523308 | 2.49E−19 |
| PS-46 | TRFE_432_6501 | 0.727398423 | 6.64E−19 |
| PS-47 | HPT_241_5402 | 1.73178282 | 1.34E−18 |
| PS-48 | IGG1_297_5510 | 0.667010971 | 1.79E−18 |
| PS-49 | QUANTPEP.AFAM_ | 0.760270627 | 9.26E−18 |
| | SDVGFLPPFPTLDPEEK | | |
| PS-32 | A2MG_55_5411 | 0.806560345 | 5.66E−17 |
| PS-50 | IGG2_297_5510 | 0.585930965 | 6.42E−17 |
| PS-51 | AGP1_103_7603 | 0.72406383 | 8.10E−17 |
| PS-52 | IGG2_297_5400 | 0.596169156 | 1.99E−15 |
| PS-1 | ZA2G_128_5402 | 1.226010701 | 6.99E−15 |
| PS-53 | TRFE_630_6502 | 0.793580625 | 1.14E−14 |
| PS-54 | TRFE_432_6502 | 0.807605258 | 1.24E−14 |
| PS-55 | IGG2_297_4510 | 0.675549742 | 1.56E−14 |
| PS-56 | AACT_106_7614 | 1.624200983 | 2.36E−14 |
| PS-57 | PEP-APOA1_ | 0.814761281 | 7.40E−14 |
| | VSFLSALEEYTK | | |
| PS-11 | CO2_621_5200 | 1.158420675 | 8.05E−14 |
| PS-15 | AACT_271_6513 | 1.421889994 | 6.46E−13 |
| PS-58 | FETUA_176_5401 | 0.749741527 | 7.94E−13 |
| PS-59 | FETUA_346_1102 | 0.790597963 | 1.04E−12 |
| PS-60 | PEP-APOA1_ | 0.835672133 | 4.10E−12 |
| | THLAPYSDELR | | |
| PS-29 | TRFE_630_5411 | 0.789766926 | 6.37E−12 |
| PS-25 | AGP1_103_8704 | 0.828638044 | 1.43E−11 |
| PS-30 | CERU_138_6502 | 0.767515416 | 8.99E−11 |
| PS-20 | A1AT_107_6513 | 1.459459075 | 1.13E−10 |
| PS-31 | A2MG_1424_5411 | 0.868832513 | 4.00E−08 |
| PS-28 | APOD_98_5411 | 0.970828127 | 0.069865069 |
| PS-61 | C4BPA_221_5402 | 1.010407554 | 0.120929566 |

TABLE 1
IX. TABLES
Transition Numbers for Glycopeptides from Glycopeptide Groups

| Transition | | |
|---|---|---|
| No. | Compound Group | Compound Name |
| 1 | P01009\|Alpha-1- | A1AT-GP001_107_6513 |
| | antitrypsin\|A1AT | |
| 2 | P01023\|Alpha-2- | A2MG-GP004_ |
| | macroglobulin\|A2MG | 1424_5411 |
| 3 | P01023\|Alpha-2- | A2MG-GP004_55_5411 |
| | macroglobulin\|A2MG | |
| 4 | P01011\|Alpha-1- | AACT-GP005_106_7614 |
| | antichymotrypsin\|AACT | |
| 5 | P01011\|Alpha-1- | AACT-GP005_271_6513 |
| | antichymotrypsin\|AACT | |
| 6 | P02763\|Alpha-1-acid | AGP1-GP007_103_7603 |
| | glycoprotein 1\|AGP1 | |
| 7 | P02763\|Alpha-1-acid | AGP1-GP007_103_8704 |
| | glycoprotein 1\|AGP1 | |
| 8 | P02763\|Alpha-1-acid | AGP1-GP007_103_9804 |
| | glycoprotein 1\|AGP1 | |
| 9 | P02763\|Alpha-1-acid | AGP1-GP007_93_7614 |
| | glycoprotein 1\|AGP1 | |
| 10 | P05090\|Apolipoprotein | APOD-GP014_98_5411 |
| | D\|APOD | |
| 11 | P05090\|Apolipoprotein | APOD-GP014_98_9800 |
| | D\|APOD | |
| 12 | P04003\|C4b-binding | C4BPA-GP076_221_5402 |
| | protein alpha | |
| | chain\|C4BPA | |
| 13 | P00450\|Ceruloplasmin\|CERU | CERU-GP023_138_6521 |
| 14 | P06681\|ComplementC2\|CO2 | CO2_621_5200 |
| 15 | P02765\|Alpha-2-HS- | FETUA-GP036_ |
| | glycoprotein\|FETUA | 176_5401 |
| 16 | P02765\|Alpha-2-HS- | FETUA-GP036_ |
| | glycoprotein\|FETUA | 176_6513 |
| 17 | P02765\|Alpha-2-HS- | FETUA-GP036_ |
| | glycoprotein\|FETUA | 346_1102 |
| 18 | P02790\|Hemopexin\|HEMO | HEMO-GP042_453_ |
| | | 5402/5421 |
| 19 | P01857\|Immunoglobulin heavy | IgG1-GP048_297_3410 |
| | constant gamma 1\|IgG1 | |
| 20 | P01857\|Immunoglobulin heavy | IgG1-GP048_297_5510 |
| | constant gamma 1\|IgG1 | |
| 21 | P01859\|Immunoglobulin heavy | IgG2-GP049_297_4510 |
| | constant gamma 2\|IgG2 | |
| 22 | P01859\|Immunoglobulin heavy | IgG2-GP049_297_5400 |
| | constant gamma 2\|IgG2 | |
| 23 | P01859\|Immunoglobulin heavy | IgG2-GP049_297_5510 |
| | constant gamma 2\|IgG2 | |
| 24 | P27169\|Serum | PON1-GP060_324_6501 |
| | paraoxonase/arylesterase | |
| | 1\|PON1 | |
| 25 | P02750\|Leucine-richAlpha-2- | QuantPep-A2GL-GP003_ |
| | glycoprotein\|A2GL | DLLLPQPDLR |
| 26 | P43652\|Afamin\|AFAM | QuantPep-AFAM- |
| | | GP006_SDVGFL- |
| | | PPFPTLDPEEK |
| 27 | P20807\|Calpain-3\|CAN3 | QuantPep-CAN3-GP022_ |
| | | FIIDGANR |
| 28 | P02766\|Transthyretin\|TTR | QuantPep-TTR- |
| | | GP065_TSESGELHG- |
| | | LTTEEEFVEGIYK |
| 29 | Q9UPW8\|Protein unc- | QuantPep-UN13A- |
| | 13HomologA\|UN13A | GP066_ |
| | | LDLGLTVEVWNK |
| 30 | P02787\|Serotransferrin\|TRFE | TRFE-GP064_432_6501 |
| 31 | P02787\|Serotransferrin\|TRFE | TRFE-GP064_432_6502 |
| 32 | P02787\|Serotransferrin\|TRFE | TRFE-GP064_432_6503 |
| 33 | P02787\|Serotransferrin\|TRFE | TRFE-GP064_630_5400 |
| 34 | P02787\|Serotransferrin\|TRFE | TRFE-GP064_630_5411 |
| 35 | P02787\|Serotransferrin\|TRFE | TRFE-GP064_630_6502 |
| 36 | P02787\|Serotransferrin\|TRFE | TRFE-GP064_630_6513 |
| 37 | P04004\|Vitronectin\|VTNC | VTNC-GP067_169_5401 |
| 38 | P25311\|Zinc-alpha-2- | ZA2G-GP068_128_5402 |
| | glycoprotein\|ZA2G | |

TABLE 2
Transition Numbers with Precursor
Ion and Product Ion (m/z)

| Transition No. | Precursor Ion | Product Ion |
|---|---|---|
| 1 | 1341 | 366.1 |
| 2 | 1057 | 366.1 |
| 3 | 1115.4 | 366.1 |
| 4 | 1214.1 | 274.1 |
| 5 | 1191.2 | 366.1 |
| 6 | 1335 | 366.1 |
| 7 | 1165.6 | 366.1 |
| 8 | 1256.8 | 366.1 |
| 9 | 1116.9 | 366.1 |
| 10 | 1079.7 | 366.1 |
| 11 | 1335.3 | 366.1 |
| 12 | 1116.9 | 366.1 |
| 13 | 1117.2 | 366.1 |
| 14 | 891.1 | 829.4 |
| 15 | 1070.4 | 366.1 |
| 16 | 1343.8 | 366.1 |
| 17 | 988.8 | 274.1 |
| 18 | 1314.9 | 366.1 |
| 19 | 879 | 204.1 |
| 20 | 1054.7 | 366.1 |
| 21 | 989.9 | 204.1 |
| 22 | 927.7 | 366.1 |
| 23 | 1043.8 | 366.1 |
| 24 | 1149.3 | 366.1 |
| 25 | 590.3 | 725.4 |
| 26 | 944.5 | 1269.6 |
| 27 | 453.2 | 532.2 |
| 28 | 819.1 | 855.5 |
| 29 | 693.9 | 675.4 |
| 30 | 1252.5 | 366.1 |
| 31 | 1012.7 | 366.1 |
| 32 | 1085.4 | 366.1 |
| 33 | 1035.6 | 366.1 |
| 34 | 1144.9 | 366.1 |
| 35 | 1018.1 | 366.1 |
| 36 | 1105.6 | 366.1 |
| 37 | 942.4 | 366.1 |
| 38 | 1115.1 | 366.1 |

MS1 and MS2 resolution was 1 unit.

TABLE 3
Transition Numbers with Retention Time, ΔRetention
Time, Fragmentor and Collision Energy

| Transition | Ret Time | Delta Ret | | Collision |
|---|---|---|---|---|
| No. | (min) | Time | Fragmentor | Energy |
| 1 | 43.4 | 1.6 | 380 | 34 |
| 2 | 43.7 | 1.6 | 380 | 22 |
| 3 | 41.7 | 1.4 | 380 | 22 |
| 4 | 38.6 | 1.2 | 380 | 35 |
| 5 | 31.9 | 1.4 | 380 | 30 |
| 6 | 5.8 | 1.6 | 380 | 34 |
| 7 | 5.8 | 1.6 | 380 | 29 |
| 8 | 5.6 | 1.6 | 380 | 25 |
| 9 | 23.9 | 1.4 | 380 | 25 |
| 10 | 24 | 1.4 | 380 | 20 |
| 11 | 31 | 1.4 | 380 | 33 |
| 12 | 37.5 | 1.4 | 380 | 25 |
| 13 | 16.9 | 1.4 | 380 | 34 |
| 14 | | 1.4 | 380 | 20 |
| 15 | 30.4 | 1.4 | 380 | 26 |
| 16 | 31.1 | 1.6 | 380 | 34 |
| 17 | 23 | 2.4 | 380 | 20 |
| 18 | 31.2 | 1.5 | 380 | 30 |
| 19 | 8 | 1.3 | 380 | 21 |
| 20 | 8.1 | 1.3 | 380 | 20 |
| 21 | 13.2 | 1.2 | 380 | 15 |
| 22 | 13.2 | 1.2 | 380 | 25 |
| 23 | 13.1 | 1.2 | 380 | 25 |
| 24 | 34.2 | 1.4 | 380 | 25 |
| 25 | 31.3 | 1.4 | 380 | 15 |
| 26 | 40.3 | 1.4 | 380 | 29 |
| 27 | 15.7 | 1.2 | 380 | 12 |
| 28 | 34.4 | 1.3 | 380 | 25 |
| 29 | 40 | 1.2 | 380 | 20 |
| 30 | 26.4 | 1.4 | 380 | 20 |
| 31 | 27.4 | 1.4 | 380 | 25 |
| 32 | 28 | 1.4 | 380 | 27 |
| 33 | 31 | 1.4 | 380 | 25 |
| 34 | 31.9 | 1.6 | 380 | 30 |
| 35 | 33 | 1.4 | 380 | 25 |
| 36 | 33.8 | 1.4 | 380 | 27 |
| 37 | 24.3 | 1.4 | 380 | 23 |
| 38 | 10.8 | 1.4 | 380 | 30 |

Cell accelerator voltage was 5.

TABLE 4
Glycan Residue Compound Numbers,
Molecular Mass, and Glycan Fragment
mass-to-charge (m/z) (+2) & (m/z) (+3) ratios

| Composition | mass | m/z (+2) | m/z (+3) |
|---|---|---|---|
| 3200 | 910.327 | 456.1708 | 304.449633 |
| 3210 | 1056.386 | 529.2003 | 353.135967 |
| 3300 | 1113.407 | 557.7108 | 372.142967 |
| 3310 | 1259.465 | 630.7398 | 420.828967 |
| 3320 | 1405.523 | 703.7688 | 469.514967 |
| 3400 | 1316.487 | 659.2508 | 439.8363 |
| 3410 | 1462.544 | 732.2793 | 488.521967 |
| 3420 | 1608.602 | 805.3083 | 537.207967 |
| 3500 | 1519.566 | 760.7903 | 507.5293 |
| 3510 | 1665.624 | 833.8193 | 556.2153 |
| 3520 | 1811.682 | 906.8483 | 604.9013 |
| 3600 | 1722.645 | 862.3298 | 575.2223 |
| 3610 | 1868.703 | 935.3588 | 623.9083 |
| 3620 | 2014.761 | 1008.3878 | 672.5943 |
| 3630 | 2160.89 | 1081.4523 | 721.303967 |
| 3700 | 1925.724642 | 963.869621 | 642.915514 |
| 3710 | 2071.782551 | 1036.898576 | 691.601484 |
| 3720 | 2217.84046 | 1109.92753 | 740.287453 |
| 3730 | 2363.898369 | 1182.956485 | 788.973423 |
| 3740 | 2509.956277 | 1255.985439 | 837.659392 |
| 4200 | 1072.380603 | 537.1976015 | 358.467501 |
| 4210 | 1218.438512 | 610.226556 | 407.153471 |
| 4300 | 1275.459976 | 638.737288 | 426.160625 |
| 4301 | 1566.555392 | 784.284996 | 523.192431 |
| 4310 | 1421.517884 | 711.766242 | 474.846595 |
| 4311 | 1712.613301 | 857.3139505 | 571.8784 |
| 4320 | 1567.575793 | 784.7951965 | 523.532564 |
| 4400 | 1478.539348 | 740.276974 | 493.853749 |
| 4401 | 1769.634765 | 885.8246825 | 590.885555 |
| 4410 | 1624.597257 | 813.3059285 | 542.539719 |
| 4411 | 1915.692673 | 958.8536365 | 639.571524 |
| 4420 | 1770.655166 | 886.334883 | 591.225689 |
| 4421 | 2061.750582 | 1031.882591 | 688.257494 |
| 4430 | 1916.713074 | 959.363837 | 639.911658 |
| 4431 | 2207.808491 | 1104.911546 | 736.943464 |
| 4500 | 1681.618721 | 841.8166605 | 561.546874 |
| 4501 | | 1.0073 | 1.0073 |
| 4510 | 1972.714137 | 987.3643685 | 658.578679 |
| 4511 | 2118.772046 | 1060.393323 | 707.264649 |
| 4520 | 1973.734538 | 987.874569 | 658.918813 |
| 4521 | 2264.829955 | 1133.422278 | 755.950618 |
| 4530 | 2119.792447 | 1060.903524 | 707.604782 |
| 4531 | 2410.887864 | 1206.451232 | 804.636588 |
| 4540 | 2265.850356 | 1133.932478 | 756.290752 |
| 4541 | 2556.945772 | 1279.480186 | 853.322557 |
| 4600 | 1884.698093 | 943.3563465 | 629.239998 |
| 4601 | 2175.79351 | 1088.904055 | 726.271803 |
| 4610 | 2030.756002 | 1016.385301 | 677.925967 |
| 4611 | 2321.851418 | 1161.933009 | 774.957773 |
| 4620 | 2176.813911 | 1089.414256 | 726.611937 |
| 4621 | 2467.909327 | 1234.961964 | 823.643742 |
| 4630 | 2322.87182 | 1162.44321 | 775.297907 |
| 4631 | 2613.967236 | 1307.990918 | 872.329712 |
| 4641 | 2760.025145 | 1381.019873 | 921.015682 |
| 4650 | 2614.987637 | 1308.501119 | 872.669846 |
| 4700 | 2087.777466 | 1044.896033 | 696.933122 |
| 4701 | 2378.872882 | 1190.443741 | 793.964927 |
| 4710 | 2233.835374 | 1117.924987 | 745.619091 |
| 4711 | 2524.930791 | 1263.472696 | 842.650897 |
| 4720 | 2379.893283 | 1190.953942 | 794.305061 |
| 4730 | 2525.951192 | 1263.982896 | 842.991031 |
| 5200 | 1234.433426 | 618.224013 | 412.485109 |
| 5210 | 1380.491335 | 691.2529675 | 461.171078 |
| 5300 | 1437.512799 | 719.7636995 | 480.178233 |
| 5301 | 1728.608215 | 865.3114075 | 577.210038 |
| 5310 | 1583.570708 | 792.792654 | 528.864203 |
| 5311 | 1874.666124 | 938.340362 | 625.896008 |
| 5320 | 1729.628617 | 865.8216085 | 577.550172 |
| 5400 | 1640.592171 | 821.3033855 | 547.871357 |
| 5401 | 1931.687588 | 966.851094 | 644.903163 |
| 5402 | 2222.783005 | 1112.398803 | 741.934968 |
| 5410 | 1786.65008 | 894.33234 | 596.557327 |
| 5411 | 2077.745497 | 1039.880049 | 693.589132 |
| 5412 | 2368.840913 | 1185.427757 | 790.620938 |
| 5420 | 1932.707989 | 967.3612945 | 645.243296 |
| 5421 | 2223.803406 | 1112.909003 | 742.275102 |
| 5430 | 2078.765898 | 1040.390249 | 693.929266 |
| 5431 | 2369.861314 | 1185.937957 | 790.961071 |
| 5432 | 2660.956731 | 1331.485666 | 887.992877 |
| 5500 | 1843.671544 | 922.843072 | 615.564481 |
| 5501 | 2134.766961 | 1068.390781 | 712.596287 |
| 5502 | 2425.862377 | 1213.938489 | 809.628092 |
| 5510 | 1989.729453 | 995.8720265 | 664.250451 |
| 5511 | 2280.824869 | 1141.419735 | 761.282256 |
| 5512 | 2571.920286 | 1286.967443 | 858.314062 |
| 5520 | 2135.787362 | 1068.900981 | 712.936421 |
| 5521 | 2426.882778 | 1214.448689 | 809.968226 |
| 5522 | 2717.978195 | 1359.996398 | 907.000032 |
| 5530 | 2281.84527 | 1141.929935 | 761.62239 |
| 5531 | 2572.940687 | 1287.477644 | 858.654196 |
| 5541 | 2718.998596 | 1360.506598 | 907.340165 |
| 5600 | 2046.750917 | 1024.382759 | 683.257606 |
| 5601 | 2337.846333 | 1169.930467 | 780.289411 |
| 5602 | 2628.94175 | 1315.478175 | 877.321217 |
| 5610 | 2192.808825 | 1097.411713 | 731.943575 |
| 5611 | 2483.904242 | 1242.959421 | 828.975381 |
| 5612 | 2774.999658 | 1388.507129 | 926.007186 |
| 5620 | 2338.866734 | 1170.440667 | 780.629545 |
| 5621 | 2629.962151 | 1315.988376 | 877.66135 |
| 5631 | 2776.020059 | 1389.01733 | 926.34732 |
| 5650 | 2777.040461 | 1389.527531 | 926.687454 |
| 5700 | 2249.830289 | 1125.922445 | 750.95073 |
| 5701 | 2540.925706 | 1271.470153 | 847.982535 |
| 5702 | 2832.021122 | 1417.017861 | 945.014341 |
| 5710 | 2395.888198 | 1198.951399 | 799.636699 |
| 5711 | 2686.983614 | 1344.499107 | 896.668505 |
| 5712 | 2978.079031 | 1490.046816 | 993.70031 |
| 5720 | 2541.946107 | 1271.980354 | 848.322669 |
| 5721 | 2833.041523 | 1417.528062 | 945.354474 |
| 5730 | 2688.004016 | 1345.009308 | 897.008639 |
| 5731 | 2979.099432 | 1490.557016 | 994.040444 |
| 6200 | 1396.48625 | 699.250425 | 466.502717 |
| 6210 | 1542.544159 | 772.2793795 | 515.188686 |
| 6300 | 1599.565622 | 800.790111 | 534.195841 |
| 6301 | 1890.661039 | 946.3378195 | 631.227646 |
| 6310 | 1745.623531 | 873.8190655 | 582.88181 |
| 6311 | 2036.718948 | 1019.366774 | 679.913616 |
| 6320 | 1891.68144 | 946.84802 | 631.56778 |
| 6400 | 1802.644995 | 902.3297975 | 601.888965 |
| 6401 | 2093.740411 | 1047.877506 | 698.92077 |
| 6402 | 2384.835828 | 1193.425214 | 795.952576 |
| 6410 | 1948.702904 | 975.358752 | 650.574935 |
| 6411 | 2239.79832 | 1120.90646 | 747.60674 |
| 6412 | 2530.893737 | 1266.454169 | 844.638546 |
| 6420 | 2094.760813 | 1048.387707 | 699.260904 |
| 6421 | 2385.856229 | 1193.935415 | 796.29271 |
| 6432 | 2823.009554 | 1412.512077 | 942.010485 |
| 6500 | 2005.724367 | 1003.869484 | 669.582089 |
| 6501 | 2296.819784 | 1149.417192 | 766.613895 |
| 6502 | 2587.9152 | 1294.9649 | 863.6457 |
| 6503 | 2879.010617 | 1440.512609 | 960.677506 |
| 6510 | 2151.782276 | 1076.898438 | 718.268059 |
| 6511 | 2442.877693 | 1222.446147 | 815.299864 |
| 6512 | 2733.973109 | 1367.993855 | 912.33167 |
| 6513 | 3025.068526 | 1513.541563 | 1009.36348 |
| 6520 | 2297.840185 | 1149.927393 | 766.954028 |
| 6521 | 2588.935602 | 1295.475101 | 863.985834 |
| 6522 | 2880.031018 | 1441.022809 | 961.017639 |
| 6530 | 2443.898094 | 1222.956347 | 815.639998 |
| 6531 | 2734.99351 | 1368.504055 | 912.671803 |
| 6532 | 3026.088927 | 1514.051764 | 1009.70361 |
| 6540 | 2589.956003 | 1295.985302 | 864.325968 |
| 6541 | 2881.051419 | 1441.53301 | 961.357773 |
| 6600 | 2208.80374 | 1105.40917 | 737.275213 |
| 6601 | 2499.899157 | 1250.956879 | 834.307019 |
| 6602 | 2790.994573 | 1396.504587 | 931.338824 |
| 6603 | 3082.08999 | 1542.052295 | 1028.37063 |
| 6610 | 2354.861649 | 1178.438125 | 785.961183 |
| 6611 | 2645.957065 | 1323.985833 | 882.992988 |
| 6612 | 2937.052482 | 1469.533541 | 980.024794 |
| 6613 | 3228.147898 | 1615.081249 | 1077.0566 |
| 6620 | 2500.919558 | 1251.467079 | 834.647153 |
| 6621 | 2792.014974 | 1397.014787 | 931.678958 |
| 6622 | 3083.110391 | 1542.562496 | 1028.71076 |
| 6623 | 3374.205807 | 1688.110204 | 1125.74257 |
| 6630 | 2646.977466 | 1324.496033 | 883.333122 |
| 6631 | 2938.072883 | 1470.043742 | 980.364928 |
| 6632 | 3229.168299 | 1615.59145 | 1077.39673 |
| 6640 | 2793.035375 | 1397.524988 | 932.019092 |
| 6641 | 3084.130792 | 1543.072696 | 1029.0509 |
| 6642 | 3375.226208 | 1688.620404 | 1126.0827 |
| 6652 | 3521.284117 | 1761.649359 | 1174.76867 |
| 6700 | 2411.883113 | 1206.948857 | 804.968338 |
| 6701 | 2702.978529 | 1352.496565 | 902.000143 |
| 6703 | 3285.169362 | 1643.591981 | 1096.06375 |
| 6710 | 2557.941021 | 1279.977811 | 853.654307 |
| 6711 | 2849.036438 | 1425.525519 | 950.686113 |
| 6711 | 2849.036438 | 1425.525519 | 950.686113 |
| 6712 | 3140.131854 | 1571.073227 | 1047.71792 |
| 6713 | 3431.227271 | 1716.620936 | 1144.74972 |
| 6713 | 3431.227271 | 1716.620936 | 1144.74972 |
| 6720 | 2703.99893 | 1353.006765 | 902.340277 |
| 6721 | 2995.094347 | 1498.554474 | 999.372082 |
| 6721 | 2995.094347 | 1498.554474 | 999.372082 |
| 6730 | 2850.056839 | 1426.03572 | 951.026246 |
| 6731 | 3141.152255 | 1571.583428 | 1048.05805 |
| 6740 | 2996.114748 | 1499.064674 | 999.712216 |
| 7200 | 1558.539073 | 780.2768365 | 520.520324 |
| 7210 | 1704.596982 | 853.305791 | 569.206294 |
| 7400 | 1964.697818 | 983.356209 | 655.906573 |
| 7401 | 2255.793235 | 1128.903918 | 752.938378 |
| 7410 | 2110.755727 | 1056.385164 | 704.592542 |
| 7411 | 2401.851144 | 1201.932872 | 801.624348 |
| 7412 | 2692.94656 | 1347.48058 | 898.656153 |
| 7420 | 2256.813636 | 1129.414118 | 753.278512 |
| 7421 | 2547.909052 | 1274.961826 | 850.310317 |
| 7430 | 2402.871545 | 1202.443073 | 801.964482 |
| 7431 | 2693.966961 | 1347.990781 | 898.996287 |
| 7432 | 2985.062378 | 1493.538489 | 996.028093 |
| 7500 | 2167.777191 | 1084.895896 | 723.599697 |
| 7501 | 2458.872607 | 1230.443604 | 820.631502 |
| 7510 | 2313.8351 | 1157.92485 | 772.285667 |
| 7511 | 2604.930516 | 1303.472558 | 869.317472 |
| 7512 | 2896.025933 | 1449.020267 | 966.349278 |
| 7600 | 2370.856563 | 1186.435582 | 791.292821 |
| 7601 | 2661.95198 | 1331.98329 | 888.324627 |
| 7602 | 2953.047396 | 1477.530998 | 985.356432 |
| 7603 | 3244.142813 | 1623.078707 | 1082.38824 |
| 7604 | 3535.23823 | 1768.626415 | 1179.42004 |
| 7610 | 2516.914472 | 1259.464536 | 839.978791 |
| 7611 | 2808.009889 | 1405.012245 | 937.010596 |
| 7612 | 3099.105305 | 1550.559953 | 1034.0424 |
| 7613 | 3390.200722 | 1696.107661 | 1131.07421 |
| 7614 | 3681.296138 | 1841.655369 | 1228.10601 |
| 7620 | 2662.972381 | 1332.493491 | 888.66476 |
| 7621 | 2954.067798 | 1478.041199 | 985.696566 |
| 7622 | 3245.163214 | 1623.588907 | 1082.72837 |
| 7623 | 3536.258631 | 1769.136616 | 1179.76018 |
| 7632 | 3391.221123 | 1696.617862 | 1131.41434 |
| 7640 | 2955.088199 | 1478.5514 | 986.0367 |
| 7700 | 2573.935936 | 1287.975268 | 858.985945 |
| 7701 | 2865.031352 | 1433.522976 | 956.017751 |
| 7702 | 3156.126769 | 1579.070685 | 1053.04956 |
| 7703 | 3447.222186 | 1724.618393 | 1150.08136 |
| 7710 | 2719.993845 | 1361.004223 | 907.671915 |
| 7711 | 3011.089261 | 1506.551931 | 1004.70372 |
| 7712 | 3302.184678 | 1652.099639 | 1101.73553 |
| 7713 | 3593.280094 | 1797.647347 | 1198.76733 |
| 7714 | 3884.375511 | 1943.195056 | 1295.79914 |
| 7720 | 2866.051754 | 1434.033177 | 956.357885 |
| 7721 | 3157.14717 | 1579.580885 | 1053.38969 |
| 7722 | 3448.242587 | 1725.128594 | 1150.4215 |
| 7730 | 3012.109662 | 1507.062131 | 1005.04385 |
| 7731 | 3303.205079 | 1652.60984 | 1102.07566 |
| 7732 | 3594.300495 | 1798.157548 | 1199.10747 |
| 7740 | 3158.167571 | 1580.091086 | 1053.72982 |
| 7741 | 3449.262988 | 1725.638794 | 1150.76163 |
| 7751 | 3595.320897 | 1798.667749 | 1199.4476 |
| 8200 | 1720.591897 | 861.3032485 | 574.537932 |
| 9200 | 1882.64472 | 942.32966 | 628.55554 |
| 9210 | 2028.702629 | 1015.358615 | 677.24151 |
| 10200 | 2044.697544 | 1023.356072 | 682.573148 |
| 11200 | 2206.750367 | 1104.382484 | 736.590756 |
| 12200 | 2368.80319 | 1185.408895 | 790.608363 |

TABLE 5
Glycan Residue Compound Numbers,
Molecular Mass, and Classification

| Compound | Glycan Mass | Glycan Composition | Class |
|---|---|---|---|
| 3200 | 910.328 | GlcNAc₂Man₃ | HM |
| 3200 | | | |
| 3210 | 1056.386 | GlcNAc₂Man₃Fuc₁ | HM-F |
| 3210 | | | |
| 3300 | 1113.407 | Hex₃HexNAc₃ | C |
| 3300 | | | |
| 3310 | 1259.465 | Hex₃HexNAc₃Fuc₁ | C-F |
| 3310 | | | |
| 3320 | 1405.523 | Hex₃HexNAc₃Fuc₂ | C-F |
| 3400 | 1316.487 | Hex₃HexNAc₄ | C |
| 3410 | 1462.544 | Hex₃HexNAc₄Fuc₁ | C-F |
| 3410 | | | |
| 3420 | 1608.602 | Hex₃HexNAc₄Fuc₂ | C-F |
| 3500 | 1519.566 | Hex₃HexNAc₅ | C |
| 3510 | 1665.624 | Hex₃HexNAc₅Fuc₁ | C-F |
| 3520 | 1811.682 | Hex₃HexNAc₅Fuc₂ | C-F |
| 3600 | 1722.645 | Hex₃HexNAc₆ | C |
| 3610 | 1868.703 | Hex₃HexNAc₆Fuc₁ | C-F |
| 3620 | 2014.761 | Hex₃HexNAc₆Fuc₂ | C-F |
| 3630 | 2160.819 | Hex₃HexNAc₆Fuc₃ | C-F |
| 3700 | 1925.725 | Hex₃HexNAc₇ | C |
| 3710 | 2071.783 | Hex₃HexNAc₇Fuc₁ | C-F |
| 3720 | 2217.841 | Hex₃HexNAc₇Fuc₂ | C-F |
| 3720 | 2217.841 | Hex₃HexNAc₇Fuc₂ | C-F |
| 3730 | 2363.898 | Hex₃HexNAc₇Fuc₃ | C-F |
| 3740 | 2509.956 | Hex₃HexNAc₇Fuc₄ | C-F |
| 4200 | 1072.381 | GlcNAc₂Man₄ | HM |
| 4200 | | | |
| 4210 | 1218.438 | GlcNAc₂Man₄Fuc₁ | HM-F |
| 4210 | | | |
| 4300 | 1275.460 | Hex₄HexNAc₃ | C/H |
| 4300 | | | |
| 4301 | 1566.555 | Hex₄HexNAc₃Neu5Ac₁ | C-S |
| 4301 | 1566.555 | Hex₄HexNAc₃Neu5Ac₁ | C-S |
| 4301 | | | |
| 4310 | 1421.518 | Hex₄HexNAc₃Fuc₁ | C/H-F |
| 4310 | 1566.555 | Hex₄HexNAc₃Neu5Ac₁ | C-S |
| 4310 | | | |
| 4311 | 1712.613 | Hex₄HexNAc₃Fuc₁Neu5Ac₁ | C-FS |
| 4311 | | | |
| 4320 | | | |
| 4400 | 1478.539 | Hex₄HexNAc₄ | C/H |
| 4400 | | | |
| 4401 | 1769.635 | Hex₄HexNAc₄Neu5Ac₁ | C-S |
| 4410 | 1624.597 | Hex₄HexNAc₄Fuc₁ | C/H-F |
| 4410 | | | |
| 4411 | 1915.693 | Hex₄HexNAc₄Fuc₁Neu5Ac₁ | C-FS |
| 4411 | | | |
| 4420 | 1770.655 | Hex₄HexNAc₄Fuc₂ | C/H-F |
| 4420 | | | |
| 4421 | 2061.751 | Hex₄HexNAc₄Fuc₂Neu5Ac₁ | C-FS |
| 4430 | 1916.713 | Hex₄HexNAc₄Fuc₃ | C/H-F |
| 4431 | 2207.808 | Hex₄HexNAc₄Fuc₃Neu5Ac₁ | C-FS |
| 4431 | 2207.808 | Hex₄HexNAc₄Fuc₃Neu5Ac₁ | C-FS |
| 4531 | 2410.888 | Hex₄HexNAc₅Fuc₃Neu5Ac₁ | C-FS |
| 4541 | 2556.946 | Hex₄HexNAc₅Fuc₄Neu5Ac₁ | C-FS |
| 4600 | 1884.698 | Hex₄HexNAc₆ | C |
| 4601 | 2175.794 | Hex₄HexNAc₆Neu5Ac₁ | C-S |
| 4610 | 2030.756 | Hex₄HexNAc₆Fuc₁ | C-F |
| 4611 | 2321.851 | Hex₄HexNAc₆Fuc₁Neu5Ac₁ | C-FS |
| 4620 | 2176.814 | Hex₄HexNAc₆Fuc₂ | C-F |
| 4621 | 2467.909 | Hex₄HexNAc₆Fuc₂Neu5Ac₁ | C-FS |
| 4630 | 2322.872 | Hex₄HexNAc₆Fuc₃ | C-F |
| 4641 | 2760.025 | Hex₄HexNAc₆Fuc₄Neu5Ac₁ | C-FS |
| 4650 | 2614.988 | Hex₄HexNAc₆Fuc₅ | C-F |
| 4700 | 2087.778 | Hex₄HexNAc₇ | C |
| 4701 | 2378.873 | Hex₄HexNAc₇Neu5Ac₁ | C-S |
| 4710 | 2233.835 | Hex₄HexNAc₇Fuc₁ | C-F |
| 4711 | 2524.931 | Hex₄HexNAc₇Fuc₁Neu5Ac₁ | C-FS |
| 4720 | 2379.893 | Hex₄HexNAc₇Fuc₂ | C-F |
| 4730 | 2525.951 | Hex₄HexNAc₇Fuc₃ | C-F |
| 5200 | | | |
| 5200 | | | |
| 5210 | 1380.491 | GlcNAc₂Man₅Fuc₁ | HM-F |
| 5300 | 1437.513 | Hex₅HexNAc₃ | H |
| 5300 | | | |
| 5301 | 1728.608 | Hex₅HexNAc₃Neu5Ac₁ | H-S |
| 5301 | | | |
| 5310 | 1583.571 | Hex₅HexNAc₃Fuc₁ | H-F |
| 5310 | | | |
| 5311 | 1874.666 | Hex₅HexNAc₃Fuc₁Neu5Ac₁ | H-FS |
| 5311 | | | |
| 5320 | 1729.629 | Hex₅HexNAc₃Fuc₂ | H-F |
| 5320 | | | |
| 5400 | | | |
| 5401 | | | |
| 5401 | | | |
| 5402 | | | |
| 5410 | | | |
| 5411 | | Hex₅HexNAc₄Fuc₁Neu5Ac₁ | C-FS |
| 5411 | | | |
| 5412 | | | |
| 5420 | | | |
| 5421 | | | |
| 5430 | | | |
| 5431 | 2369.861 | Hex₅HexNAc₄Fuc₃Neu5Ac₁ | C/H-FS |
| 5432 | 2660.957 | Hex₅HexNAc₄Fuc₃Neu5Ac₂ | C-FS |
| 5432 | 2660.957 | Hex₅HexNAc₄Fuc₃Neu5Ac₂ | C-FS |
| 5531 | 2572.941 | Hex₅HexNAc₅Fuc₃Neu5Ac₁ | C/H-FS |
| 5541 | 2718.999 | Hex₅HexNAc₅Fuc₄Neu5Ac₁ | C-FS |
| 5631 | 2776.020 | Hex₅HexNAc₆Fuc₃Neu5Ac₁ | C-FS |
| 5650 | 2777.040 | Hex₅HexNAc₆Fuc₅ | C-F |
| 5700 | 2249.830 | Hex₅HexNAc₇ | C |
| 5701 | 2540.926 | Hex₅HexNAc₇Neu5Ac₁ | C-S |
| 5702 | 2832.021 | Hex₅HexNAc₇Neu5Ac₂ | C-S |
| 5710 | 2395.888 | Hex₅HexNAc₇Fuc₁ | C-F |
| 5711 | 2686.984 | Hex₅HexNAc₇Fuc₁Neu5Ac₁ | C-FS |
| 5712 | 2978.079 | Hex₅HexNAc₇Fuc₁Neu5Ac₂ | C-FS |
| 5720 | 2541.946 | Hex₅HexNAc₇Fuc₂ | C-F |
| 5721 | 2833.042 | Hex₅HexNAc₇Fuc₂Neu5Ac₁ | C-FS |
| 5730 | 2688.004 | Hex₅HexNAc₇Fuc₃ | C-F |
| 5730 | 2688.004 | Hex₅HexNAc₇Fuc₃ | C-F |
| 5731 | 2979.099 | Hex₅HexNAc₇Fuc₃Neu5Ac₁ | C-FS |
| 6200 | | | |
| 6200 | | | |
| 6210 | 1542.544 | GlcNA₂Man₆Fuc₁ | HM-F |
| 6300 | 1599.566 | Hex₆HexNAc₃ | H |
| 6300 | | | |
| 6301 | 1890.661 | Hex₆HexNAc₃Neu5Ac₁ | H-S |
| 6301 | | | |
| 6310 | 1745.623 | Hex₆HexNAc₃Fuc₁ | H-F |
| 6310 | | | |
| 6311 | 2036.719 | Hex₆HexNAc₃Fuc₁Neu5Ac₁ | H-FS |
| 6311 | 2036.719 | Hex₆HexNAc₃Fuc₁Neu5Ac₁ | H-FS |
| 6311 | | | |
| 6320 | 1891.681 | Hex₆HexNAc₃Fuc₂ | H-F |
| 6400 | 1802.645 | Hex₆HexNAc₄ | H |
| 6401 | 2093.740 | Hex₆HexNAc₄Neu5Ac₁ | H-S |
| 6401 | | | |
| 6402 | 2384.836 | Hex₆HexNAc₄Neu5Ac₂ | H-S |
| 6410 | 1948.703 | Hex₆HexNAc₄Fuc₁ | H-F |
| 6410 | | | |
| 6411 | 2239.798 | Hex₆HexNAc₄Fuc₁Neu5Ac₁ | H-FS |
| 6421 | 2385.856 | Hex₆HexNAc₄Fuc₂Neu5Ac₁ | H-FS |
| 6432 | 2823.009 | Hex₆HexNAc₄Fuc₃Neu5Ac₂ | H-FS |
| 6500 | 2005.724 | Hex₆HexNAc₅ | C/H |
| 6500 | | | |
| 6501 | 2296.820 | Hex₆HexNAc₅Neu5Ac₁ | C/H-S |
| 6501 | | | |
| 6502 | 2587.915 | Hex₆HexNAc₅Neu5Ac₂ | C/H-S |
| 6503 | 2879.011 | Hex₆HexNAc₅Neu5Ac₃ | C-S |
| 6510 | 2151.782 | Hex₆HexNAc₅Fuc₁ | C/H-F |
| 6510 | | | |
| 6511 | 2442.878 | Hex₆HexNAc₅Fuc₁Neu5Ac₁ | C/H-FS |
| 6512 | 2733.973 | Hex₆HexNAc₅Fuc₁Neu5Ac₂ | C/H-FS |
| 6513 | 3025.068 | Hex₆HexNAc₅Fuc₁Neu5Ac₃ | C-FS |
| 6520 | | | |
| 6521 | 2588.936 | Hex₆HexNAc₅Fuc₂Neu5Ac₁ | C/H-FS |
| 6522 | 2880.031 | Hex₆HexNAc₅Fuc₂Neu5Ac₂ | C/H-FS |
| 6530 | 2443.898 | Hex₆HexNAc₅Fuc₃ | C/H-F |
| 6530 | 2879.011 | Hex₆HexNAc₅Neu5Ac₃ | C-S |
| 6531 | 2734.993 | Hex₆HexNAc₅Fuc₃Neu5Ac₁ | C/H-FS |
| 6532 | 3026.089 | Hex₆HexNAc₅Fuc₃Neu5Ac₂ | C/H-FS |
| 6603 | 3082.090 | Hex₆HexNAc₆Neu5Ac₃ | C-S |
| 6623 | 3374.206 | Hex₆HexNAc₆Fuc₂Neu5Ac₃ | C-FS |
| 6630 | 3082.090 | Hex₆HexNAc₆Neu5Ac₃ | C-S |
| 6631 | 2938.073 | Hex₆HexNAc₆Fuc₃Neu5Ac₁ | C-FS |
| 6632 | 3229.168 | Hex₆HexNAc₆Fuc₃Neu5Ac₂ | C-FS |
| 6641 | 3084.131 | Hex₆HexNAc₆Fuc₄Neu5Ac₁ | C-FS |
| 6642 | 3375.226 | Hex₆HexNAc₆Fuc₄Neu5Ac₂ | C-FS |
| 6652 | 3521.284 | Hex₆HexNAc₆Fuc₅Neu5Ac₂ | C-FS |
| 6713 | 3431.227 | Hex₆HexNAc₇Fuc₁Neu5Ac₃ | C-FS |
| 6731 | 3141.152 | Hex₆HexNAc₇Fuc₃Neu5Ac₁ | C-FS |
| 6740 | 2996.115 | Hex₆HexNAc₇Fuc₄ | C-F |
| 7200 | 1558.539 | GlcNAc₂Man₇ | HM |
| 7200 | | | |
| 7200 | | | |
| 7210 | 1704.597 | GlcNAc₂Man₇Fuc₁ | HM-F |
| 7400 | 1964.698 | Hex₇HexNAc₄ | H |
| 7400 | | | |
| 7401 | 2255.793 | Hex₇HexNAc₄Neu5Ac₁ | H-S |
| 7410 | 2110.756 | Hex₇HexNAc₄Fuc₁ | H-F |
| 7411 | 2401.851 | Hex₇HexNAc₄Fuc₁Neu5Ac₁ | H-FS |
| 7412 | 2692.946 | Hex₇HexNAc₄Fuc₁Neu5Ac₂ | H-FS |
| 7420 | 2256.814 | Hex₇HexNAc₄Fuc₂ | H-F |
| 7421 | 2547.909 | Hex₇HexNAc₄Fuc₂Neu5Ac₁ | H-FS |
| 7430 | 2402.871 | Hex₇HexNAc₄Fuc₃ | H-F |
| 7431 | 2693.967 | Hex₇HexNAc₄Fuc₃Neu5Ac₁ | H-FS |
| 7432 | 2985.062 | Hex₇HexNAc₄Fuc₃Neu5Ac₂ | H-FS |
| 7500 | 2167.777 | Hex₇HexNAc₅ | H |
| 7500 | 2167.777 | Hex₇HexNAc₅ | H |
| 7511 | 2604.930 | Hex₇HexNAc₅Fuc₁Neu5Ac₁ | H-FS |
| 7512 | 2896.026 | Hex₇HexNAc₅Fuc₁Neu5Ac₂ | H-FS |
| 7601 | 2661.952 | Hex₇HexNAc₆Neu5Ac₁ | C-S |
| 7602 | 2953.047 | Hex₇HexNAc₆Neu5Ac₂ | C-S |
| 7610 | 2516.914 | Hex₇HexNAc₆Fuc₁ | C-F |
| 7610 | | | |
| 7611 | 2808.010 | Hex₇HexNAc₆Fuc₁Neu5Ac₁ | C-FS |
| 7611 | | | |
| 7612 | 3099.105 | Hex₇HexNAc₆Fuc₁Neu5Ac₂ | C-FS |
| 7613 | 3390.201 | Hex₇HexNAc₆Fuc₁Neu5Ac₃ | C-FS |
| 7620 | 2662.972 | Hex₇HexNAc₆Fuc₂ | C-F |
| 7621 | 2954.068 | Hex₇HexNAc₆Fuc₂Neu5Ac₁ | C-FS |
| 7640 | 2955.088 | Hex₇HexNAc₆Fuc₄ | C-F |
| 7713 | 3593.280 | Hex₇HexNAc₇Fuc₁Neu5Ac₃ | C-FS |
| 7731 | 3303.205 | Hex₇HexNAc₇Fuc₃Neu5Ac₁ | C-FS |
| 7740 | 3158.168 | Hex₇HexNAc₇Fuc₄ | C-F |
| 7741 | 3449.263 | Hex₇HexNAc₇Fuc₄Neu5Ac₁ | C-FS |
| 8200 | 1720.592 | GlcNAc₂Man₈ | HM |
| 8200 | | GlcNAc₂Man₈ | |
| 8200 | | | |
| 9200 | 1882.645 | GlcNAc₂Man₉ | HM |
| 9200 | | GlcNAc₂Man₉ | |
| 9200 | | | |
| 9210 | 2028.702 | GlcNAc₂Man₉Fuc₁ | HM-F |
| 9210 | 2028.702 | GlcNAc₂Man₉Fuc₁ | HM-F |
| 10200 | 2044.697 | GlcNAc₂Man₁₀ | HM |
| 10200 | | | |
| 11200 | | | |

TABLE 1B
Composition of samples

| | Healthy controls | Benign ovarian tumor | EOC |
|---|---|---|---|
| N | 55 | 151 | 145 |
| EOC Stage 1 | | | 12 |
| EOC Stage 2 | | | 6 |
| EOC Stage 3 | | | 68 |
| EOC Stage 4 | | | 12 |
| undocumented | | | 47 |
| Age (median) | 52 | 60 | 66 |

TABLE 2B
Table of IPA-derived Enriched Canonical Pathways. List of
19 enriched canonical pathways found in common among
all study contrasts-benign disease vs. healthy, early disease
vs. healthy and late disease vs. healthy. Scores represent the mean
enrichment score (−log(p-value) across all contrasts.

| Canonical Pathway | Score |
|---|---|
| LXR/RXR Activation | 27.10 |
| FXR/RXR Activation | 27.00 |
| Acute Phase Response Signaling | 23.97 |
| Complement System | 10.11 |
| Atherosclerosis Signaling | 10.43 |
| Clathrin-mediated Endocytosis Signaling | 10.37 |
| IL-12 Signaling and Production in Macrophages | 10.22 |
| Production of Nitric Oxide and Reactive Oxygen Species in | 8.99 |
| Maturity Onset Diabetes of Young (MODY) Signaling | 7.47 |
| Primary Immunodeficiency Signaling | 3.91 |
| Coagulation System | 6.85 |
| Iron homeostasis signaling pathway | 3.85 |
| Systemic Lupus Erythematosus Signaling | 3.20 |
| Neuroprotective Role of THOP1 in Alzheimer's Disease | 2.45 |
| Airway Pathology in Chronic Obstructive | 2.83 |
| Pulmonary Disease | |
| Phagosome Formation | 2.02 |
| Hepatic Fibrosis/Hepatic Stellate Cell Activation | 1.87 |
| TR/RXR Activation | 1.92 |
| Role of Macrophages, Fibroblasts and | 1.61 |
| Endothelial Cells in Rheumatoid | |

Table 6. Sequences

Peptide sequences are recited herein in Table 6. Peptide sequences are described using common 1 letter abbreviations.

| SEQ | | |
|---|---|---|
| ID | | |
| NO. | Compound Name | Peptide Sequence |
| 1 | A1AT-GP001_107_6513 | ADTHDEILEGLNFNLTEIPEAQ |
| | | IHEGFQELLR |
| 2 | A2MG-GP004_1424_5411 | VSNQTLSLFFTVLQDVPVR |
| 3 | A2MG-GP004_55_5411 | GCVLLSYLNETVTVSASLESVR |
| 4 | AACT-GP005_106_7614 | FNLTETSEAEIHQSFQHLLR |
| 5 | AACT-GP005_271_6513 | YTGNASALFILPDQDK |
| 6 | AGP1-GP007_103_7603 | ENGTISR |
| 7 | AGP1-GP007_103_8704 | ENGTISR |
| 8 | AGP1-GP007_103_9804 | ENGTISR |
| 9 | AGP1-GP007_93_7614 | QDQCIYNTTYLNVQR |
| 10 | APOD-GP014_98_5411 | ADGTVNQIEGEATPVNLTEPAK |
| 11 | APOD-GP014_98_9800 | ADGTVNQIEGEATPVNLTEPAK |
| 12 | C4BPA-GP076_221_5402 | FSLLGHASISCTVENETIGVWR |
| | | PSPPTCEK |
| 13 | CERU-GP023_138_6521 | EHEGAIYPDNTTDFQR |
| 14 | CO2_621_5200 | QSVPAHFVALNGSK |
| 15 | FETUA-GP036_176_5401 | AALAAFNAQNNGSNFQLEEISR |
| 16 | FETUA-GP036_176_6513 | AALAAFNAQNNGSNFQLEEISR |
| 17 | FETUA-GP036_346_1102 | TVVQPSVGAAAGPVVPPCPGR |
| 18 | HEMO-GP042_453_5402/ | ALPQPQNVTSLLGCTH |
| | 5421 | |
| 19 | IgG1-GP048_297_3410 | EEQYNSTYR |
| 20 | IgG1-GP048_297_5510 | EEQYNSTYR |
| 21 | IgG2-GP049_297_4510 | EEQFNSTFR |
| 22 | IgG2-GP049_297_5400 | EEQFNSTFR |
| 23 | IgG2-GP049_297_5510 | EEQFNSTFR |
| 24 | PON1-GP060_324_6501 | VTQVYAENGTVLQGSTVASVYK |
| 25 | QuantPep-A2GL-GP003_ | DLLLPQPDLR |
| | DLLLPQPDLR | |
| 26 | QuantPep-AFAM- | SDVGFLPPFPTLDPEEK |
| | GP006_SDVGFLPPFPTLDPEEK | |
| 27 | QuantPep-CAN3-GP022_ | FIIDGANR |
| | FIIDGANR | |
| 28 | QuantPep-TTR- | TSESGELHGLTTEEEFVEGIYK |
| | GP065_ | |
| | TSESGELHGLTTEEEFVEGIYK | |
| 29 | QuantPep-UN13A- | LDLGLTVEVWNK |
| | GP066_LDLGLTVEVWNK | |
| 30 | TRFE-GP064_432_6501 | CGLVPVLAENYNK |
| 31 | TRFE-GP064_432_6502 | CGLVPVLAENYNK |
| 32 | TRFE-GP064_432_6503 | CGLVPVLAENYNK |
| 33 | TRFE-GP064_630_5400 | QQQHLFGSNVTDCSGNFCLFR |
| 34 | TRFE-GP064_630_5411 | QQQHLFGSNVTDCSGNFCLFR |
| 35 | TRFE-GP064_630_6502 | QQQHLFGSNVTDCSGNFCLFR |
| 36 | TRFE-GP064_630_6513 | QQQHLFGSNVTDCSGNFCLFR |
| 37 | VTNC-GP067_169_5401 | NGSLFAFR |
| 38 | ZA2G-GP068_128_5402 | FGCEIENNR |

Table 1C provide alternative names of the biomarkers described here. Both Name 1 and Name 2 are alternatively used to describe the same biomarker.

TABLE 1C
Biomarkers

| Name 1 | Name 2 |
|---|---|
| A1AT_107_6513 | A1AT.GP001_107_6513 |
| A2MG_1424_5411 | A2MG.GP004_1424_5411 |
| A2MG_55_5411 | A2MG.GP004_55_5411 |
| AACT_106_7614 | AACT.GP005_106_7614 |
| AACT_271_6513 | AACT.GP005_271_6513 |
| AGP1_103_7603 | AGP1.GP007_103_7603 |
| AGP1_103_8704 | AGP1.GP007_103_8704 |
| AGP1_103_9804 | AGP1.GP007_103_9804 |
| AGP1_93_7614 | AGP1.GP007_93_7614 |
| APOD_98_5411 | APOD.GP014_98_5411 |
| C4BPA_221_5402 | C4BPA.GP076_221_5402 |
| CERU_138_6502 | CERU.GP023_138_6521 |
| CO2_621_5200 | CO2_621_5200 |
| FETUA_176_5401 | FETUA.GP036_176_5401 |
| FETUA_176_6513 | FETUA.GP036_176_6513 |
| FETUA_346_1102 | FETUA.GP036_346_1102 |
| HEMO_453_5402 | HEMO.GP042_453_5402.5421 |
| IGG1_297_3410 | IGG1.GP048_297_3410 |
| IGG1_297_5510 | IGG1.GP048_297_5510 |
| IGG2_297_4510 | IGG2.GP049_297_4510 |
| IGG2_297_5400 | IGG2.GP049_297_5400 |
| IGG2_297_5510 | IGG2.GP049_297_5510 |
| QUANTPEP.A2GL_ | QUANTPEP.A2GL.GP003_ |
| DLLLPQPDLR | DLLLPQPDLR |
| QUANTPEP.AFAM_ | QUANTPEP.AFAM.GP006_ |
| SDVGFLPPFPTLDPEEK | SDVGFLPPFPTLDPEEK |
| QUANTPEP.TTR_ | QUANTPEP.TTR.GP065_ |
| TSESGELHGLT- | TSESGELHGLT- |
| TEEEFVEGIYK | TEEEFVEGIYK |
| TRFE_432_6501 | TRFE.GP064_432_6501 |
| TRFE_432_6502 | TRFE.GP064_432_6502 |
| TRFE_432_6503 | TRFE.GP064_432_6503 |
| TRFE_630_5400 | TRFE.GP064_630_5400 |
| TRFE_630_5411 | TRFE.GP064_630_5411 |
| TRFE_630_6502 | TRFE.GP064_630_6502 |
| TRFE_630_6513 | TRFE.GP064_630_6513 |
| VTNC_169_5401 | VTNC.GP067_169_5401 |
| ZA2G_128_5402 | ZA2G.GP068_128_5402 |
| HPT_241_5402 | APOD-GP014_98_9800 |
| HPT_184_5402 | PON1-GP060_324_6501 |
| PEP-APOA1_ | QuantPep-CAN3- |
| THLAPYSDELR | GP022_FIIDGANR |
| PEP-APOA1_ | QuantPep-UN13A-GP066_ |
| VSFLSALEEYTK | LDLGLTVEVWNK |

What is claimed is:

1. A method for diagnosing a subject with respect to an ovarian cancer disease state, the method comprising
receiving peptide structure data corresponding to a biological sample obtained from the subject;
analyzing the peptide structure data using a supervised machine learning model to generate a disease indicator that indicates whether the biological sample evidences an ovarian cancer disease state based on at least three peptide structures selected from one of a first group of peptide structures identified in Table 1A and a second group of peptide structures identified in Table 2A,
wherein the first group of peptide structures and the second group of peptide structures are associated with the ovarian cancer disease state;
wherein each of the first group of peptide structures in Table 1A and the second group of peptide structures in Table 2A is listed in order of relative significance to the disease indicator; and
generating a diagnosis output based on the disease indicator.

2. The method of claim 1, wherein the disease indicator comprises a score.

3. The method of claim 2, wherein generating the diagnosis output comprises
determining that the score falls above a selected threshold; and
generating the diagnosis output based on the score falling above the selected threshold, wherein the diagnosis output includes a positive or negative diagnosis for the ovarian cancer disease state.

4. The method of claim 3, wherein the score comprises a probability score and the selected threshold is 0.5.

5. The method of claim 3 or claim 4, wherein the selected threshold falls within a range between 0.30 and 0.65.

6. The method of any one of claims 1-5, wherein analyzing the peptide structure data comprises analyzing the peptide structure data using a binary classification model.

7. The method of any one of claims 1-6, wherein a peptide structure of the at least three peptide structures comprises a glycopeptide structure defined by a peptide sequence and a glycan structure linked to the peptide sequence at a linking site of the peptide sequence, as identified in Table 1A or Table 2A, with the peptide sequence being one of SEQ ID NOS: 111-119 in Table 1A as defined in Table 5A or one of SEQ ID NOS: 114, 115, and 131-146 in Table 2A as defined in Table 5A.

8. The method of any one of claims 1-7, further comprising:
training the supervised machine learning model using training data,
wherein the training data comprises a plurality of peptide structure profiles for a plurality of subjects and a plurality of subject diagnoses for the plurality of subjects.

9. The method of claim 8, wherein the plurality of subject diagnoses includes a positive diagnosis for any subject of the plurality of subjects determined to have the ovarian cancer disease state and a negative diagnosis for any subject of the plurality of subjects determined to have a healthy state or a benign tumor state.

10. The method of any one of claims 8-9, wherein each peptide structure profile of the plurality of peptide structure profiles comprises a feature selected from one the group consisting of a relative abundance and a concentration for a corresponding peptide structure.

11. The method of any one of claims 1-10, wherein the supervised machine learning model comprises a logistic regression model.

12. The method of any one of claims 1-11, wherein the first group of peptide structures in Table 1A is used to distinguish between the ovarian cancer disease state and a healthy state and wherein the second group of peptide structures in Table 2A is used to distinguish between the ovarian cancer disease state and a benign tumor state.

13. The method of any one of claims 1-12, wherein the peptide structure data comprises at least one of an abundance, a relative abundance, a normalized abundance, a relative quantity, an adjusted quantity, a normalized quantity, a relative concentration, an adjusted concentration, or a normalized concentration.

14. A method of training a model to diagnose a subject with respect to an ovarian cancer disease state, the method comprising:
receiving quantification data for a panel of peptide structures for a plurality of biological samples for a plurality of subjects,
wherein the plurality of subjects includes a first portion diagnosed with a negative diagnosis of an ovarian cancer disease state and a second portion diagnosed with a positive diagnosis of the ovarian cancer disease state;
wherein the quantification data comprises a plurality of peptide structure profiles for the plurality of subjects; and
training a machine learning model using the quantification data to diagnose a biological sample with respect to the ovarian cancer disease state using a first group of peptide structures associated with the ovarian cancer disease state or a second group of peptide structures associated with the ovarian cancer disease state,
wherein the first group of peptide structures is identified in Table 1A and listed in Table 1A with respect to relative significance to diagnosing the biological sample; and
wherein the second group of peptide structures is identified in Table 2A and listed in Table 2A with respect to relative significance to diagnosing the biological sample.

15. The method of claim 14, wherein the machine learning model comprises a logistic regression model.

16. The method of any one of claims 14-15, further comprising:
identifying an initial plurality of peptide structure profiles;
filtering the initial plurality of peptide structure profiles by a coefficient of variation to generate a plurality of peptide structure profiles for use in training the machine learning model.

17. The method of claim 16, wherein the filtering is performed to exclude peptide structure profiles having the coefficient of variation at or above 20%.

18. The method of claim 14, wherein training the machine learning model comprises reducing the plurality of peptide structure profiles using LASSO regression to identify a final group of peptide structures identified in Table 1A, or Table 2A.

19. The method of any one of claims 14-18, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

20. A method for diagnosing a subject with respect to an ovarian cancer disease state, the method comprising:
receiving peptide structure data corresponding to a biological sample obtained from the subject;
analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 3A; and
generating a diagnosis output based on the disease indicator.

21. The method of claim 20, wherein the wherein the group of peptide structures in Table 3A is listed in order of relative significance to the disease indicator.

22. The method of claim 20 or claim 21, wherein the disease indicator comprises a score.

23. The method of claim 22, wherein generating the diagnosis output comprises:
determining that the score falls above a selected threshold; and
generating the diagnosis output based on the score falling above the selected threshold, wherein the diagnosis output includes a positive diagnosis for the ovarian cancer disease state.

24. The method of claim 22, wherein generating the diagnosis output comprises:
determining that the score falls below a selected threshold; and
generating the diagnosis output based on the score falling below the selected threshold, wherein the diagnosis output includes a negative diagnosis for the ovarian cancer disease state.

25. The method of claim 23 or claim 24, wherein the score comprises a probability score and the selected threshold is 0.5.

26. The method of claim 23 or claim 24, wherein the selected threshold falls within a range between 0.30 and 0.65.

27. The method of any one of claims 20-26, wherein analyzing the peptide structure data comprises:
analyzing the peptide structure data using a binary classification model.

28. The method of any one of claims 20-27, wherein a peptide structure of the at least three peptide structures comprises a glycopeptide structure defined by a peptide sequence and a glycan structure linked to the peptide sequence at a linking site of the peptide sequence, as identified in Table 3A, with the peptide sequence being one of SEQ ID NOS: 111, 114, 115, 131, 132, 133, 134, 137, 138, 140, 142, 144, 145, 146, 153-165.

29. The method of claim 28, wherein the peptide structure comprises an amino acid sequence set forth in SEQ ID NOS: 111, 114, 115, 131, 132, 133, 134, 137, 138, 140, 142, 144, 145, 146, or 153-165.

30. The method of claim 28 or claim 29, wherein the method comprises analyzing the peptide structure using a supervised machine learning model to generate a disease indicator that indicates whether the biological sample evidences the ovarian cancer disease state of having a malignant pelvic tumor based on at least five, at least 10 at least 15, at least 20, at least 25, at least 30, or at least 35 peptide structures selected from one of a group of peptide structures identified in Table 3A.

31. The method of claim 30, wherein the method comprises analyzing the peptide structure using a supervised machine learning model to generate a disease indicator that indicates whether the biological sample evidences the ovarian cancer disease state of having a malignant pelvic tumor based on each of the peptide structures selected from one of a group of peptide structures identified in Table 3A, comprising an amino acid sequence set forth in SEQ ID NOS: 111, 114, 115, 131, 132, 133, 134, 137, 138, 140, 142, 144, 145, 146, or 153-165.

32. The method of any one of claims 20-31, further comprising:
training the supervised machine learning model using training data,
wherein the training data comprises a plurality of peptide structure profiles for a plurality of subjects and a plurality of subject diagnoses for the plurality of subjects.

33. The method of claim 32, wherein the plurality of subject diagnoses includes a positive diagnosis for any subject of the plurality of subjects determined to have the malignant pelvic tumor and a negative diagnosis for any subject of the plurality of subjects determined to have a healthy state.

34. The method of claim 32, wherein the plurality of subject diagnoses includes a positive diagnosis for any subject of the plurality of subjects determined to have the ovarian cancer disease state and a negative diagnosis for any subject of the plurality of subjects determined to have a benign pelvic tumor.

35. The method of any one of claims 32-34, further comprising:
performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state; and
identifying a training group of peptide structures based on the differential expression analysis for use as prognostic markers for the ovarian cancer disease state; and
forming the training data based on the training group of peptide structures identified.

36. The method of claim 35, wherein training the supervised machine learning model comprises reducing the training group of peptide structures to a final group of peptide structures identified in Table 3A.

37. The method of any one of claims 32-36, wherein each peptide structure profile of the plurality of peptide structure profiles includes a feature selected from one of a relative abundance and a concentration for a corresponding peptide structure.

38. The method of any one of claims 32-37, wherein the plurality of peptide structure profiles includes a first peptide structure profile with a relative abundance for a corresponding peptide structure and a second peptide structure profile with a concentration for the corresponding peptide structure.

39. The method of any one of claims 20-38, wherein the supervised machine learning model comprises a logistic regression model.

40. The method of any one of claims 20-39, wherein the first group of peptide structures in Table 3A is used to distinguish between the ovarian cancer disease state having the malignant pelvic tumor and a non-ovarian cancer state having a benign pelvic tumor.

41. The method of any one of claims 20-40, wherein the peptide structure data comprises quantification data selected from the group consisting of an abundance, a relative abundance, a normalized abundance, a relative quantity, an adjusted quantity, a normalized quantity, a relative concentration, an adjusted concentration, or a normalized concentration.

42. A method of treating ovarian cancer in a subject comprising receiving peptide structure data corresponding to a biological sample obtained from the subject;
analyzing the peptide structure data using a supervised machine learning model to generate a disease indicator that indicates whether the biological sample evidences the ovarian cancer disease state of having a malignant pelvic tumor based on at least three peptide structures selected from one of a group of peptide structures identified in Table 1A, Table 2A, and/or Table 3A; and
generating a diagnosis output based on the disease indicator.

43. The method of claim 42, wherein the disease indicator is based on at least three peptide structures from one of a group of peptide structures identified in Table 3A.

44. The method of any one of claims 42-43, further providing a treatment recommendation based upon the diagnosis.

45. The method of any one of claims 42-44, further comprising administering a treatment for ovarian cancer.

46. The method of any one of claims 1-45, wherein the peptide structure data is generated using multiple reaction monitoring mass spectrometry (MRM-MS).

47. The method of any one of claims 1-46, further comprising:
preparing a sample of the biological sample using reduction, alkylation, and enzymatic digestion to form a prepared sample that includes a set of peptide structures.

48. The method of claim 47, further comprising:
generating the peptide structure data from the prepared sample using multiple reaction monitoring mass spectrometry (MRM-MS).

49. The method of any one of claims 1-13 and 20-48, wherein generating the diagnosis output comprises:
generating a report identifying that the biological sample evidences the ovarian cancer disease state.

50. The method of claim 49, wherein the treatment output comprises at least one of an identification of a treatment to treat the subject or a treatment plan.

51. The method of claim 50, further comprising administering the identified treatment or treatment plan to the subject.

52. The method of any one of claims 42-51, wherein the treatment comprises at least one of surgery, radiation therapy, a targeted drug therapy, chemotherapy, immunotherapy, hormone therapy, or neoadjuvant therapy.

53. The method of any one of claims 1-13 and 20-52, further comprising:
performing a biopsy of the subject in response to the diagnosis output indicating a positive diagnosis for the ovarian cancer disease state.

54. The method of any one of claims 1-13 and 20-53, further comprising:
generating a report recommending that a biopsy be performed for the subject in response to the diagnosis output indicating a positive diagnosis for the ovarian cancer disease state.

55. The method of any one of claims 1-13 and 20-54, further comprising:
performing a biopsy of the subject in response to the diagnosis output indicating a positive diagnosis for the ovarian cancer disease state.

56. A method of training a model to diagnose a subject with respect to an ovarian cancer disease state having a malignant pelvic tumor, the method comprising
receiving quantification data for a panel of peptide structures for a plurality of samples for a plurality of subjects,
wherein the plurality of subjects includes a first portion diagnosed with a negative diagnosis of an ovarian cancer disease state and a second portion diagnosed with a positive diagnosis of the ovarian cancer disease state;
wherein the quantification data comprises a plurality of peptide structure profiles for the plurality of subjects; and
training a machine learning model using the quantification data to diagnose a biological sample with respect to the ovarian cancer disease state using a group of peptide structures associated with the ovarian cancer disease state,
wherein the group of peptide structures is identified in Table 3A and listed in Table 3A with respect to relative significance to diagnosing the biological sample.

57. The method of claim 56, wherein the machine learning model comprises a logistic regression model, optionally a LASSO regression model.

58. The method of any one of claims 56-57, further comprising:
identifying an initial plurality of peptide structure profiles;
filtering the initial plurality of peptide structure profiles by a coefficient of variation to generate a plurality of peptide structure profiles for use in training the machine learning model.

59. The method of claim 58, wherein the filtering is performed to exclude peptide structure profiles having the coefficient of variation at or above 20%.

60. The method of claim 57, wherein training the machine learning model comprises reducing the plurality of peptide structure profiles using LASSO regression to identify a final group of peptide structures identified in Table 3A.

61. The method of any one of claims 1-60, wherein a negative diagnosis for the ovarian cancer disease state indicates a non-ovarian cancer state comprising a benign tumor state.

62. The method of any one of claims 56-61, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

63. The method of any one of claims 56-62, wherein the trained model uses a relative abundance for a first portion of the first group of peptide structures and a concentration for a second portion of the second group of peptide structures.

64. The method of any one of claims 56-63 wherein the training comprises:
identifying a first portion of the plurality of biological samples for subjects with benign pelvic tumors and malignant pelvic tumors and a second portion of the plurality of biological samples for subjects with a healthy status; and
generating a training set of peptide structure profiles for 80% of the first portion and a test set of peptide structure profiles for a remaining 20% of the first portion and the second portion.

65. The method of any one of claims 56-64, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

66. The method of any one of claims 56-65, further comprising:
generating, using the trained machine learning model, a disease indicator for diagnosing the biological sample with respect to the ovarian cancer disease state; and
generating a report recommending that a biopsy be performed for the subject in response to the diagnosis indicator indicating a positive diagnosis for the ovarian cancer disease state.

67. The method of any one of claims 56-66, further comprising:
generating, using the trained machine learning model, a disease indicator for diagnosing the biological sample with respect to the ovarian cancer disease state; and
performing a biopsy of the subject in response to the diagnosis indicator indicating a positive diagnosis for the ovarian cancer disease state.

68. The method of any one of claims 56-66, further comprising:
generating, using the trained machine learning model, a disease indicator for diagnosing the biological sample with respect to the ovarian cancer disease state; and
generating a report recommending that a biopsy be performed for the subject in response to the diagnosis indicator indicating a positive diagnosis for the ovarian cancer disease state.

69. The method of any one of claims 1-68, wherein the ovarian cancer disease state comprises a malignant pelvic tumor.

70. The method of any one of claims 1-69, wherein the ovarian cancer disease state is epithelial ovarian cancer, or optionally malignant epithelial ovarian cancer.

71. The method of any one of claims 1-70, wherein the subject is a human.

72. A kit comprising at least one of a glycopeptide standard, a buffer, or a set of peptide sequences to carry out the method of any one of claims 1-40, a peptide sequence of the set of peptide sequences identified by a corresponding one of SEQ ID NOS: 111-119, defined in Table 1A and Table 5A.

73. A composition comprising at least one of peptide structures PS-1-PS-10 and PS-11-PS-34 from Table 1A and Table 2A.

74. A composition comprising a glycopeptide structure selected as one from a group consisting of peptide structures PS-1, PS-5, PS-11, PS-15, PS-20, PS-25, PS-28, PS-29, PS-30, PS-31, PS-32, and PS-35 to PS-61 identified in Table 3A, wherein:
the peptide structure comprises:
an amino acid peptide sequence identified in Table 5A as corresponding to the peptide structure; and
a glycan structure identified in Table 7A as corresponding to the peptide structure in which the glycan structure is linked to a residue of the amino acid peptide sequence at a corresponding position identified in Table 3A; and
wherein the glycan structure has a glycan composition.

75. A kit comprising at least one agent for quantifying at least one peptide structure identified in Table 3A to carry out the method of any one of claims 20-55.

76. A kit comprising at least one of a glycopeptide standard, a buffer, or a set of peptide sequences to carry out the method of any one of claims 20-52, a peptide sequence of the set of peptide sequences identified by a corresponding one of SEQ ID NOS: 111, 114, 115, 131, 132, 133, 134, 137, 138, 140, 142, 144, 145, 146, 153-165 identified in Table 3A.

77. A system comprising:
one or more data processors; and
a non-transitory computer readable storage medium containing instructions which, when executed on the one or more data processors, cause the one or more data processors to perform part or all of any one of claims 1-13 and 20-55.

78. A computer-program product tangibly embodied in a non-transitory machine-readable storage medium, including instructions configured to cause one or more data processors to perform part or all of any one of claims 1-13 and 20-55.


SEQUENCE LISTING (165 sequences)

SEQ ID NO: 1 - protein, 32 aa, Artificial Sequence
  Synthetic Construct
ADTHDEILEGLNFNLTEIPEAQIHEGFQELLR

SEQ ID NO: 2 - protein, 19 aa, Artificial Sequence
  Synthetic Construct
VSNQTLSLFFTVLQDVPVR

SEQ ID NO: 3 - protein, 22 aa, Artificial Sequence
  Synthetic Construct
GCVLLSYLNETVTVSASLESVR

SEQ ID NO: 4 - protein, 20 aa, Artificial Sequence
  Synthetic Construct
FNLTETSEAEIHQSFQHLLR

SEQ ID NO: 5 - protein, 16 aa, Artificial Sequence
  Synthetic Construct
YTGNASALFILPDQDK

SEQ ID NO: 6 - protein, 7 aa, Artificial Sequence
  Synthetic Construct
ENGTISR

SEQ ID NO: 7 - protein, 7 aa, Artificial Sequence
  Synthetic Construct
ENGTISR

SEQ ID NO: 8 - protein, 7 aa, Artificial Sequence
  Synthetic Construct
ENGTISR

SEQ ID NO: 9 - protein, 15 aa, Artificial Sequence
  Synthetic Construct
QDQCIYNTTYLNVQR

SEQ ID NO: 10 - protein, 22 aa, Artificial Sequence
  Synthetic Construct
ADGTVNQIEGEATPVNLTEPAK

SEQ ID NO: 11 - protein, 22 aa, Artificial Sequence
  Synthetic Construct
ADGTVNQIEGEATPVNLTEPAK

SEQ ID NO: 12 - protein, 30 aa, Artificial Sequence
  Synthetic Construct
FSLLGHASISCTVENETIGVWRPSPPTCEK

SEQ ID NO: 13 - protein, 16 aa, Artificial Sequence
  Synthetic Construct
EHEGAIYPDNTTDFQR

SEQ ID NO: 14 - protein, 14 aa, Artificial Sequence
  Synthetic Construct
QSVPAHFVALNGSK

SEQ ID NO: 15 - protein, 22 aa, Artificial Sequence
  Synthetic Construct
AALAAFNAQNNGSNFQLEEISR

SEQ ID NO: 16 - protein, 22 aa, Artificial Sequence
  Synthetic Construct
AALAAFNAQNNGSNFQLEEISR

SEQ ID NO: 17 - protein, 21 aa, Artificial Sequence
  Synthetic Construct
TVVQPSVGAAAGPVVPPCPGR

SEQ ID NO: 18 - protein, 16 aa, Artificial Sequence
  Synthetic Construct
ALPQPQNVTSLLGCTH

SEQ ID NO: 19 - protein, 9 aa, Artificial Sequence
  Synthetic Construct
EEQYNSTYR

SEQ ID NO: 20 - protein, 9 aa, Artificial Sequence
  Synthetic Construct
EEQYNSTYR

SEQ ID NO: 21 - protein, 9 aa, Artificial Sequence
  Synthetic Construct
EEQFNSTFR

SEQ ID NO: 22 - protein, 9 aa, Artificial Sequence
  Synthetic Construct
EEQFNSTFR

SEQ ID NO: 23 - protein, 9 aa, Artificial Sequence
  Synthetic Construct
EEQFNSTFR

SEQ ID NO: 24 - protein, 22 aa, Artificial Sequence
  Synthetic Construct
VTQVYAENGTVLQGSTVASVYK

SEQ ID NO: 25 - protein, 10 aa, Artificial Sequence
  Synthetic Construct
DLLLPQPDLR

SEQ ID NO: 26 - protein, 17 aa, Artificial Sequence
  Synthetic Construct
SDVGFLPPFPTLDPEEK

SEQ ID NO: 27 - protein, 8 aa, Artificial Sequence
  Synthetic Construct
FIIDGANR

SEQ ID NO: 28 - protein, 22 aa, Artificial Sequence
  Synthetic Construct
TSESGELHGLTTEEEFVEGIYK

SEQ ID NO: 29 - protein, 12 aa, Artificial Sequence
  Synthetic Construct
LDLGLTVEVWNK

SEQ ID NO: 30 - protein, 13 aa, Artificial Sequence
  Synthetic Construct
CGLVPVLAENYNK

SEQ ID NO: 31 - protein, 13 aa, Artificial Sequence
  Synthetic Construct
CGLVPVLAENYNK

SEQ ID NO: 32 - protein, 13 aa, Artificial Sequence
  Synthetic Construct
CGLVPVLAENYNK

SEQ ID NO: 33 - protein, 21 aa, Artificial Sequence
  Synthetic Construct
QQQHLFGSNVTDCSGNFCLFR

SEQ ID NO: 34 - protein, 21 aa, Artificial Sequence
  Synthetic Construct
QQQHLFGSNVTDCSGNFCLFR

SEQ ID NO: 35 - protein, 21 aa, Artificial Sequence
  Synthetic Construct
QQQHLFGSNVTDCSGNFCLFR

SEQ ID NO: 36 - protein, 21 aa, Artificial Sequence
  Synthetic Construct
QQQHLFGSNVTDCSGNFCLFR

SEQ ID NO: 37 - protein, 8 aa, Artificial Sequence
  Synthetic Construct
NGSLFAFR

SEQ ID NO: 38 - protein, 9 aa, Artificial Sequence
  Synthetic Construct
FGCEIENNR

SEQ ID NO: 39-100 - skipped (62 entries)

SEQ ID NO: 101 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 102 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 103 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 104 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 105 - protein, 330 aa, Homo sapiens [sequence omitted]

SEQ ID NO: 106 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 107 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 108 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 109 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 110 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 9 aa, Artificial Sequence
  Synthetic Construct
FGCEIENNR

SEQ ID NO: 112 - protein, 19 aa, Artificial Sequence
  Synthetic Construct
VLSNNSDANLELINTWVAK

SEQ ID NO: 113 - protein, 7 aa, Artificial Sequence
  Synthetic Construct
LISNCSK

SEQ ID NO: 114 - protein, 16 aa, Artificial Sequence
  Synthetic Construct
EHEGAIYPDNTTDFQR

SEQ ID NO: 115 - protein, 9 aa, Artificial Sequence
  Synthetic Construct
EEQYNSTYR

SEQ ID NO: 116 - protein, 22 aa, Artificial Sequence
  Synthetic Construct
CSDGWSFDATTLDDNGTMLFFK

SEQ ID NO: 117 - protein, 33 aa, Artificial Sequence
  Synthetic Construct
QVFPGLNYCTSGAYSNASSTDSASYYPLTGDTR

SEQ ID NO: 118 - protein, 13 aa, Artificial Sequence
  Synthetic Construct
NLFLNHSENATAK

SEQ ID NO: 119 - protein, 9 aa, Artificial Sequence
  Synthetic Construct
EEQYNSTFR

SEQ ID NO: 120 - protein, 752 aa, Homo sapiens
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 121 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 122 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 123 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 124 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 125 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 126 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 127 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 128 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 129 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 130 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 131 - protein, 14 aa, Artificial Sequence
  Synthetic Construct
QSVPAHFVALNGSK

SEQ ID NO: 132 - protein, 15 aa, Artificial Sequence
  Synthetic Construct
QDQCIYNTTYLNVQR

SEQ ID NO: 133 - protein, 16 aa, Artificial Sequence
  Synthetic Construct
YTGNASALFILPDQDK

SEQ ID NO: 134 - protein, 19 aa, Artificial Sequence
  Synthetic Construct
VSNQTLSLFFTVLQDVPVR

SEQ ID NO: 135 - protein, 20 aa, Artificial Sequence
  Synthetic Construct
ENLTAPGSDSAVFFEQGTTR

SEQ ID NO: 136 - protein, 15 aa, Artificial Sequence
  Synthetic Construct
FVEGSHNSTVSLTTK

SEQ ID NO: 137 - protein, 20 aa, Artificial Sequence
  Synthetic Construct
FNLTETSEAEIHQSFQHLLR

SEQ ID NO: 138 - protein, 32 aa, Artificial Sequence; identical to SEQ ID NO: 1
  Synthetic Construct

SEQ ID NO: 139 - protein, 27 aa, Artificial Sequence
  Synthetic Construct
NISDGFDGIPDNVDAALALPAHSYSGR

SEQ ID NO: 140 - protein, 9 aa, Artificial Sequence
  Synthetic Construct
EEQFNSTFR

SEQ ID NO: 141 - protein, 18 aa, Artificial Sequence
  Synthetic Construct
IPCSQPPQIEHGTINSSR

SEQ ID NO: 142 - protein, 7 aa, Artificial Sequence
  Synthetic Construct
ENGTISR

SEQ ID NO: 143 - protein, 11 aa, Artificial Sequence
  Synthetic Construct
LGNWSAMPSCK

SEQ ID NO: 144 - protein, 22 aa, Artificial Sequence
  Synthetic Construct
ADGTVNQIEGEATPVNLTEPAK

SEQ ID NO: 145 - protein, 21 aa, Artificial Sequence
  Synthetic Construct
QQQHLFGSNVTDCSGNFCLFR

SEQ ID NO: 146 - protein, 22 aa, Artificial Sequence
  Synthetic Construct
GCVLLSYLNETVTVSASLESVR

SEQ ID NO: 147 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 148 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 149 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 150 - protein, 599 aa, Homo sapiens
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 151 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 152 - protein, 597 aa, Homo sapiens
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 153 - protein, 8 aa, Artificial Sequence
  Synthetic Construct
NGSLFAFR

SEQ ID NO: 154 - protein, 22 aa, Artificial Sequence
  Synthetic Construct
AALAAFNAQNNGSNFQLEEISR

SEQ ID NO: 155 - protein, 10 aa, Artificial Sequence
  Synthetic Construct
DLLLPQPDLR

SEQ ID NO: 156 - protein, 24 aa, Artificial Sequence
  Synthetic Construct
MVSHHNLTTGATLINEQWLLTTAK

SEQ ID NO: 157 - protein, 13 aa, Artificial Sequence
  Synthetic Construct
CGLVPVLAENYNK

SEQ ID NO: 158 - protein, 16 aa, Artificial Sequence
  Synthetic Construct
ALPQPQNVTSLLGCTH

SEQ ID NO: 159 - protein, 22 aa, Artificial Sequence
  Synthetic Construct
TSESGELHGLTTEEEFVEGIYK

SEQ ID NO: 160 - protein, 16 aa, Artificial Sequence
  Synthetic Construct
VVLHPNYSQVDIGLIK

SEQ ID NO: 161 - protein, 17 aa, Artificial Sequence
  Synthetic Construct
SDVGFLPPFPTLDPEEK

SEQ ID NO: 162 - protein, 12 aa, Artificial Sequence
  Synthetic Construct
VSFLSALEEYTK

SEQ ID NO: 163 - protein, 21 aa, Artificial Sequence
  Synthetic Construct
TVVQPSVGAAAGPVVPPCPGR

SEQ ID NO: 164 - protein, 11 aa, Artificial Sequence
  Synthetic Construct
THLAPYSDELR

SEQ ID NO: 165 - protein, 30 aa, Artificial Sequence; identical to SEQ ID NO: 12
  Synthetic Construct